(19) United States
(12) Patent Application Publication
(10) Pub. No.: US 2009/0042854 A1
(43) Pub. Date: Feb. 12, 2009

(54) Hexahydro-3H-Pyrrolizin-3-Ones Useful as Tachykinin Receptor Antagonists

(76) Inventors: Jianming Bao, Scotch Plains, NJ (US); Robert J. DeVita, Westfield, NJ (US); Huagang Lu, Plainsboro, NJ (US); Sander G. Mills, Scotch Plains, NJ (US); Gregori J. Morriello, Randolph, NJ (US)

Correspondence Address: MERCK AND CO., INC, P O BOX 2000, RAHWAY, NJ, 07065-0907, US

(21) Appl. No.: 12/087,141

(22) PCT Filed: Jan. 19, 2007
(86) PCT No.: PCT/US2007/001375

§ 371 Date: Jun. 26, 2008

Related U.S. Application Data
(60) Provisional application No. 60/761,622, filed on Jan. 24, 2006.

(51) Int. Cl. A61K 31/397; C07D 401/02; C07D 401/14; C07D 403/02; C07D 403/14; A61K 31/506; A61K 31/454; A61K 31/4155; A61K 31/403; A61P 29/00; A61P 13/00; A61P 25/00; A61P 1/00; A61P 11/00; A61K 31/4196; A61K 31/4192; A61K 31/4184; A61K 31/426; A61K 31/4439; A61K 31/5377; C07D 413/02; C07D 413/14; C07D 209/52

(52) U.S. Cl. 514/210.18; 548/452; 548/364.7; 514/406; 514/413; 548/255; 546/200; 514/323; 514/304; 546/126; 548/267.6; 514/383; 548/512; 548/468; 514/414; 514/339; 546/276.7; 514/275; 544/332; 544/144; 514/233.2; 514/397; 548/302.7; 548/217; 514/375; 514/393; 514/322; 514/321; 514/234.5; 514/233.8; 546/199; 546/198; 546/271.7; 546/273.1; 544/137; 544/139

(57) ABSTRACT

The present invention is directed to certain hexahydrpyrrolidinone compounds which are useful as neurokinin-1 (NK-1) receptor antagonists, and inhibitors of tachykinin and in particular substance P. The invention is also concerned with pharmaceutical formulations comprising these compounds as active ingredients and the use of the compounds and their formulations in the treatment of certain disorders, including emesis, urinary incontinence, depression, and anxiety.

### BACKGROUND OF THE INVENTION

Substance P is a naturally occurring undecapeptide belonging to the tachykinin family of peptides, the latter being so-named because of their prompt contractile action on extravascular smooth muscle tissue. The tachykinins are distinguished by a conserved carboxyl-terminal sequence. In addition to substance P, the known mammalian tachykinins include neurokinin A and neurokinin B. The current nomenclature designates the receptors for substance P, neurokinin A, and neurokinin B as neurokinin-1 (NK-1), neurokinin-2 (NK-2), and neurokinin-3 (NK-3), respectively.

Tachykinin, and in particular substance P, antagonists are useful in the treatment of clinical conditions which are characterized by the presence of an excess of tachykinin, in particular substance P, activity, including disorders of the central nervous system, nociception and pain, gastrointestinal disorders, disorders of bladder function and respiratory diseases.

### SUMMARY OF THE INVENTION

The present invention is directed to certain hydropyrrolizinone compounds which are useful as neurokinin-1 (NK-1) receptor antagonists, and inhibitors of tachykinin and in particular substance P. The invention is also concerned with pharmaceutical formulations comprising these compounds as active ingredients and the use of the compounds and their formulations in the treatment of certain disorders, including emesis, urinary incontinence, depression, and anxiety.

### DETAILED DESCRIPTION OF THE INVENTION

The present invention is directed to compounds of formulae I, II and III:

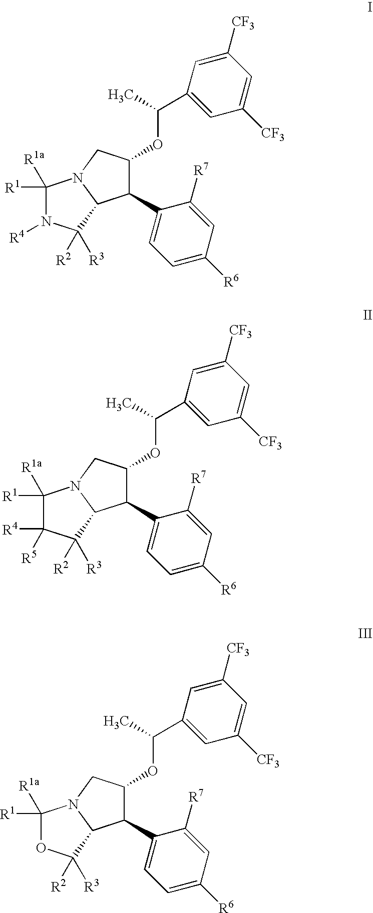

In one aspect the invention is directed to compounds of the formulae I, II and III

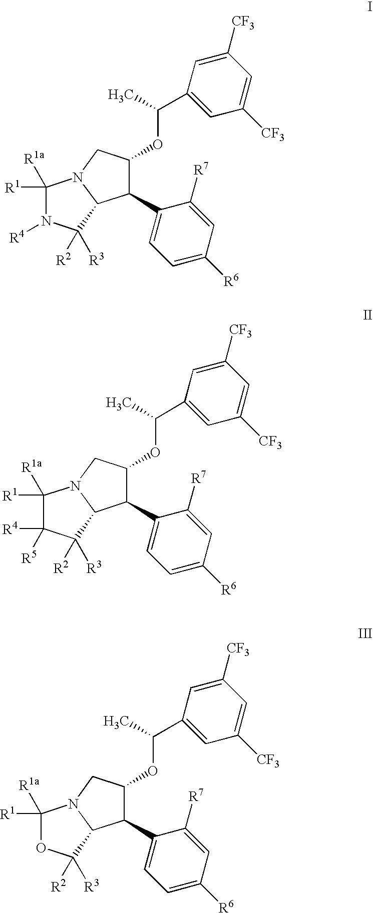

wherein:
R¹ and R^{1a} are each hydrogen or together with the carbon atom to which they are attached form a carbonyl;
Each R² is selected from the group consisting of:

(1) hydrogen,

(2) NH₂, and

(3) CH₃;

R³ are each independently selected from the group consisting of:
(1) hydrogen,
(2) hydroxyl,
(3) NH₂,
(4) N(CH₃)₂;
(5) NH—C(O)—C(CH₃)₂—NH₂,
(6) NH—C(O)—CF₃,
(7) C₁₋₆alkyl,
(8) C₁₋₆alkyl-O—C₁₋₆alkyl, and
(9) -A1, wherein A1 is a heteroaryl or heterocycle of 5 or 6 atoms wherein the heteroaryl or heterocyle contains 1, 2 or 3 heteroatoms selected from the group consisting of O, N and S, and wherein the heteroaryl or heterocylce is optionally substituted with a group selected from methyl, oxo and hydroxyl,
(10) —NH-A1
(11) —NH—CH₂-A1;
(12) CO₂Me, and
(13) CO₂H;
   R⁴ and R⁵ are each independently selected from a group consisting of:
(1) hydrogen,
(2) hydroxy,
(3) hydroxyC₁₋₃alkyl,
(4) C₁₋₃alkyl,
(5) —C(O)—O—CH₃,
(6) NH₂,
(7) aminoC₁₋₃alkyl,
(8) N(R⁹)(R¹⁰),
(9) A2, wherein A2 is selected from the group consisting of

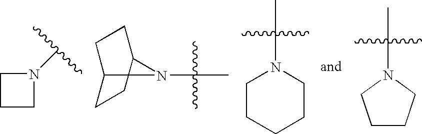

wherein A2 is optionally substituted with a group selected from hydroxyl, methyl, COOH, —COO—C₁₋₄alkyl and

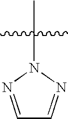

(10) A3, wherein A3 is a heteraromatic or heterocyclic ring of 5 or 6 atoms, wherein 1, 2, or 3 of the atoms is a heteroatom selected from N, S or O, and wherein at least one of the heteroatoms is a N, and wherein the heteroaryl or heterocycle is optionally substituted with a group selected from methyl, oxo, hydroxyl, —CH₃—NH₂ and —CH₃—N(CH₃)₂,
(11) C₁₋₃-A2, and
(12) C₁₋₃-A3,
or R⁴ and R⁵ together with the carbon to which they are attached form a carbonyl;
   R⁶ and R⁷ are each independently selected from a group consisting of:

(1) hydrogen,

(2) halo, and

(3) methyl;

R⁹ and R¹⁰ are each selected from the group consisting of

(1) hydrogen,

(2) methyl,

(3) A4, wherein A4 is a heteroaryl or heterocycle of 5 or 6 atoms wherein the heteroaryl or heterocyle contains 1, 2 or 3 heteroatoms selected from the group consisting of O, N and S, and wherein

the heteroaryl or heterocycle is optionally substituted with a group selected from methyl, oxo, hydroxyl, —CH₃—NH₂ and —CH₃—N(CH₃)₂, and

(4) —C₁₋₃alkyl-A4,

or a pharmaceutically acceptable salts thereof and individual enantiomers and diastereomers thereof.

Within this aspect there is a genus of compounds of formula II

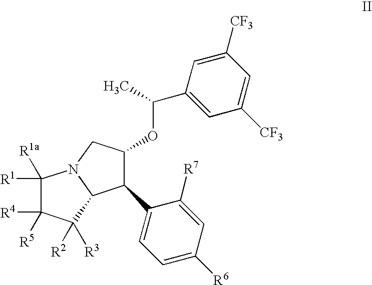

Within this aspect there is a genus of compounds wherein

A1 is selected from the group consisting of

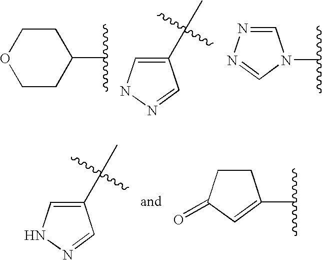

Within this aspect there is a genus of compounds wherein

R³ is selected from the group consisting of

(1) NH₂, and

(2) —NH-A1, wherein A1 is selected from the group consisting of

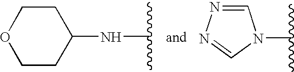

and A1 is optionally substituted with a group selected from methyl, oxo and hydroxyl;

Within this aspect there is a genus of compounds wherein

A3 and A4 are each selected from the group consisting of

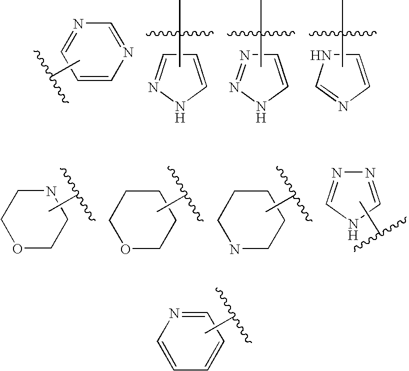

wherein A4 is optionally substituted with a substituent selected from the group consisting of hydroxyl, oxo, methyl, —CH₃—NH₂ and —CH₃—N(CH₃)₂.

Within this aspect there is a genus of compounds wherein

R⁴ and R⁵ are each independently selected from a group consisting of:

(1) hydroxy,

(2) NH₂, and

(3) N(R⁹)(R¹⁰)

Within this aspect there is a genus of compounds wherein

R⁶ and R⁷ are each independently selected from a group consisting of:

(1) hydrogen,

(2) fluoro, and

(3) methyl.

Within this aspect there is a genus of compounds wherein

R⁹ and R¹⁰ are each selected from the group consisting of

(1) hydrogen, and

(2) -A4, wherein A4 is selected from the group consisting of

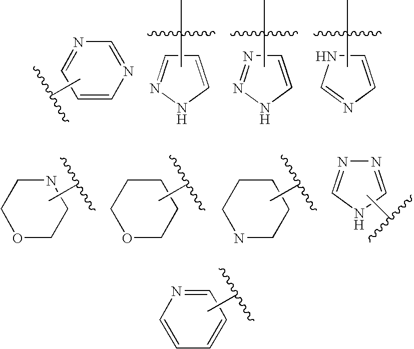

wherein A4 is optionally substituted with a substituent selected from the group consisting of hydroxyl, oxo, methyl, —CH₃—NH₂ and —CH₃—N(CH₃)₂.

Within this aspect there is a genus of compounds wherein

Each R² is selected from the group consisting of:

(1) hydrogen, and

(2) CH₃.

Within this aspect there is a genus of compounds wherein

R⁶ is fluoro and R⁷ is methyl.

Within this aspect there is a genus of compounds of formula II wherein

R¹ and R^{1a} are each hydrogen or together with the carbon atom to which they are attached form a carbonyl;
Each R² is selected from the group consisting of:

(1) hydrogen, and

(2) CH₃;

R³ is selected from the group consisting of (1) NH₂, and

(2) —NH-A1, wherein A1 is selected from the group consisting of

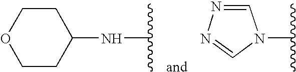

and A1 is optionally substituted with a group selected from methyl, oxo and hydroxyl;

R3 is selected from the group consisting of

(1) NH₂, and

(2) —NH-A1, wherein A1 is selected from the group consisting of

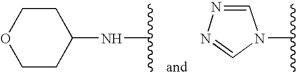

and A1 is optionally substituted with a group selected from methyl, oxo and hydroxyl;

R⁴ and R⁵ are each independently selected from a group consisting of:

(1) hydroxy,

(2) NH₂, and

(3) N(R⁹)(R¹⁰).

R⁶ and R⁷ are each independently selected from a group consisting of:

(1) hydrogen,

(2) fluoro, and

(3) methyl;

R⁹ and R¹⁰ are each selected from the group consisting of

(1) hydrogen,

(2) methyl,

(3) A4, wherein A1 is a heteroaryl or heterocycle of 5 or 6 atoms wherein the heteroaryl or heterocyle contains 1, 2 or 3 heteroatoms selected from the group consisting of O, N and S, and wherein the heteroaryl or heterocylce is optionally substituted with a group selected from methyl, oxo, hydroxyl, —CH₃—NH₂ and —CH₃—N(CH₃)₂, and

(4) —C₁₋₃allyl-A4,

or a pharmaceutically acceptable salts thereof and individual enantiomers and diastereomers thereof.

Within this aspect there is a genus of compounds wherein

of formula I or III
R¹ and R^{1a} are each hydrogen or together with the carbon atom to which they are attached form a carbonyl;
Each R² is selected from the group consisting of:

(1) hydrogen, and

(2) CH₃;

R³ are each independently selected from the group consisting of:

(1) hydrogen,

(2) hydroxyl,

(3) C₁₋₆alkyl, and

(4) C₁₋₆alkyl-O—C₁₋₆alkyl;

R⁴ and R⁵ are each independently selected from a group consisting of:

(1) hydroxy,

(2) NH₂, and

(3) N(R⁹)(R¹⁰).

R⁶ and R⁷ are each independently selected from a group consisting of:

(1) hydrogen,

(2) fluoro) and

(3) methyl;

R⁹ and R¹⁰ are each selected from the group consisting of

(1) hydrogen,

(2) methyl,

(3) A4, wherein A1 is a heteroaryl or heterocycle of 5 or 6 atoms wherein the heteroaryl or heterocyle contains 1, 2 or 3 heteroatoms selected from the group consisting of O, N and S, and wherein the heteroaryl or heterocylce is optionally substituted with a group selected from methyl, oxo, hydroxyl, —CH₃—NH₂ and —CH₃—N(CH₃)₂, and

(4) —C₁₋₃alkyl-A4,

or a pharmaceutically acceptable salts thereof and individual enantiomers and diastereomers thereof.

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

The term “cycloalkyl” means carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyl include cyclopropyl, cyclobutyl, cyclopentyl, cyclohexyl, decahydronaphthatene, adamantane, indanyl, indenyl, fluorenyl, 1,2,3,4-tetrahydronaphalene and the like. Similarly, “cycloalkenyl” means carbocycles containing no heteroatoms and at least one non-aromatic C—C double bond, and include mono-, bi- and tricyclic partially saturated carbocycles, as well as benzofused cycloalkenes. Examples of cycloalkenyl include cyclohexenyl, indenyl, and the like.

The term “aryl” unless specifically stated otherwise includes multiple ring systems as well as single ring systems such as, for example, phenyl or naphthyl.

The term heteroaryl include, for example, pyridinyl, quinolinyl, isoquinolinyl, pyridazinyl, pyrimidinyl, pyrazinyl, quinoxalinyl, furyl, benzofuryl, dibenzofuryl, thienyl, benzthienyl, pyrrolyl, indolyl, pyrazolyl, indazolyl, oxazolyl, isoxazolyl, thiazolyl, isothiazolyl, imidazolyl, benzimidazolyl, oxadiazolyl, thiadiazolyl, triazolyl, tetrazolyl.

The term heterocycle includes, for example, azetidinyl, pyrrolidinyl, piperidinyl, piperazinyl, morpholinyl, tetrahydrofuranyl, imidazolinyl, and thiomorpholinyl.

Specific embodiments of the present invention include a compound which is selected from the group consisting of the subject compounds of the Examples herein and pharmaceutically acceptable salts thereof and individual enantiomers and diastereomers thereof.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. Formula I shows the structure of the class of compounds without preferred stereochemistry. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue The racemic mixture of the compounds can also be separated directly by chromatographic methods utilizing chiral stationary phases, which methods are well known in the art. Alternatively, any enantiomer of a compound may be obtained by stereoselective synthesis using optically pure starting materials or reagents of known configuration by methods well known in the art.

There are several acceptable methods of naming the compounds discussed herein.

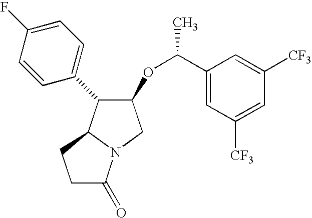

For example, the above compound can be named “(6R,7S,7aS)-6-{(1R)-1-[3,5-bis(trifluoromethylophenyl]ethoxy}-7-(4-fluorophenyl)hexahydro-3H-pyrrolizin-3-one. The core structure may be generally referred to as hexahydro-3H-pyrrolizin-3-one, hexahydropyrrolizinone perhydro-3H-pyrrolizin-3-one, perhydropyrrolizinone compounds.

As appreciated by those of skill in the art, halo or halogen as used herein are intended to include fluoro, chloro, bromo and iodo. Similarly, C₁₋₆, as in C₁₋₆alkyl is defined to identify the group as having 1, 2, 3, 4, 5 or 6 carbons in a linear or branched arrangement, such that C₁₋₈alkyl specifically includes methyl, ethyl, n-propyl, iso-propyl, n-butyl, iso-butyl, tert-butyl, pentyl, and hexyl. A group which is designated as being independently substituted with substituents may be independently substituted with multiple numbers of such substituents.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids. It will be understood that, as used herein, references to the compounds of the present invention are meant to also include the pharmaceutically acceptable salts.

Exemplifying the invention is the use of the compounds disclosed in the Examples and herein. Specific compounds within the present invention include a compound which selected from the group consisting of the compounds disclosed in the following Examples and pharmaceutically acceptable salts thereof and individual diastereomers thereof.

The compounds of the present invention are useful in the prevention and treatment of a wide variety of clinical conditions which are characterized by the presence of an excess of tachykinin, in particular substance P, activity. Thus, for example, an excess of tachykinin, and in particular substance P, activity is implicated in a variety of disorders of the central nervous system. Such disorders include mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalised anxiety disorders; schizophrenia and other psychotic disorders, for example, schizophreniform disorders, schizoaffective disorders, delusional disorders, brief psychotic disorders, shared psychotic disorders and psychotic disorders with delusions or hallucinations; delirium, dementia, and amnestic and other cognitive or neurodegenerative disorders, such as Alzheimer's disease, senile dementia, dementia of the Alzheimer's type, vascular dementia, and other dementias, for example, due to HIV disease, head trauma, Parkinson's disease, Huntington's disease, Pick's disease, Creutzfeldt-Jakob disease, or due to multiple aetiologies; Parkinson's disease and other extra-pyramidal movement disorders such as medication-induced movement disorders, for example, neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremour; substance-related disorders arising from the use of alcohol, amphetamines (or amphetamine-like substances) caffeine, cannabis, cocaine, hallucinogens, inhalants and aerosol propellants, nicotine, opioids, phenylglycidine derivatives, sedatives, hypnotics, and anxiolytics, which substance-related disorders include dependence and abuse, intoxication, withdrawal, intoxication delerium, withdrawal delerium, persisting dementia, psychotic disorders, mood disorders, anxiety disorders, sexual dysfunction and sleep disorders; epilepsy; Down's syndrome; demyelinating diseases such as MS and ALS and other neuropathological disorders such as peripheral neuropathy, for example diabetic and chemotherapy-induced neuropathy, and postherpetic neuralgia, trigeminal neuralgia, segmental or intercostal neuralgia and other neuralgias; and cerebral vascular disorders due to acute or chronic cerebrovascular damage such as cerebral infarction, subarachnoid haemorrhage or cerebral oedema.

Tachykinin, and in particular substance P, activity is also involved in nociception and pain. The compounds of the present invention will therefore be of use in the prevention or treatment of diseases and conditions in which pain predominates, including soft tissue and peripheral damage, such as acute trauma, osteoarthritis, rheumatoid arthritis, musculo-skeletal pain, particularly after trauma, spinal pain, myofascial pain syndromes, headache, episiotomy pain, and burns; deep and visceral pain, such as heart pain, muscle pain, eye pain, orofacial pain, for example, odontalgia, abdominal pain, gynaecological pain, for example, dysmenorrhoea, and labour pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; pain associated with carcinoma, often referred to as cancer pain; central nervous system pain, such as pain due to spinal cord or brain stein damage; low back pain; sciatica; ankylosing spondylitis, gout; and scar pain.

Tachykinin, and in particular substance P, antagonists may also be of use in the treatment of respiratory diseases, particularly those associated with excess mucus secretion, such as chronic obstructive airways disease, bronchopneumonia, chronic bronchitis, cystic fibrosis and asthma, adult respiratory distress syndrome, and bronchospasm; inflammatory diseases such as inflammatory bowel disease, psoriasis, fibrositis, osteoarthritis, rheumatoid arthritis, pruritis and sunburn; allergies such as eczema and rhinitis; hypersensitivity disorders such as poison ivy; ophthalmic diseases such as conjunctivitis, vernal conjunctivitis, and the like; ophthalmic conditions associated with cell proliferation such as proliferative vitreoretinopathy; cutaneous diseases such as contact dermatitis, atopic dermatitis, urticaria, and other eczematoid dermatitis. Tachykinin, and in particular substance P, antagonists may also be of use in the treatment of neoplasms, including breast tumours, neuroganglioblastomas and small cell carcinomas such as small cell lung cancer.

Tachykinin, and in particular substance P, antagonists may also be of use in the treatment of gastrointestinal (GI) disorders, including inflammatory disorders and diseases of the GI tract such as gastritis, gastroduodenal ulcers, gastric carcinomas, gastric lymphomas, disorders associated with the neuronal control of viscera, ulcerative colitis, Crohn's disease, irritable bowel syndrome and emesis, including acute, delayed or anticipatory emesis such as emesis induced by chemotherapy, radiation, toxins, viral or bacterial infections, pregnancy, vestibular disorders, for example, motion sickness, vertigo, dizziness and Meniere's disease, surgery, migraine, variations in intercranial pressure, gastro-oesophageal reflux disease, acid indigestion, over indulgence in food or drink, acid stomach, waterbrash or regurgitation, heartburn, for example, episodic, nocturnal or meal-induced heartburn, and dyspepsia.

Tachykinin, and in particular substance P, antagonists may also be of use in the treatment of a variety of other conditions including stress related somatic disorders; reflex sympathetic dystrophy such as shoulder/hand syndrome; adverse immunological reactions such as rejection of transplanted tissues and disorders related to immune enhancement or suppression such as systemic lupus erythematosus; plasma extravasation resulting from cytokine chemotherapy, disorders of bladder function such as cystitis, bladder detrusor hyper-reflexia, frequent urination and urinary incontinence, including the prevention or treatment of overactive bladder with symptoms of urge urinary incontinence, urgency, and frequency; fibrosing and collagen diseases such as scleroderma and eosinophilic fascioliasis; disorders of blood flow caused by vasodilation and vasospastic diseases such as angina, vascular headache, migraine and Reynaud's disease; and pain or nociception attributable to or associated with any of the foregoing conditions, especially the transmission of pain in migraine. The compounds of the present invention are also of value in the treatment of a combination of the above conditions, in particular in the treatment of combined post-operative pain and post-operative nausea and vomiting.

The compounds of the present invention are particularly useful in the prevention or treatment of emesis, including acute, delayed or anticipatory emesis, such as emesis induced by chemotherapy, radiation, toxins, pregnancy, vestibular disorders, motion, surgery, migraine, and variations in intercranial pressure. For example, the compounds of the present invention are of use optionally in combination with other antiemetic agents for the prevention of acute and delayed nausea and vomiting associated with initial and repeat courses of moderate or highly emetogenic cancer chemotherapy, including high-dose cisplatin. Most especially, the compounds of the present invention are of use in the treatment of emesis induced by antineoplastic (cytotoxic) agents, including those routinely used in cancer chemotherapy, and emesis induced by other pharmacological agents, for example, rolipram. Examples of such chemotherapeutic agents include alkylating agents, for example, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; and cytotoxic antibiotics. Particular examples of chemotherapeutic agents are described, for instance, by D. J. Stewart in Nausea and Vomiting: Recent Research and Clinical Advances, Eds J. Kucharczyk et al, CRC Press Inc., Boca Raton, Fla., USA (1991) pages 177-203, especially page 188. Commonly used chemotherapeutic agents include cisplatin, dacarbazine (DTIC), dactinomycin, mechlorethamine, streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin and chlorambucil [R. J. Gralla et al in Cancer Treatment Reports (1984) 68(1), 163-172]. A further aspect of the present invention comprises the use of a compound of the present invention for achieving a chronobiologic (circadian rhythm phase-shifting) effect and alleviating circadian rhythm disorders in a mammal. The present invention is further directed to the use of a compound of the present invention for blocking the phase-shifting effects of light in a mammal.

The present invention is further directed to the use of a compound of the present invention or a pharmaceutically acceptable salt thereof, for enhancing or improving sleep quality as well as preventing and treating sleep disorders and sleep disturbances in a mammal. In particular, the present invention provides a method for enhancing or improving sleep quality by increasing sleep efficiency and augmenting sleep maintenance. In addition, the present invention provides a method for preventing and treating sleep disorders and sleep disturbances in a mammal which comprising the administration of a compound of the present invention or a pharmaceutically acceptable salt thereof. The present invention is useful for the treatment of sleep disorders, including Disorders of Initiating and Maintaining Sleep (insomnias) (“DIMS”) which can arise from psychophysiological causes, as a consequence of psychiatric disorders (particularly related to anxiety), from drugs and alcohol use and abuse (particularly during withdrawal stages), childhood onset DIMS, nocturnal myoclonus, fibromyalgia, muscle pain, sleep apnea and restless legs and non specific REM disturbances as seen in ageing.

The particularly preferred embodiments of the instant invention are the treatment of emesis, urinary incontinence, depression or anxiety by administration of the compounds of the present invention to a subject (human or animal) in need of such treatment.

The present invention is directed to a method for the manufacture of a medicament for antagonizing the effect of substance P at its receptor site or for the blockade of neurokinin-1 receptors in a mammal comprising combining a compound of the present invention with a pharmaceutical carrier or diluent. The present invention is further directed to a method for the manufacture of a medicament for the treatment of a physiological disorder associated with an excess of tachykinins in a mammal comprising combining a compound of the present invention with a pharmaceutical carrier or diluent.

The present invention also provides a method for the treatment or prevention of physiological disorders associated with an excess of tachykinins, especially substance P, which method comprises administration to a patient in need thereof of a tachykinin reducing amount of a compound of the present invention or a composition comprising a compound of the present invention. As used herein, the term “treatment” or “to treat” refers to the administration of the compounds of the present invention to reduce, ameliorate, or eliminate either the symptoms or underlying cause of the noted disease conditions, in a subject (human or animal) that suffers from that condition or displays clinical indicators thereof. The term “prevention” or “to prevent” refers to the administration of the compounds of the present invention to reduce, ameliorate, or eliminate the risk or likelihood of occurrence of the noted disease conditions, in a subject (human or animal) susceptible or predisposed to that condition.

The compounds of this invention are useful for antagonizing tachykinins, in particular substance P in the treatment of gastrointestinal disorders, central nervous system disorders, inflammatory diseases, pain or migraine and asthma in a mammal in need of such treatment. This activity can be demonstrated by the following assays.

Receptor Expression in COS: To express the cloned human neurokinin-1 receptor (NK1R) transiently in COS, the cDNA for the human NK1R was cloned into the expression vector pCDM9 which was derived from pCDM8 (INVITROGEN) by inserting the ampicillin resistance gene (nucleotide 1973 to 2964 from BLUESCRIPT SK+) into the Sac II site. Transfection of 20 ug of the plasmid DNA into 10 million COS cells was achieved by electroporation in 800 ul of transfection buffer (135 mM NaCl, 1.2 mM CaCl₂, 1.2 mM MgCl₂, 2.4 mM K₂HPO₄, 0.6 mM KH₂PO₄, 10 mM glucose, 10 mM HEPES pH 7.4) at 260 V and 950 uF using the IBI GENEZAPPER (IBI, New Haven, Conn.). The cells were incubated in 10% fetal calf serum, 2 mM glutamine, 100 U/ml penicillin-streptomycin, and 90% DMEM media (GIBCO, Grand Island, N.Y.) in 5% CO₂ at 37° C. for three days before the assay.

Stable Expression in CHO: To establish a stable cell line expressing the cloned human NK1R, the cDNA was subcloned into the vector pRcCMV (INVITROGEN). Transfection of 20 ug of the plasmid DNA into CHO cells was achieved by electroporation in 800 ul of transfection buffer supplemented with 0.625 mg/ml Herring sperm DNA at 300 V and 950 uF using the IBI GENEZAPPER (IBI). The transfected cells were incubated in CHO media [10% fetal calf serum, 100 U/ml penicillin-streptomycin, 2 mM glutamine, 1/500 hypoxanthine-thymidine (ATCC), 90% IMDM media (JRH BIOSCIENCES, Lenexa, Kans.), 0.7 mg/ml G418 (GIBCO)] in 5% CO₂ at 37° C. until colonies were visible. Each colony was separated and propagated. The cell clone with the highest number of human NK1R was selected for subsequent applications such as drug screening.

Assay Protocol using COS or CHO: The binding assay of human NK1R expressed in either COS or CHO cells is based on the use of ¹²⁵I-substance P(¹²⁵I—SP, from DU PONT, Boston, Mass.) as a radioactively labeled ligand which competes with unlabeled substance P or any other ligand for binding to the human NK1R. Monolayer cell cultures of COS or CHO were dissociated by the non-enzymatic solution (SPECIALTY MEDIA, Lavallette, N.J.) and resuspended in appropriate volume of the binding buffer (50 mM Tris pH 7.5, 5 mM MnCl₂, 150 mM NaCl, 0.04 mg/ml bacitracin, 0.004 mg/ml leupeptin, 0.2 mg/ml BSA, 0.01 mM phosphoramidon) such that 200 ul of the cell suspension would give rise to about 10,000 cpm of specific ¹²⁵I—SP binding (approximately 50,000 to 200,000 cells). In the binding assay, 200 ul of cells were added to a tube containing 20 ul of 1.5 to 2.5 nM of ¹²⁵I—SP and 20 ul of unlabeled substance P or any other test compound. The tubes were incubated at 4° C. or at room temperature for 1 hour with gentle shaking. The bound radioactivity was separated from unbound radioactivity by GF/C filter (BRANDEL, Gaithersburg, Md.) which was pre-wetted with 0.1% polyethylenimine. The filter was washed with 3 ml of wash buffer (50 mM Tris pH 7.5, 5 mM MnCl₂, 150 mM NaCl) three times and its radioactivity was determined by gamma counter. The activation of phospholipase C by NK1R may also be measured in CHO cells expressing the human NK1R by determining the accumulation of inositol monophosphate which is a degradation product of IP₃. CHO cells are seeded in 12-well plate at 250,000 cells per well. After incubating in CHO media for 4 days, cells are loaded with 0.025 uCi/ml of ³H-myoinositol by overnight incubation. The extracellular radioactivity is removed by washing with phosphate buffered saline. LiCl is added to the well at final concentration of 0.1 mM with or without the test compound, and incubation is continued at 37° C. for 15 min. Substance P is added to the well at final concentration of 0.3 nM to activate the human NK1R. After 30 min of incubation at 37° C., the media is removed and 0.1 N HCl is added. Each well is sonicated at 4° C. and extracted with CHCl₃/methanol (1:1). The aqueous phase is applied to a 1 ml Dowex AG 1X8 ion exchange column. The column is washed with 0.1 N formic acid followed by 0-025 M ammonium formate-0.1 N formic acid. The inositol monophosphate is eluted with 0.2 M ammonium formate-0.1 N formic acid and quantitated by beta counter. In particular, the intrinsic tachykinin receptor antagonist activities of the compounds of the present invention may be demonstrated by these assays. The compounds of the following examples have activity in the aforementioned assays in the range of 0.05 nM to 10 μM. The activity of the present compounds may also be demonstrated by the assay disclosed by Lei, et al., British J. Pharmacol., 105, 261-262 (1992).

According to a further or alternative aspect, the present invention provides a compound of the present invention for use as a composition that may be administered to a subject in need of a reduction of the amount of tachykinin or substance P in their body.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

The compositions containing compounds of the present invention may be presented in unit dosage form and may be prepared by any of the methods well known in the art of pharmacy. The term “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets or capsules for oral administration, single dose vials for injection, or suppositories for rectal administration. This list of unit dosage forms is not intended to be limiting in any way, but merely to represent typical examples in the pharmacy arts of unit dosage forms. The compositions containing compounds of the present invention may also be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient.

By “pharmaceutically acceptable” it is meant the carrier, diluent or excipient must be compatible with the other ingredients of the formulation and not deleterious to the recipient thereof.

The terms “administration of” or “administering a” compound should be understood to mean providing a compound of the invention to the individual in need of treatment in a form that can be introduced into that individuals body in a therapeutically useful form and therapeutically effective amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories. The term “therapeutically effective amount” refers to a sufficient quantity of the compounds of the present invention, in a suitable composition, and in a suitable dosage form to treat or prevent the noted disease conditions.

The compounds of the present invention may be administered in combination with another substance that has a complimentary effect to the tachykinin and substance P inhibitors of the present invention. Accordingly, in the prevention or treatment of emesis, a compound of the present invention may be used in conjunction with other anti-emetic agents, especially 5HT₃ receptor antagonists, such as ondansetron, granisetron, tropisetron, palenosetron and zatisetron, a corticosteroid, such as dexamethasone, or GABA_{B} receptor agonists, such as baclofen. Likewise, for the prevention or treatment of migraine a compound of the present invention may be used in conjunction with other anti-migraine agents, such as ergotamines or 5HT₁ agonists, especially sumatriptan, naratriptan, zolmatriptan or rizatriptan.

It will be appreciated that for the treatment of depression or anxiety, a compound of the present invention may be used in conjunction with other anti-depressant or anti-anxiety agents, such as norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), α-adrenoreceptor antagonists, atypical anti-depressants, benzodiazepines, 5-HT_{1A} agonists or antagonists, especially 5-HT_{1A} partial agonists, corticotropin releasing factor (CRF) antagonists, and pharmaceutically acceptable salts thereof. For the treatment or prevention of eating disorders, including obesity, bulimia nervosa and compulsive eating disorders, a compound of the present invention may be used in conjunction with other anorectic agents. It will be appreciated that for the treatment or prevention of pain or nociception or inflammatory diseases, a compound of the present invention may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent.

It will be appreciated that when using any combination described herein, both the compound of the present invention and the other active agent(s) will be administered to a patient, within a reasonable period of time. The compounds may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, one active component may be administered as a tablet and then, within a reasonable period of time, the second active component may be administered either as an oral dosage form such as a tablet or a fast-dissolving oral dosage form. By a “fast dissolving oral formulation” is meant, an oral delivery form which when placed on the tongue of a patient, dissolves within about 10 seconds. By “reasonable period of time” is meant a time period that is not in excess of about 1 hour. That is, for example, if the first active component is provided as a tablet, then within one hour, the second active component should be administered, either in the same type of dosage form, or another dosage form which provides effective delivery of the medicament.

The compounds of this invention may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician.

In the treatment of the conditions associated with an excess of tachykinins, a suitable dosage level of the compounds of the present invention, or pharmaceutically acceptable salts thereof, is about 0.001 to 50 mg/kg per day, in particular about 0.01 to about 25 mg/kg, such as from about 0.05 to about 10 mg/kg per day. The dosage range will generally be about 0.5 to 1000 mg per patient per day, which may be administered in single or multiple doses. Preferably, the dosage range will be about 0.5 mg to 500 mg per patient per day; more preferably about 0.5 mg to 200 mg per patient per day; and even more preferably about 5 mg to 50 mg per patient per day. Specific dosages of the compounds of the present invention, or pharmaceutically acceptable salts thereof, for administration include 1 mg, 5 mg, 10 mg, 30 mg, 100 mg, and 500 mg. Pharmaceutical compositions of the present invention may be provided in a formulation comprising about 0.5 mg to 1000 mg active ingredient; more preferably comprising about 0.5 mg to 500 mg active ingredient; or 0.5 mg to 250 mg active ingredient; or 1 mg to 100 mg active ingredient. Specific pharmaceutical compositions for treatment or prevention of excess tachykinins comprise about 1 mg, 5 mg, 10 mg, 30 mg, 100 mg, and 500 mg of active ingredient.

Several methods for preparing the compounds of this invention are illustrated in the following Examples. Starting materials and the requisite intermediates are in some cases commercially available, or can be prepared according to literature procedures or as illustrated herein. All ¹H NMR spectra were obtained on instrumentation at a field strength of 400 or 500 MHz.

The following examples are provided for the purpose of further illustration only and are not intended to be limitations on the disclosed invention.

### EXAMPLE 1

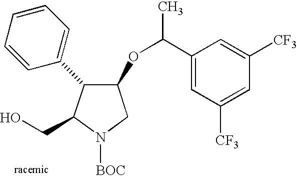

### tert-Butyl 4-{1-[3,5-bis(trifluoromethyl)phenyl]ethoxy}-2-(hydroxymethyl)-3-phenylpyrrolidine-1-carboxylate

### Step A: tert-Butyl 2-(hydroxymethyl)-2,5-dihydro-1H-pyrrole-1-carboxylate

To a solution of 10 g (44 mmol) 1-tert-butyl 2-methyl 2,5-dihydro-1H-pyrrole-1,2-dicarboxylate (prepared according to the procedure of Sturmer, R., Schafer, B., Wolfart, V., Stahr, H., Kazmaier, U., Helmchen, G.; Synthesis 2001, (1), p. 46-48) in 150 mL dry THF under nitrogen atmosphere at −78° C. was added dropwise over 30 min 100 mL (100 mmol) of a 11.0M solution of DIBAL in cyclohexane. The reaction mixture was stirred at −78° C. for 15 min then warmed to room temperature. Upon completion of the reaction (as monitored by TLC), the reaction mixture was quenched with excess water and transferred to a separatory funnel. The reaction mixture was extracted with EtOAc (2×200 mL) then methylene chloride (100 mL). The combined organic extracts were dried over anhydrous sodium sulfate, filtered and evaporated under vacuum. The resulting thick yellow oil was purified by silica gel chromatography eluting with gradient hexanes/EtOAc (9/1) to hexanes/EtOAc (2/8). The product fractions were combined and evaporated under vacuum to give 16.3 g of the title compound. ¹H (500 MHz, CDCl₃) δ 5.82 (dd, J=1.3, 4.6 Hz, 1H); 5.66-5.62 (m, 1H); 4.75 (br s, 1H); 4.20 (dd, J=1.9, 15.6 Hz, 1H); 4.10 (ddd, J=2.0, 5.5, 15.6 Hz, 1H); 3.80 (dd, J=2.1, 11.4 Hz, 1H); 3.58 (dd, J=7.5, 11.4 Hz, 1H), 1.52 (s, 9H) ppm.

### Step B: tert-Butyl 2-(hydroxymethyl)-6-oxa-3-azabicyclo[3.1.0]hexane-3-carboxylate

To a solution 16.3 g (81.9 mmol) product Step A in 500 mL methylene chloride was added 163.7 mmol (2 equiv.) MCPBA. The reaction mixture was stirred at ambient T for 24 hr. The reaction mixture was quenched with excess Ca(OH)₂ and stirred vigorously for 30 min. The reaction mixture was filtered, washed with methylene chloride and the solvent of the filtrate removed under vacuum. The residue was purified by chromatography on silica gel eluting with a hexanes/EtOAc (100/0 to 10/90) gradient system to provide two isomeric products; 6.57 g of the major less polar isomer 1 (syn isomer). ¹H NMR (500 MHz, CDCl₃): δ 3.98 (d, J=10.8 Hz, 1H); 3.94-3.82 (m, 2H); 3.77 (br s, 1H); 3.74 (d, J=13 Hz, 1H); 3.62 (s, 1H); 3.42 (d, J=13 Hz, 1H); 1.46 (s, 9H). ¹³C (125 MHz, CDCl₃) 158.02, 64.19, 61.90, 58.58, 53.75, 49.71, 28.57 ppm. Also obtained 3.44 g of the minor more polar isomer 2 (anti isomer). ¹H NMR (500 MHz, CDCl₃): δ 4.17 (t, J=5.2 Hz, 0.5H), 4.03 (t, J=4.2 Hz, 0.5H), 3.88 (d, J=13 Hz, 0.5H), 3.86-3.74 (m, 3H), 3.70-3.65 (m, 1H), 3.60 (d, J=3.0 Hz, 0.5H), 3.37 (dd, J=1.2, 13 Hz, 1H), 1.45 (br s, 9H). ¹³C (125 MHz, CDCl₃): δ 156.11 (154.96), 80.88 (80.62), 62.86 (62.37), 60.18 (60.07), 58.08 (57.29), 55.35 (54.89), 47.81 (47.75), 28.69 (28.62).

### Step C: tert-Butyl 2-({[tert-butyl(dimethyl)silyl]oxy}methyl)-6-oxa-3-azabicyclo[3.1.0]hexane-3-carboxylate

To a solution of 6.56 g (30.5 mmol) of major isomer 1 of intermediate from Step B in 45 mL dry DMF under nitrogen atmosphere was added 4.17 g (61 mmol) imidazole followed by 4.6 g (30.5 mmol) tert-butylchlorodimethylsilane. The reaction mixture was stirred for 16 hr then diluted with water. The mixture was transferred to a separatory funnel and extracted with ether (3×50 mL). The combined ether extracts were washed with water (2×25 mL), dried over magnesium sulfate filtered and the solvent removed under vacuum. The residue was purified by chromatography on silica gel eluting with a hexanes/EtOAc gradient (95/5 to 40/60) to provide 9.04 g of the title compound. ¹H NMR (500 MHz, CDCl₃): δ 4.33 (br s, 0.5H); 4.15 (br s, 0.5H); 3.90 (t, J=2.5 Hz, 1H); 3.88-3.76 (m, 1H); 3.72 (br d, J=14.2 Hz, 1H); 3.65 (d, J=12.5 Hz, 1H); 3.60-3.52 (m, 1H); 3.46 (d, J=12.5 Hz, 1H); 1.46 (br s, 9H); 0.93 (s, 9H); 0.12 (s, 6H). ppm.

### Step D: tert-Butyl 2-({[tert-butyl(dimethyl)silyl]oxy}methyl)-4-hydroxy-3-phenylpyrrolidine-1-carboxylate (more polar isomer) and tert-Butyl 2-({[tert-butyl(dimethyl)silyl]oxy}methyl)-3-hydroxy-4-phenylpyrrolidine-1-carboxylate (less polar isomer)

To a slurry of 25 mg (0.13 mmol) Cul in 2 mL dry THF cooled to 0° C. in an ice/MeOH bath was added dropwise by syringe 1.31 mL (1.31 mmol) of a 1.0M solution of phenylmagnesium bromide in THF. The resulting mixture was stirred for 10 min. at which time a solution of 250 mg (0.87 mmol) of Intermediate Step C in 1 mL THF was added. The resulting reaction mixture was stirred for 5 hrs at ambient T. To the mixture was quenched by the addition of 2 mL water and extracted with ether 93×10 mL). The combined organic extracts were dried over anhydrous sodium sulfate filtered and the solvent removed under vacuum. The resulting oil was purified by preparative TLC eluting with EtOAc/Hexanes (25/75) to provide two isomeric products. More polar isomer by TLC: 130 mg (37%). ¹H NMR (500 MHz, CDCl₃): δ 7.35 (app t, J=7.5 Hz, 2H), 7.28-7.24 (m, 1H), 7.16 (bd dd, J=7.55, 10.5 Hz, 2H); 4.44 (dd, J=2.4, 10.6 Hz, 1H); 4.24-4.16 (m, 1H); 4.04 (br s, 1H), 3.76-3.70 (m, 1H); 3.67 (d, J=10.6 Hz, 1H); 3.48 (dd, J=12.4, 15.6 Hz, 1H); 3.37 (s, 1H); 1.53 (s, 9H); 0.96 (s, 9H); 0.17 (s, 6H). MS: 408 (M+H), 308 (M+H-Boc). Minor less polar isomer by TLC: 103 mg (34%). ¹H NMR (500 MHz, CDCl₃): δ 7.38-7.35 (m, 2H); 7.34-7.25 (m, 3H); 4.44 (dd, J=8.45, 16 HZ, 1H); 4.24 (dd, J=4.0, 10.9 Hz, 0.5H), 4.10-4.02 (m, 2H); 3.97-3.92 (m, 0.5H); 3.82 (dd, J=8.5, 16.4 Hz, 1H), 3.52-3.36 (m, 3H), 1.52 (s, 3.5H); 1.48 (s, 5.5H), 0.96 (s, 9H); 0.14 (s, 6H). MS: 408 (M+H), 308 (M+H-Boc).

### Step E: tert-Butyl 4-{[3,5-bis(trifluoromethyl)benzoyl]oxy}-2-({[tert-butyl(dimethyl)silyl]oxy}methyl)-3-phenylpylrrolidine-1-carboxylate

To a solution 3.0 g (7.37 mmol) of the more polar isomer of Step D in 60 mL dry methylene chloride under nitrogen atmosphere was added 2.27 mL (16.2 mmol) DIPEA followed by 1.47 mL (8.1 mmol) 3,5-bis(trifluoromethyl)benzoyl chloride. The resulting mixture was stirred at ambient T for 16 hr the partitioned between aq. 1N HCl (5 mL). The layers were separated and the organic layer was washed with sat. aq. sodium bicarbonate (5 mL) then brine (5 mL), dried over anhydr. sodium sulfate, filtered and the solvent removed under vacuum. The residue was purified by chromatography on silica gel eluting with EtOAc/hexanes gradient (0% to 40% EtOAc) to afford 4.52 g of the title compound. ¹H NMR (500 MHz, CDCl₃): δ 8.46 (s, 2H); 8.10 (s, 1H); 7.38 (app t, J=7.4 Hz, 2H); 7.34-7.26 (m, 3H); 5.92-5.45 (br s, 1H); 4.25 (dd, J=6.4, 12.1 Hz, 1H); 4.10 (br s, 1H); 4.08-4.00 (m, 1H); 3.92 (br d, J=7.0 Hz, 2H); 3.80 (br s, 1H); 3.58 (d, 10.7 Hz, 0.5H); 3.48-3.42 (m, 0.5H); 1.53 (s, 9H); 0.90 (s, 5H); 0.88 (s, 4H), 0.07 (s, 2.5H); 0.04 (s, 3.5H) ppm. MS: 670 (M+Na), 548 (M+H-Boc).

### Step F: tert-Butyl 4-({1-[3,5-bis(trifluoromethyl)phenyl]vinyl}oxy)-2-({[tert-butyl(dimethyl)silyl]oxy}methyl)-3-phenylpyrrolidine-1-carboxylate

In a pressure tube was placed a solution of 1.0 g (1.55 mmol) of the intermediate of Step E in 30 mL dry toluene. To this solution was added 6.2 mL (6.2 mmol) of a 1.0 M solution of Petasis reagent in toluene. The pressure tube was purged several times with nitrogen, sealed and heated at 70° C. for 16 hr. The reaction vessel was cooled to RT and filtered through a plug of silica gel eluting with EtOAc/hexanes (20/80) to afford 500 mg (50%) of the title compound. MS: 668 (M+Na), 547 (M+H-Boc).

### Step G: tert-Butyl 4-{1-[3,5-bis(trifluoromethyl)phenyl]ethoxy}-2-({[tert-butyl(dimethyl)silyl]oxy}methyl)-3-phenylpyrrolidine-1-carboxylate

To a solution of 50 mg (0.077 mmol) intermediate step F in 3 mL MeOH was added 5 mg (0.1 by wt) 10% Pd—C. The resulting mixture was degassed and then stirred under hydrogen balloon atmosphere for 3 hr. The reaction was filtered and the solvent of the filtrate removed under vacuum. The residue was purified by prep TLC eluting with EtOAc/Hexanes (1/9). Less polar Diastereomer 1: 24 mg (48%); ¹H NMR (500 MHz, CDCl₃): δ 7.68 (s, 1H); 7.43 (s, 2H); 7.20-7.15 (m, 3H); 7.07 (d, J=6.4 Hz, 2H); 4.38 (q, J=6.2 Hz, 1H); 4.12 (dd, J=6.8, 10.0 Hz, 1H); 4.04-3.91 (m, 2H); 3.70 (br s, 1H); 3.64 (d, 10.5 Hz, 0.5H); 3.52-3.39 (m, 1.5H); 3.30 (dd, J=8.0, 10.3 Hz, 1H); 1.52 (s, 2.5H); 1.48 (s, 6.5H); 1.38 (d, J=6.2 Hz, 3H); 0.91 (s, 9H); 0.12 (s, 6H)ppm. More polar diastereomer 2; 26 mg (52%): ¹H NMR (500 MHz, CDCl₃): δ 7.72 (s, 1H); 7.52 (s, 1H); 7.49 (s, 1H); 7.27-7.21 (m, 3H); 7.07 (d, J=6.4 Hz, 2H); 4.54-4.45 (m, 1H); 4.12 (dd, J=6.8, 10.0 Hz, 1H); 4.04-3.95 (m, 1H); 3.91 (d, J=7.0 Hz, 1H); 3.82-3.78 (m, 0.5H); 3.73 (br s, 0.5H); 3.64 (d, 10.5 Hz, 0.5H); 3.60-3.50 (m, 1.5H); 3.30 (dd, J=8.0, 10.3 Hz, 1H); 1.52 (s, 3.5H); 1.48 (s, 5.5H); 1.40 (d, J=7.6 Hz, 3H); 0.94 (s, 9H); 0.17 (s, 3H); 0.15 (s, 3H)ppm.

### Step H: tert-Butyl 4-{1-[3,5-bis(trifluoromethyl)phenyl]ethoxy}-2-(hydroxymethyl)-3-phenylpyrrolidine-1-carboxylate

The more polar intermediate Step G, 90 mg (0.14 mmol), was dissolved in ˜2 mL (˜2 mmol) of a 1.0M solution of TBAF in THF. The reaction mixture was stirred at RT for 2 hr then the solvent was removed under vacuum. The residue was taken up in methylene chloride (˜50 mL) washed with water (2×5 mL), dried over anhydr. sodium sulfate, filtered and the solvent removed under vacuum The residue was purified by prep. TLC eluting with hexanes/EtOAc (4/6) to afford 68 mg of the title compound. ¹H NMR (500 MHz, CDCl₃): δ 7.72 (s, 1H); 7.46 (s, 2H); 7.27-7.24 (m, 3H); 7.08 (dd, J=2.0, 7.2 Hz, 2H); 4.45 (app q, J=6.3 Hz, 1H); 4.00 (dd, J=2.3, 11.0 Hz, 2H) 3.99 (overlapping br s, 1H); 3.72 (br s, 2H), 3.38 (app t, J=11.2 Hz, 1H), 2.92 (br s, 1H); 1.53 (s, 9H); 1.41 (d, J=6.2 Hz, 3H)ppm. MS: 556 (M+Na), 434 (M+H-Boc).

### EXAMPLE 2

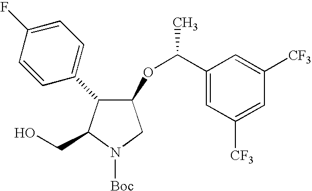

### tert-Butyl (2R,3S,4R)-4-{(1R)-1-[3,5-bis(trifluoromethyl)phenyl]ethoxy}-2-(hydroxymethyl)-3-(4-fluorophenyl)pyrrolidine-1-carboxylate

### Step A: 1-tert-butyl 2-methyl (2R)-2,5-dihydro-1H-pyrrole-1,2-dicarboxylate

(Prepared according to the procedure of Sturmer, R., Schafer, B., Wolfart, V., Stahr, H., Kazmaier, U., Helmchen, G.; Synthesis 2001, (1), p. 46-48) To a mixture of 8 g (35.2 mmol) 1-tert-butyl 2-methyl (2RS)-2,5-dihydro-1H-pyrrole-1,2-dicarboxylate in 200 mL distilled water was added 1.6 g (19.3 mmol) sodium bicarbonate followed by 1.6 g Novozyme 435®. The resulting suspension was heated at 50° C. in an oil bath for 18 hr. The resulting orange mixture was cooled to room temperature and the solids removed by filtration. The solids were washed with water (2×20 mL) and ether. The filtrate was extracted with ether (3×50 mL) and the solvent removed under vacuum to afford the 1-tert-butyl 2-methyl (2R)-2,5-dihydro-1H-pyrrole-2-dicarboxylate.

The 1-tert-butyl (2S)-2,5-dihydro-1H-pyrrole-2-carboxylate could be recovered by acidification of the aqueous layer and extraction with an organic solvent as described in the original reference.

### Step B: tert-Butyl (2R)-2-(hydroxymethyl)-2,5-dihydro-1H-pyrrole-1-carboxylate

The title compound was prepared from 1-tert-butyl 2-methyl (2R)-2,5-dihydro-1H-pyrrole-1,2-dicarboxylate (intermediate step A) according to the procedure for Example 1, Step A. ¹H (500 MHz, CDCl₃) δ 5.82 (dd, J=1.3, 4.6 Hz, 1H); 5.66-5.62 (m, 1H); 4.75 (br s, 1H); 4.20 (dd, J=1.9, 15.6 Hz, 1H); 4.10 (ddd, J=2.0, 5.5, 15.6 Hz, 1H); 3.80 (dd, J=2.1, 11.4 Hz, 1H); 3.58 (dd, J=7.5, 11.4 Hz, 1H), 1.52 (s, 9H). ppm.

### Step C: tert-Butyl (1R,2S,5S)-2-(hydroxymethyl)-6-oxa-3-azabicyclo[3.1.0]hexane-3-carboxylate

The title compound was prepared from tert-butyl (2R)-2-(hydroxymethyl)-2,5-dihydro-1H-pyrrole-1-carboxylate (intermediate step B) according to the procedure for Example 1, Step B. ¹H NMR (500 MHz, CDCl₃): δ 3.98 (d, J=10.8 Hz, 1H); 3.94-3.82 (m, 2H); 3.77 (br s, 1H); 3.74 (d, J=13 Hz, 1H); 3.62 (s, 1H); 3.42 (d, J=13 Hz, 1H); 1.46 (s, 9H). ¹³C (125 MHz, CDCl₃) 158.02, 64.19, 61.90, 58.58, 53.75, 49.71, 28.57 ppm. Also obtained was the minor more polar isomer 2 (anti isomer) tert-butyl (1S,2S,5R)-2-(hydroxymethyl)-6-oxa-3-azabicyclo[3.1.0]hexane-3-carboxylate. ¹H NMR (500 MHz, CDCl₃): δ 4.17 (t, J=5-2 Hz, 0.5H), 4.03 (t, J=4.2 Hz, 0.5H), 3.88 (d, J=13 Hz, 0.5H), 3.86-3.74 (m, 3H), 3.70-3.65 (m, 1H), 3.60 (d, J=3.0 Hz, 0.5H), 3.37 (dd, J=1.2, 13 Hz, 1H), 1-45 (br s, 9H). ¹³C (125 MHz, CDCl₃): δ 156.11 (154.96), 80.88 (80.62), 62.86 (62.37), 60.18 (60.07), 58.08 (57.29), 55.35 (54.89), 47.81 (47.75), 28.69 (28.62).

### Step D: tert-Butyl (1R,2S,5S)-2-({[tert-butyl(dimethyl)silyl]oxy}methyl)-6-oxa-3-azabicyclo[3.1.0]hexane-3-carboxylate

The title compound was prepared from tert-butyl (1R,2S,5S)-2-(hydroxymethyl)-6-oxa-3-azabicyclo[3.1.0]hexane-3-carboxylate (major isomer 1 of intermediate from Step C) according to the procedure for Example 1, step C. ¹H NMR (500 MHz, CDCl₃): δ 4.33 (br s, 0.5H); 4.15 (br s, 0.5H); 3.90 (t, J=2.5 Hz, 1H); 3.88-3.76 (m, 1H); 3.72 (br d, J=14.2 Hz, 1H); 3.65 (d, J=12.5 Hz, 1H); 3.60-3.52 (m, 1H); 3.46 (d, J=12.5 Hz, 1H); 1.46 (br s, 9H); 0.93 (s, 9H); 0.12 (s, 6H). ppm.

### Step E: tert-Butyl (2R,3S,4R)-2-({[tert-butyl(dimethyl)silyl]oxy}methyl)-3-(4-fluorophenyl)-4-hydroxypyrrolidine-1-carboxylate (more polar isomer) and tert-Butyl (2S,3S,4R)-2-({[tert-butyl(dimethyl)silyl]oxy}methyl)-4-(4-fluorophenyl)-3-hydroxypyrrolidine-1-carboxylate (less polar isomer)

The title compounds were prepared from 4-fluorophenylmagnesium bromide and tert-butyl (1R,2S,5S)-2-({[tert-butyl(dimethyl)silyl]oxy}methyl)-6-oxa-3-azabicycio[3.1.0]hexane-3-carboxylate (intermediate Step D) according to the procedure for Example 1, step D to provide two isomeric products. tert-Butyl (2R,3S,4R)-2-({[tert-butyl(dimethyl)silyl]oxy}methyl)-3-(4-fluorophenyl)-4-hydroxypyrrolidine-1-carboxylate, more polar isomer by TLC. More polar isomer by TLC: 130 mg (37%). ¹H NMR (500 MHz, CDCl₃): δ 6.90 (app t, J=8.2, 5 Hz, 2H), 6.77 (app. t, J=8.3 Hz, 2H), 4.44 (dd, J=2.4, 10.6 Hz, 1H), 4.24-4.16 (m, 1H); 4.04 (br s, 1H), 3.70-3.62 (m, 1H); 3.60 (d, J=10.6 Hz, 1H); 3.48 (dd, J=12.4, 15.6 Hz, 1H); 3.37 (s, 1H); 1.53 (s, 9H); 0.96 (s, 9H); 0.17 (s, 6H). MS: 426 (M+H), 326 (M+H-Boc). tert-Butyl (2S,3S,4R)-2-({[tert-butyl(dimethyl)silyl]oxy}methyl)-4-(4-fluorophenyl)-3-hydroxypyrrolidine-1-carboxylate, less polar isomer by TLC. ¹H NMR (500 MHz, CDCl₃): δ 7.26 dd, 2H, J=4.8, 8 Hz), 7.04 (app t, 2H); 4.44 (dd, J=8.45, 16 HZ, 1H); 4.24 (dd, J=4.0, 10.9 Hz, 0.5H), 4.10-4.02 (m, 2H); 3.97-3.92 (m, 0.5H); 3.82 (dd, J=8.5, 16.4 HZ, 1H), 3.52-3.40 (m, 2H), 3.32 (t, 10H, 10.5 Hz), 1.52 (s, 3.5H); 1.48 (s, 5.5H), 0.96 (s, 9H); 0.14 (s, 6H). MS: 426 (M+H), 326 (M+H-Boc)

### Step F: tert-Butyl (2R,3S,4R)-4-{[3,5-bis(trifluoromethyl)benzoyl]oxy}-2-({[tert-butyl(dimethyl)silyl]oxy}methyl)-3-(4-fluorophenyl)pyrrolidine-1-carboxylate

The title compound was prepared from tert-butyl (2R,3S,4R)-2-({[tert-butyl(dimethyl)silyl]oxy}methyl)-3-(4-fluorophenyl)-4-hydroxypyrrolidine-1-carboxylate (the more polar isomer of Step E) according to the procedure for Example 1, Step E. MS: 688 (M+Na); 566 (M+H-Boc).

### Step G: tert-butyl (2R,3S,4R)-4-({1-[3,5-bis(trifluoromethyl)phenyl]vinyl}oxy)-2-({[tert-butyl(dimethyl)silyl]oxy}methyl)-3-(4-fluorophenyl)pyrrolidine-1-carboxylate

To a 1 L 3-neck round bottom flask equipped with mechanical stirrer, thermocouple, addition funnel and N nitrogen line was added 40.8 g (61.3 mmol) of tert-butyl (2R,3S,4R)-4-{[3,5-bis(trifluoromethyl)benzoyl]oxy}-2-({[tert-butyl(dimethyl)silyl]oxy}methyl)-3-(4-fluorophenyl)pyrrolidine-1-carboxylate (more polar intermediate of Step F) in 400 mL dry THF. The solution was cooled to −5° C. and 150 mL (75 mmol) of a 0.5M solution of Tebbe reagent in toluene was added by addition funnel over 1.5 hr keeping the reaction mixture below 10° C. After completion of the addition, the reaction mixture was stirred at 0° C. for 2 hours. The reaction mixture was carefully quenched by carefully pouring into a stirred mixture of ice/water (˜2 L) in portion-wise fashion. EtOAc (1 L) was added and the mixture stirred at RT for ten minutes and then filtered through filter aid. The layers were separated and the aqueous layer back extracted with additional EtOAc. The organic layers were combined dried over anhydrous magnesium sulfate, filtered and the solvent removed under vacuum. The resulting orange oil and solids were dissolved in methylene chloride and flushed through a silica gel column eluting with methylene chloride to provide 37 g (91%) of the title compound as an orange oil. MS: 686 (M+Na); 607 (M+H-Boc).

### Step H: tert-Butyl (2R,3S,4R)-4-{(1R)-1-[3,5-bis(trifluoromethyl)phenyl]ethoxy}-2-({[tert-butyl(dimethyl)silyl]oxy}methyl)-3-(4-fluorophenyl)pyrrolidine-1-carboxylate

To a solution of 39 g (58.7 mmol) tert-butyl (2R,3S,4R)-4-({1-[3,5-bis(trifluoromethyl)phenyl]vinyl}oxy)-2-({[tert-butyl(dimethyl)silyl]oxy}methyl)-3-(4-fluorophenyl)pyrrolidine-1-carboxylate (intermediate step G) in 400 mL EtOH was added 2 g (0.05 by wt) 10% Pd—C. The resulting mixture was hydrogenated in two 500 mL pressure vessels at 40 PSI and RT for 2 hr. The reaction was filtered and the solvent of the filtrate removed under vacuum to give an approximately 4:1 mixture of undesired:desired product.

The undesired less polar isomer can be isomerized to a 1:1 mixture of less polar undesired and more polar desired isomer tert-butyl (2R,3S,4R)-4-{(1R)-1-[3,5-bis(trifluoromethyl)phenyl]ethoxy}-2-({[tert-butyl(dimethyl)silyl]oxy}methyl)-3-(4-fluorophenyl)pyrrolidine-1-carboxylate. After separation of the isomers by chromatography on silica gel and several recycles of the undesired less polar isomer the major desired was isolated in improved yield.

To 120 mL (120 mmol) of a 1.0M solution of potassium tert-butoxide in THF under nitrogen atmosphere, cooled to −78° C., was added a solution of ˜39 g of the above crude product in 400 mL dry THF dropwise over 30 min. The reaction mixture was slowly warmed in an ice/water bath then to RT. After the temperature reached RT, the mixture was heated at 40° C. for three hours. The reaction was cooled to RT, quenched with saturated aqueous ammonium chloride and extracted with excess EtOAc. The combined extracts were washed with water, dried over anhydrous sodium sulfate filtered and The residue was purified by column chromatography on silica gel eluting with EtOAc/Hexanes gradient (0/100 to 20/80 to provide 7.4 g (19%) tert-butyl (2R,3S,4R)-4-{(1R)-1-[3,5-bis(trifluoromethyl)phenyl]ethoxy}-2-({[tert-butyl(dimethyl)silyl]oxy}methyl)-3-(4-fluorophenyl)pyrrolidine-1-carboxylate, desired more polar diastereomer 2. ¹H NMR (500 MHz, CDCl₃): δ 7.73 (s, 1H); 7.52 (s, 2H); 7.02 (dd, J=6.2, 8.3 Hz, 2H); 7.00-6.94 (m, 2H); 4.45-4.34 (m, 1H); 4.12 (dd, J=6.8, 10.0 Hz, 1H); 4.04-3.95 (m, 1H); 3.92-3.80 (m, 2H); 3.82-3.78 (m, 1H); 3.70-3.62 (m, 1H); 3.56 (app. t, J=7.2, 1H); 3.50 (d, J=9 Hz, 1H); 3.24-3.30 (m, 1H); 1.52 (s, 3.5H); 1.48 (s, 4.5H); 1.42 (d, J=6.6 Hz, 3H), 0.94 (s, 9H); 0.06 (s, 3H); 0.04 (s, 3H)ppm. In addition, mixed fractions containing 4.6 g of >95% pure desired more polar was isolated and 16 g of a mixture containing a majority of undesired less polar diastereomer which could be further recycled to desired product by isomerization. Undesired less polar diastereomer ¹H NMR (500 MHz, CDCl₃): δ 7.80 (s, 1H); 7.58 (s, 2H); 7.24-7.16 (m, 2H); 7.04 (dd, J=6.2, 8.4 Hz, 21H); 4.50-4.40 (m, 1H); 4.08 (br. d, J=6.0 Hz, 1H); 3.92-3.83 (m, 2H); 3.80-3.76 (m, 1H); 3.72 (br. s, 1H); 3.68-3.55 (m, 2H); 3.26-3.20 (m, 1H); 3.15 (br. t, J=9.2 Hz, 1H); 1.46 (s, 3.5H); 1.44 (s, 4.5H); 1.36 (d, J=6.6 Hz, 3H), 0.94 (s, 9H); 0.07 (s, 3H); 0.05 (s, 3H)ppm.

### Step r: tert-butyl (2R,3S,4R)-4-{(1R)-1-[3,5-bis(trifluoromethyl)phenyl]ethoxy}-3-(4-fluorophenyl)-2-(hydroxymethyl)pyrrolidine-1-carboxylate

The title compound was prepared from tert-butyl (2R,3S,4R)-4-{(1R)-1-[3,5-bis(trifluoromethyl)phenyl]ethoxy}-2-({[tert-butyl(dimethyl)silyl]oxy}methyl)-3-(4-fluorophenyl)pyrrolidine-1-carboxylate (the more polar intermediate Step H) according to the procedure for Example 1, step H. ¹H NMR (500 MHz, CDCl₃): δ 7.72 (s, 1H); 7.46 (s, 2H); 7.06 (dd, J=5.2, 8.6 Hz, 2H); 6.97 (dd, J=8.7, 7.9 Hz, 2H); 4.45 (app q, J=6.3 Hz, 1H); 4.00 (dd, J=2.3, 11.0 Hz, 2H) 3.99 (overlapping br s, 1H); 3.72 (br s, 2H), 3.38 (dd, J=7.7, 10.6 Hz, 1H), 2.92 (br s, 1H); 153 (s, 9H); 1.41 (d, J=6.6 Hz, 3H)ppm. MS: 574 (M+Na), 451 (M+H-Boc).

### EXAMPLE 3

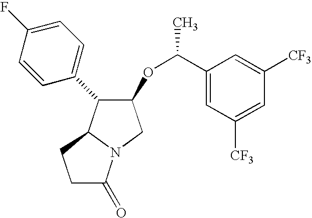

### (6R,7S,7aS)-6-{(1R)-1-[3,5-bis(trifluoromethyl)phenyl]ethoxy}-7-(4-fluorophenyl)hexahydro-3H-pyrrolizin-3-one

### Step A: tert-butyl (2R,3S,4R)-4-{(1R)-1-[3,5-bis(trifluoromethyl)phenyl]ethoxy}-3-(4-fluorophenyl)-2-formylpyrrolidine-1-carboxylate

To a stirred solution of 0.68 mL (1.36 mmol) oxalyl chloride in 15 mL dry methylene chloride under nitrogen atmosphere at −78° C. was added 0.19 mL (2.72 mmol) DMSO dropwise over 5 min by syringe. After ten min., to this mixture was added a solution of 375 mg (0.68 mmol) tert-butyl (2R,3S,4R)-4-{(1R)-1-[3,5-bis(trifluoromethyl)phenyl]ethoxy}-3-(4-fluorophenyl)-2-(hydroxymethyl)pyrrolidine-1-carboxylate (Example 2) in 1 mL dry methylene chloride. The reaction mixture was stirred at −78° C. for 1 hr, then 0.76 mL (5.44 mmol) TEA was added by syringe. The reaction mixture was stirred at −78° C. for 15 min then warmed to room temperature and stirred an additional hr. The reaction mixture was quenched with aq. 1N HCL (˜15 mL) and transferred to a separatory funnel. The reaction mixture was extracted with EtOAc (2×15 mL). The combined organic extracts were washed with water (15 mL) then brine (15 mL), dried over anhydrous sodium sulfate, filtered and evaporated under vacuum afford the title compound. The resulting crude product was purified by preparative TLC plate eluding with ethyl acetate/hexane (1/4) to afford 350 mg (95%) of the title compound.

### Step B: tert-butyl (2S,3S,4R)-4-{(1R)-1-[3,5-bis(trifluoromethyl)phenyl]ethoxy}-3-(4-fluorophenyl)-2-[(1E)-3-t-butoxy-3-oxoprop-1-en-1-yl]pyrrolidine-1-carboxylate

To a solution of 977 mg (1.78 mmol) tert-butyl (2R,3S,4R)-4-{(1R)-1-[3,5-bis(trifluoromethyl)phenyl]ethoxy}-3-(4-fluorophenyl)-2-formylpyrrolidine-1-carboxylate (step A) in 30 mL dry methylene chloride under nitrogen atmosphere was added 670 mg (1.78 mmol) (t-butoxycarbonylmethylene) triphenylphosphorane. The resulting mixture was stirred at RT for 16 hr. The solvent was removed under vacuum and the residue purified by Horizon MPLC using a gradient eluting system of 0-20% ethyl acetate in hexane to afford 1.01 g (88%) of the title compound. MS: 648.2 (MH+)

### Step C: tert-butyl (2S,3S,4R)-4-{(1R)-1-[3,5-bis(trifluoromethyl)phenyl]ethoxy}-3-(4-fluorophenyl)-2-(3-t-butoxy-3-oxopropyl)pyrrolidine-1-carboxylate

To a solution of 1.01 g (1.56 mmol) tert-butyl (2S,3S,4R)-4-{(1R)-1-[3,5-bis(trifluoromethyl)phenyl]ethoxy}-3-(4-fluorophenyl)-2-[(1E)-3-t-butoxy-3-oxoprop-1-en-1-yl]pyrrolidine-1-carboxylate (step B) in 30 mL ethanol under nitrogen atmosphere was added 100 mg 10% Pd—C catalyst. The resulting mixture was stirred under hydrogen balloon atmosphere at RT. After several hours, the catalyst was filtered through filter-aid and the solvent was removed under vacuum to afford 945 mg (94%) of the title compound. MS: 650 (MH)⁺, 594 (M-56; M-t-bu)⁺, 550 (M-100; M-BOC)⁺.

### Step D: 3-[(2S,3S,4R)-4-{(1R)-1-[3,5-Bis(trifluoromethyl)phenyl]ethoxy}-3-(4-fluorophenyl)pyrrolidin-2-yl]propanoic acid hydrochloride

The title compound was prepared by treatment of tert-butyl (2S,3S,4R)-4-{(1R)-1-[3,5-bis(trifluoromethyl)phenyl]ethoxy}-3-(4-fluorophenyl)-2-(3-t-butoxy-3-oxopropyl)pyrrolidine-1-carboxylate (intermediate step C) with 30 mL of 4N HCl in dioxane for one hour at room temperature. Concentration in vacuo afforded 770 mg of the title compound; no further purification was necessary. ¹H NMR (500 MHz, CD₃OD): δ 7.78 (s, 1H); 7.64 (s, 2H); 7.21 (dd, J=5.0, 8.7 Hz, 2H); 7.20 (app. t, J=8.7 Hz, 2H); 4.72 (app q, J=6.2 Hz, 1H); 4.22 (ddd, J=2.7, 7.3, 12.0 Hz, 1H) 3.82 (dd, J=7.3, 12.2 Hz, 1H); 3.66 (ddd, J=3.2, 4.6, 12.0 Hz, 1H), 3.50 (dd, J=4.6, 12.3 Hz, 1H), 3.14 (dd, J=7.4, 12.0 Hz, 1H), 2.35 (app. dd, J=6.8, 12.7 Hz, 2H),); 2.06-1.96 (m, 1H); 1.98-1.87 (m, 1H); 1.43 (d, J=6.7 Hz, 3H)ppm. MS: 494 (MH)⁺; 516 (M+Na)⁺.

### Step E: (6R,7S,7aS)-6-{(1R)-1-[3,5-bis(trifluoromethyl)phenyl]ethoxy}-7-(4-fluorophenyl)hexahydro-3H-pyrrolizin-3-one

To a solution of 768 mg (1.45 mmol) 3-[(2S,3S,4R)-4-{(1R)-1-[3,5-Bis(trifluoromethyl)phenyl]ethoxy}-3-(4-fluorophenyl)pyrrolidin-2-yl]propanoic acid hydrochloride (intermediate step D), 20 mg (0.15 mmol) DMAP, and 253 μL (1.45 mmol) DIEA in 75 mL dichloromethane under nitrogen atmosphere was added 556 mg (2.90 mmol) EDC and the resulting solution stirred overnight at room temperature. The reaction mixture was washed with 1N HCl solution (20 mL), followed by saturated NaHCO₃ solution (20 mL) and then brine (30 mL). The organic solution was dried over sodium sulfate, filtered through a fritted funnel, and concentrated in vacuo. The residue was purified by eight preparative TLC plates eluting with ethyl acetate to afford 600 mg (88%) of the title compound. ¹H NMR (500 MHz, CDCl₃): δ 7.78 (s, 1H), 7.46 (s, 2H), 7.08-7.04 (m, 2H), 7.00 (app t, J=8.7 Hz, 2H), 4.44 (q, J=6.6 Hz, 1H), 4.38 (q, J=6.4 Hz, 1H), 4.13 (q, J=7.1H, 1H), 3.80-3.73 (m, 2H), 3.66-3.62 (m, 1H), 3.05-2.90 (m, 2H), 2.80-2.74 (m, 1H), 1.92-1.86 (m, 1H), 1.42 (d, J=6.4 Hz, 31H). MS: 476 (MH)⁺; 498 (M+Na)⁺.

### EXAMPLE 4

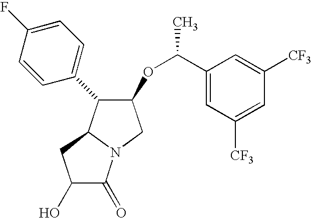

### (6R,7S,7aS)-6-{(1R)-1-[3,5-bis(trifluoromethyl)phenyl]ethoxy}-7-(4-fluorophenyl)-2(R or S)hydroxyhexahydro-3H-pyrrolizin-3-one

### Step A: ((6R,7S,7aS)-6-{(1R)-1-[3,5-bis(trifluoromethyl)phenyl]ethoxy}-7-(4 or S)hydroxyhexahydro-3H-pyrrolizin-3-one

To a solution of 100 mg (0.21 mmol) (6R,7S)-6-{(1R)-1-[3,5-bis(trifluoromethyl)phenyl]ethoxy}-7-(4-fluorophenyl)hexahydro-3H-pyrrolizin-3-one in 10 mL anhydrous THF under nitrogen atmosphere cooled to −78° C. via dry ice/acetone bath was added 250 μL of 1.0M solution of LHMDS in THF. The resulting mixture was stirred at −78° C. for ten minutes, and then allowed to warm to −20° C. over one hour. MoOPh (190 mg, 0.42 mmol) was then added as a solid to the solution and the resulting mixture was stirred in the dark by wrapping the round bottom flask with aluminum foil. The solution was stirred for one hour allowing to warm to room temperature. The mixture was partitioned with ethyl acetate and 2N HCl and the organic layer was then further washed with brine (10 mL), dried over magnesium sulfate, filtered through a fritted funnel, and concentrated in vacuo. The residue was purified by preparative TLC plate eluting with methanol/ethyl acetate (1/9) to afforded two separate isomers of the title compound. Isomer 1 was named for the less polar isomer (38.6 mg), while isomer 2 was the more polar isomer (26.8 mg). Isomer 1: ¹H NMR (50 MHz, CDCl₃): δ 7.72 (s, 1H), 7.44 (s, 2H), 7.10-7.04 (m, 2H), 6.99 (app t, J=8.7 Hz, 2H), 4.51-4.44 (m, 2H), 4.18 (app q, J=6.6 Hz, 1H), 4.06 (dt, J=6.3, 11.5 Hz, 1H), 3.68 (d, J=6.4 Hz, 2H), 3.44 (br s, 1H), 3.14 (q, J=7.4 Hz, 1H), 2.75 (dd, J=8.5, 10.3 Hz, 1H), 2.24-2.15 (m, 1H), 2.13-2.07 (m, 1H), 1.41 (d, J=6.4 Hz, 3H). MS: 492 (MH)⁺; 514 (M+Na)⁺. Isomer 2: 1-NMR (500 MHz, CDCl₃): δ 7.74 (s, 1H), 7.46 (s, 2H), 7.08-7.04 (m, 2H), 7.00 (app t, J=8.7 Hz, 2H), 4.58 (t, J=8.8 Hz, 1H), 4.48 (q, J=6.4 Hz, 1H), 4.18 (app q, J=6.7 Hz, 1H), 3.78-3.72 (m, 2H), 3.64 (dd, J=7.3, 11.9 Hz, 1H), 3.02 (br s, 1H), 2.94 (t, J=8.8 Hz, 1H), 2.74-2.67 (m, 1H), 1.90-1.84 (m, 1H), 1.43 (d, J=6.4 Hz, 3H). MS: 492 (MH)⁺; 514 (M+Na)⁺.

### EXAMPLE 5

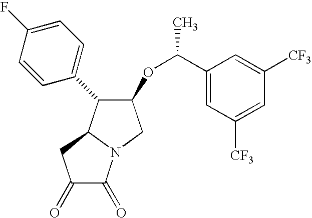

### (6R,7S,7aS)-6-{(1R)-1-[3,5-bis(trifluoromethyl)phenyl]ethoxy}-7-(4-fluorophenyl)tetrahydro-1H-pyrrolizine-2,3-dione

### Step A: (6R,7S,7aS)-6-{(1R)-1-[3,5-bis(trifluoromethyl)phenyl]ethoxy}-7-(4-fluorophenyl)tetrahydro-1H-pyrrolizine-2,3-dione

To a solution of 140 mg (2.85 mmol) (6R,7S)-6-{(1R)-1-[3,5-bis(trifluoromethyl)phenyl]ethoxy}-7-(4-fluorophenyl)-2(R or S)hydroxyhexahydro-3H-pyrrolizin-3-one in 10 mL anhydrous dichloromethane under nitrogen atmosphere was added via syringe 2.02 mL (5.70 mmol) of a 15% Dess-Martin solution in dichloromethane and the resulting solution was stirred for 2 hours at room temperature. The reaction mixture was applied directly to a preparative TLC plate eluding with ethyl acetate/hexane (3/2) to afford 44 mg (32%) of the title compound. MS: 490 (MH)⁺; 512 (M+Na)⁺.

### EXAMPLE 6

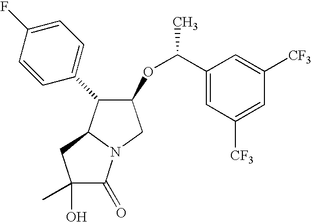

### (6R,7S,7aS)-6-{(1R)-1-[3,5-bis(trifluoromethyl)phenyl]ethoxy}-7-(4-fluorophenyl)-2-hydroxy-2-methylhexahydro-3H-pyrrolizin-3-one

### Step A: (6R,7S,7aS)-6-{(1R)-1-[3,5-bis(trifluoromethyl)phenyl]ethoxy}-7-(4-fluorophenyl)-2-hydroxy-2-methylhexahydro-3H-pyrrolizin-3-one

To a solution of 40 mg (0.09 mmol) (6R,7S)-6-{(1R)-1-[3,5-bis(trifluoromethyl)phenyl]ethoxy}-7-(4-fluorophenyl)tetrahydro-1H-pyrrolizine-2,3-dione in 1 mL anhydrous toluene under nitrogen atmosphere was added via syringe 320 μL (0.45 mmol) of 1.4 M methylmagnesium bromide in THF and the resulting solution was stirred for 3 hours at room temperature. The reaction mixture was quenched with saturated ammonium chloride solution (1 mL) and the organic layer separated. The aqueous layer was washed with ethyl acetate (2×5 mL). The organics were then combined, dried over sodium sulfate, filtered through a fritted funnel, and concentrated in vacuo. The residue was purified by preparative TLC plate eluding with ethyl acetate to afford 18 mg (42%) of the title compound as a single isomer. MS: 506 (MH)⁺; 528 (M+Na)⁺.

### EXAMPLE 7

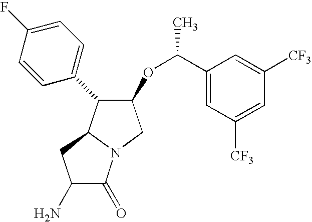

### Hydrochloride

### (6R,7S,7aS)-2-amino-6-{(1R)-1-[3,5-bis(trifluoromethyl)phenyl]ethoxy}-7-(4-fluorophenyl)hexahydro-3H-pyrrolizin-3-one

### Step A: (6R,7S,7aS)-6-{(1R)-1-[3,5-bis(trifluoromethyl)phenyl]ethoxy}-7-(4-fluorophenyl)-3-oxohexahydro-1H-pyrrolizin-2-yl methanesulfonate

To a solution of 50 mg (0.10 mmol) (6R,7S)-6-{(1R)-1-[3,5-bis(trifluoromethyl)phenyl]ethoxy}-7-(4-fluorophenyl)-2(R or S)-hydroxyhexahydro-3H-pyrrolizin-3-one in 3 mL anhydrous dichloromethane under nitrogen atmosphere cooled to 0° C. was added 10 μL mesyl chloride followed by 18 μL TEA The resulting solution was stirred overnight allowing to warn to room temperature. The mixture was concentrated in vacuo and the residue purified by preparative TLC plate eluting with ethyl acetate to afford 47 mg (75%) of the title compound.

### Step B: (6R,7S,7aS)-2-azido-6-{(1R)-1-[3,5-bis(trifluoromethyl)phenyl]ethoxy}-7-(4-fluorophenyl)hexahydro-3H-pyrrolizin-3-one

To a solution of 47 mg (0.07 mmol) (intermediate from step A) in 2 mL anhydrous DMF was added 48 mg (0.75 mmol) sodium azide and the resulting mixture was set under nitrogen atmosphere and refluxed overnight. The solution was poured into 5 mL water and extracted with ether (3×5 mL). The ether layers were combined and washed with water (5 mL) and then brine (5 mL). The organics were combined and treated as stated in Example 3, step E. The residue was purified by preparative TLC plate eluting with ethyl acetate/hexane (3/2) to afford 36 mg (93%) of the title compound. MS: 517 (MH)⁺.

### Step C: (6R,7S,7aS)-2-amino-6-{(1R)-1-[3,5-bis(trifluoromethyl)phenyl]ethoxy}-7-(4-fluorophenyl)hexahydro-3H-pyrrolizin-3-one

A solution of 34 mg (0.06 mmol) (intermediate from step B) in 1 mL ethanol and 117 μL of 4N HCl in dioxane was treated with 10% palladium on carbon (5 mg) and the resulting suspension set under hydrogen atmosphere and stirred for 3 hours at room temperature. The catalyst was filtered off using a Gilmen 0.45 μM PTFE syringe filter and the filtrate was concentrated in vacuo. The residue was purified by preparative TLC plate eluting with 1% NH₄OH/10% methanol/89% dichloromethane to afford the title compound as a free base. The free base was converted to the HCl salt by dissolving the compound in dichloromethane and treating the solution with 4N HCl in dioxane (2 equivalent). The solution was then concentrated in vacuo and stored on the high vacuum pump for 2 hours to afford 32 mg (98%) of the title compound. MS: 491 (MH)⁺

### EXAMPLE 8

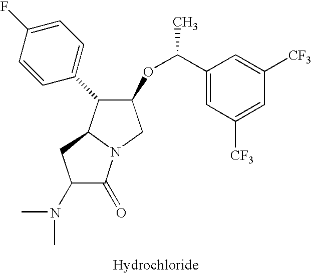

### (6R,7S,7aS)-6-{(1R)-1-[3,5-bis(trifluoromethyl)phenyl]ethoxy}-2-(dimethylamino)-7-(4-fluorophenyl)hexahydro-3H-pyrrolizin-3-one

### Step A: (6R,7S,7aS)-6-{(1R)-1-[3,5-bis(trifluoromethyl)phenyl]ethoxy}-2-(dimethylamino)-7-(4-fluorophenyl)hexahydro-3H-pyrrolizin-3-one

To a solution of 22 mg (0.04 mmol) (6R,7S,7aS)-2-amino-6-{(1R)-1-[3,5-bis(trifluoromethyl)phenyl]ethoxy}-7-(4-fluorophenyl)hexahydro-3H-pyrrolizin-3-one in 1 mL anhydrous dichloromethane and 8 μL (0.04 mmol) DIEA under nitrogen atmosphere was added crushed 4A molecular sieves followed by 0.1 mL Formalin. After 5 minutes of stirring, the solution was then treated with Na(OAc)₃BH and the resulting suspension stirred vigorously at room temperature, overnight. The mixture was quenched with saturated sodium bicarbonate solution and extracted with dichloromethane (2×5 mL). The organics were combined and treated as stated in Example 3, step E. The residue was purified by preparative TLC plate eluding with methanol/dichloromethane (1/9) to afford the title compound as its free base. Conversion to the HCl salt was accomplished according to the procedure for Example GJM-5, step C. MS: 519 (MH)⁺.

### EXAMPLE 9 and 10

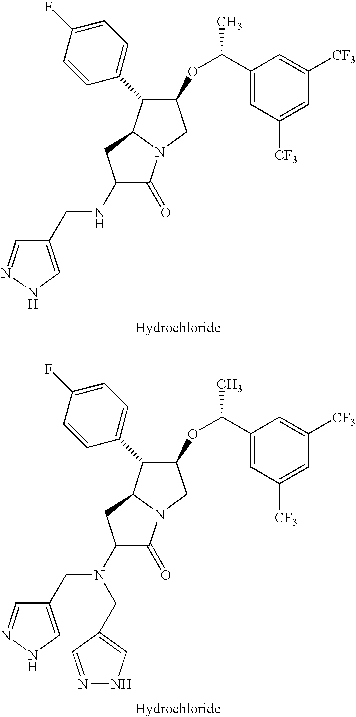

### (6R,7S,7aS)-6-{(1R)-1-[3,5-bis(trifluoromethyl)phenyl]ethoxy}-7-(4-fluorophenyl)-2-[(1H-pyrazol-4-ylmethyl)amino]hexahydro-3H-pyrrolizin-3-one and (6R,7S,7aS)-2-[bis(1H-pyrazol-4-ylmethyl)amino]-6-{(1R)-1-[3,5-bis(trifluoromethyl)phenyl]ethoxy}-7-(4-fluorophenyl)hexahydro-3H-pyrrolizin-3-one

### Step A: (6R,7S,7aS)-6-{(1R)-1-[3,5-bis(trifluoromethyl)phenyl]ethoxy}-7-(4-fluorophenyl)-2-[(1H-pyrazol-4-ylmethyl)amino]hexahydro-3H-pyrrolizin-3-one and (6R,7S,7aS)-2-[bis(1H-pyrazol-4-ylmethyl)amino]-6-{(1R)-1-[3,5-bis(trifluoromethyl)phenyl]ethoxy}-7-(4-fluorophenyl)hexahydro-3H-pyrrolizin-3-one

To a solution of 20 mg (0.04 mmol) (6R,7S,7aS)-2-amino-6-{(1R)-1-[3,5-bis(trifluoromethyl)phenyl]ethoxy}-7-(4-fluorophenyl)hexahydro-3H-pyrrolizin-3-one in 1 mL anhydrous dichloromethane and 7 μL (0.04 mmol) DIEA under nitrogen-atmosphere was added crushed 4A molecular sieves followed by 40 mg (0.04 mmol) aromatic heterocyclic aldehyde. After 5 minutes of stirring, the solution was then treated with 40 mg (0.2 mmol) Na(OAc)₃BH and the resulting suspension stirred vigorously at room temperature, overnight. The mixture was quenched with saturated sodium bicarbonate solution and extracted with dichloromethane (2×5 mL). The organics were combined and treated as stated in Example 3, step E. The residue was purified by preparative TLC plate eluding with methanol/dichloromethane (1/9) to afford two products. The less polar product was found to be the mono substituted compound 5.3 mg (22%), MS: 571 (MH)⁺, and the slightly more polar product 11.2 mg (48%) was di-substituted, MS: 651 (MH)⁺. Conversion to the HCl salts of each were accomplished according to the procedure for Example GJM-5, step C.

Using the procedures similar to those described above the following Examples were prepared.

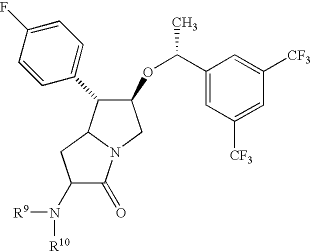

| | | | parent ion |
|---|---|---|---|
| Example | R⁹ | R¹⁰ | (MH)⁺ |
| Example 11 | 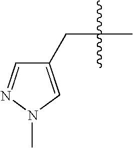 | H | 585 |
| Example 12 | 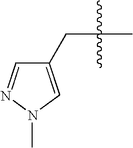 | 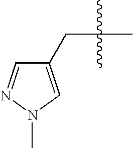 | 679 |
| Example 13 | 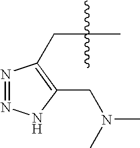 | H | 629 |

### EXAMPLE 14

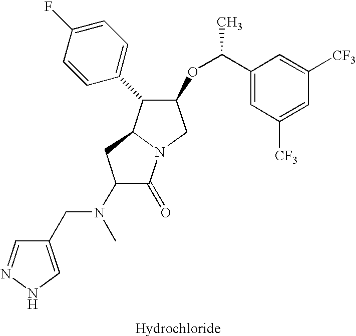

### (6R,7S,7aS)-6-{(1R)-1-[3,5-bis(trifluoromethyl)phenyl]ethoxy}-7-(4-fluorophenyl)-2-[methyl(1H-pyrazol-4-ylmethyl)amino]hexahydro-3H-pyrrolizin-3-one

### Step A: (6R,7S,7aS)-6-{(1R)-1-[3,5-bis(trifluoromethyl)phenyl]ethoxy}-7-(4-fluorophenyl)-2-[methyl(H-pyrazol-4-ylmethyl)amino]hexahydro-3H-pyrrolizin-3-one

The title compound was prepared from (6R,7S,7aS)-6-{(1R)-1-[3,5-bis(trifluoromethyl)phenyl]ethoxy}-7-(4-fluorophenyl)-2-[(1H-pyrazol-4-ylmethyl)amino]hexahydro-3H-pyrrolizin-3-one (less polar product of step A) according to the procedure for Example 8. MS: 585 (MH)⁺.

### EXAMPLE 15

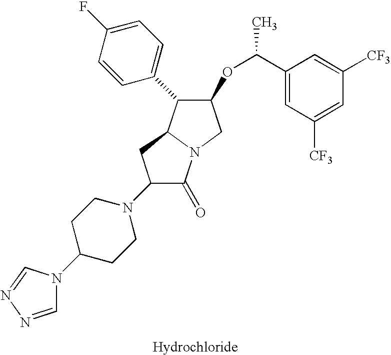

### (6R,7S,7aS)-6-{(1R)-1-[3,5-bis(trifluoromethyl)phenyl]ethoxy}-7-(4-fluorophenyl)-2-[4-(4H-1,2,4-triazol-4-yl)piperidin-1-yl]hexahydro-3H-pyrrolizin-3-one

### Step A: (6R,7S,7aS)-6-{(1R)-1-[3,5-bis(trifluoromethyl)phenyl]ethoxy}-7-(4-fluorophenyl)-2-[4-(4H-1,2,4-triazol-4-yl)piperidin-1-yl]hexahydro-3H-pyrrolizin-3-one

To a solution of 44 mg (0.09 mmol) (6R,7S,7aS)-6-{(1R)-1-[3,5-bis(trifluoromethyl)phenyl]ethoxy}-7-(4-fluorophenyl)tetrahydro-1H-pyrrolizine-2,3-dione in 0.5 mL methanol was added 28 mg (0.18 mmol) 4-(1,3,4-triazole)-piperidine and the resulting solution heated to 60° C. and stirred overnight. The reaction mixture was cooled to room temperature and 10 mg 10% palladium on carbon was added to the solution. The resulting suspension was set under hydrogen atmosphere and stirred at room temperature for 3 hours. The hydrogen was removed and the catalyst was filtered off using a Gilmen 0.45 μM PTFE syringe filter disc. The filtrate was concentrated in vacuo and the residue purified by preparative TLC plate eluding with 1% ammonium hydroxide/10% methanol/89% dichloromethane to afford 4.45 mg (10%) of the title compound as its free base. Conversion to the HCl salt was accomplished according to the procedure for Example 7, step C. MS: 626 (MH)⁺.

Using the procedures similar to those described above the following Examples were prepared.

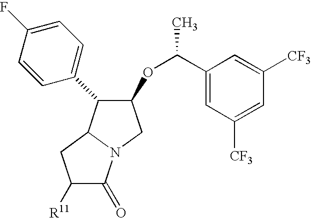

| Example | R¹¹ | parent ion |
|---|---|---|
| Example 16 | 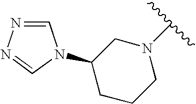 | 626 |
| Example 17 | 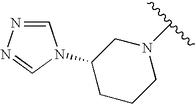 | 626 |
| Example 18 | 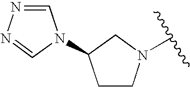 | 612 |
| Example 19 | 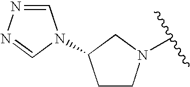 | 612 |
| Example 20 | 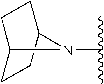 | 572 |
| Example 21 | 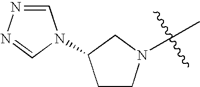 | 598 |

### EXAMPLE 22

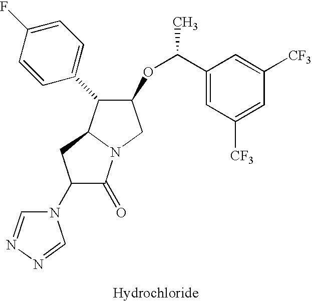

### (6R,7S,7aS)-6-{(1R)-1-[3,5-bis(trifluoromethyl)phenyl]ethoxy}-7-(4-fluorophenyl)-2-(4H-1,2,4-triazol-4-yl)hexahydro-3H-pyrrolizin-3-one

### Step A: 6R,7S,7 as)-6-{(1R)-1-[3,5-bis(trifluoromethyl)phenyl]ethoxy}-7-(4-fluorophenyl-2-(4H-1,2,4-triazol-4-yl)hexahydro-3H-pyrrolizin-3-one

To a solution of 20 mg (0.04 mmol) (6R,7S,7aS)-2-amino-6-{(1R)-1-[3,5-bis(trifluoromethyl)phenyl]ethoxy}-7-(4-fluorophenyl)hexahydro-3H-pyrrolizin-3-one in 1 mL of anhydrous toluene was added 11 mg (0.08 mmol) N,N-dimethylforamide azide followed by a catalytic amount of p-toluenesulfonic acid. The resulting solution was heated to reflux and stirred overnight. The solution was cooled to room temperature and concentrated in vacuo. The residue was purified by preparative TLC plate eluting with 82/6/6/6 ethyl acetate/methanol/acetonitrile/water to give the title compound as two separate isomers. The less polar isomer (10 mg, 45%) was labeled Isomer 1 while the more polar isomer (8 mg, 36%) was labeled Isomer 2. Conversion to the HCl salts of each were accomplished according to the procedure for Example 7, step C. ¹H NMR (500 MHz, CDCl₃): Isomer 1: δ 8.32 (s, 2H), 7.72 (s, 1H), 7.48 (s, 2H), 7.11-7.07 (m, 2H), 7.02 (app t, J=8.7 Hz, 2H), 5.20 (dd, J=8.0 Hz, 1H), 4.52 (q, J=6.5 Hz, 1H), 4.10-4.04 (m, 1H), 3.90 (dd, J=6.0, 12.3 Hz, 1H), 3.70-3.64 (m, 1H), 3.12-3.00 (m, 2H), 1.90-1.84 (m, 1H), 1.41 (d, J=6.4 Hz, 3H). MS: 543 (MH)⁺; 565 (M+Na)⁺. Isomer 2: δ 8.36 (s, 2H), 7.76 (s, 1H), 7.51 (s, 2H), 7.11-7.07 (m, 2H), 7.02 (app t, J=8.7 Hz, 2H), 5.20 (dd, J=8.0 Hz, 1H), 4.52 (q, J=6.5 Hz, 1H), 4.02 (td, J=6.0, 8.6 Hz, 1H), 3.90 (dd, J=6.0, 12.3 Hz, 1H), 3.67 (dd, J=6.4, 12.3 Hz, 1H), 3.12-3.00 (m, 2H), 2.23 (td, J=8.6, 12.0 Hz, 1H), 1.45 (d, J=6.4 Hz, 3H). MS: 543 (MH)⁺; 565 (M+Na)⁺.

### EXAMPLE 23

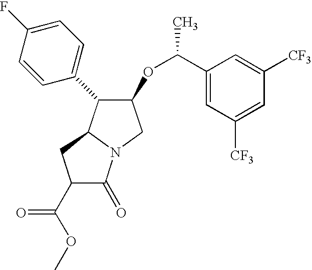

### methyl (6R,7S,7aS)-6-{(1R)-1-[3,5-bis(trifluoromethyl)phenyl]ethoxy}-7-(4-fluorophenyl)-3-oxohexahydro-1H-pyrrolizine-2-carboxylate

### Step A: methyl (6R,7S,7aS)-6-{(1R)-1-[3,5-bis(trifluoromethyl)phenyl]ethoxy}-7-(4-fluorophenyl)-3-oxohexahydro-1H-pyrrolizine-2-carboxylate

To a solution of 2.40 g (5.04 mmol) (6R,7S,7aS)-6-{(1R)-1-[3,5-bis(trifluoromethyl)phenyl]ethoxy}-7-(4-fluorophenyl)hexahydro-3H-pyrrolizin-3-one and 431 μL (5.12 mmol) dimethylcarbonate in 15 mL anhydrous THF under nitrogen atmosphere cooled to −78° C. by dry ice/acetone bath was added dropwise via syringe 5.05 mL of 2.0 M LDA in heptane/THF/ethylbenzene. The resulting solution was stirred for one hour at −78° C. before quenching with saturated solution of ammonium chloride. The mixture was extracted with ether (2×50 mL) and the organics were then combined, dried over magnesium sulfate, filtered through a fritted funnel, and the filtrate concentrated in vacuo. The residue was purified by use of the Horizon MPLC eluting with a gradient eluant of 50-100% ethyl acetate in hexanes to afford 2.26 g (84%) of the title compound as a 1:1 mixture of diastereomers. MS: 534 (MH)⁺; 556 (M+Na)⁺.

### EXAMPLE 24

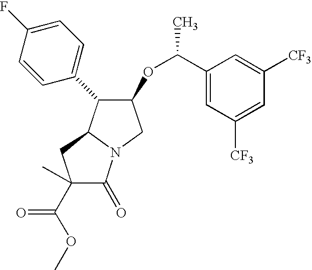

### methyl (6R,7S,7aS)-6-{(1R)-1-[3,5-bis(trifluoromethyl)phenyl]ethoxy}-7-(4-fluorophenyl)-2-methyl-3-oxohexahydro-1H-pyrrolizine-2-carboxylate

### Step A: methyl (6R,7S,7aS)-6-{(1R)-1-[3,5-bis(trifluoromethyl)phenyl]ethoxy}-7-(4-fluorophenyl)-2-methyl-3-oxohexahydro-1H-pyrrolizine-2-carboxylate

To a solution of 300 mg (0.61 mmol) methyl (6R,7S,7aS)-6-{(1R)-1-[3,5-bis(trifluoromethyl)phenyl]ethoxy}-7-(4-fluorophenyl)-3-oxohexahydro-1H-pyrrolizine-2-carboxylate in 3 mL anhydrous THF under nitrogen atmosphere, cooled to −78° C. by dry ice/acetone bath was added dropwise via syringe 765 μL of 1.0 M LHMDS in THF and the resulting solution stirred for one hour allowing to warm to −20° C. Once the temperature was at −20° C., iodomethane (69 μL, 1.11 mmol) was introduced dropwise via syringe and the solution was stirred an additional hour, allowing to warm to room temperature. The reaction was quenched with saturated solution of ammonium chloride and then extracted with ether (2×10 mL). The organics were combined and treated as stated in Example 3, step E. The residue was purified by preparative TLC plate eluting with ethyl acetate/hexane (4/1) to afford 264 mg (88%) of the title compound as a 10:1 mixture of diastereomers. MS: 548 (MH)⁺; 570 (M+Na)⁺.

### EXAMPLE 25 and 26

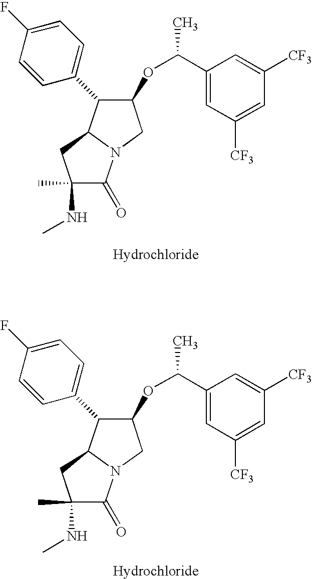

### (2(R or S)-6R,7S,7aS)-6-{(1R)-1-[3,5-bis(trifluoromethyl)phenyl]ethoxy}-7-(4-fluorophenyl)-2-methyl-(2-methylamino)hexahydro-3H-pyrrolizin-3-one

### Step A: (6R,7S,7aS)-6-{(1R)-1-[3,5-bis(trifluoromethyl)phenyl]ethoxy}-7-(4-fluorophenyl)-2-methyl-3-oxohexahydro-1H-pyrrolizine-2-carboxylic acid

To a solution of 100 mg (0.18 mmol) methyl (6R,7S,7aS)-6-{(1R)-1-[3,5-bis(trifluoromethyl)phenyl]ethoxy}-7-(4-fluorophenyl)-2-methyl-3-oxohexahydro-1H-pyrrolizine-2-carboxylate in 3 mL THF/water/methanol (1:1:1) was added 11.4 mg (0.91 mmol) lithium hydroxide and the resulting mixture heated to 60° C. and stirred overnight. The solution was cooled to room temperature and concentrated in vacuo to evaporate all organic solvent. The aqueous was then acidified to pH=3 by addition of 2N HCl and then extracted with ethyl acetate (3×5 mL). The organics were combined and treated as stated in Example 3, step E. The residue was purified by Gilson reverse phase prep-HPLC eluting with a gradient eluant of 25-75% acetonitrile in water buffered with 0.1% TFA to afford 98 mg (99%) of the title compound as a ˜8:1 mixture of diastereomers determined by ¹HNMR analysis MS: 534 (MH)⁺; 556 (M+Na)⁺, 516 (M-OH)⁺.

### Step B: benzyl[(2(R or S)-6R,7S,7aS)-6-{(1R)-1-[3,5-bis(trifluoromethyl)phenyl]ethoxy}-7-(4-fluorophenyl)-2-methyl-3-oxohexahydro-1H-pyrrolizin-2-yl]carbamate

To a solution of 98 mg (0.18 mmol) (intermediate step A) in 14 mL anhydrous toluene was added 50 μL DPPA and 32 μL TEA. The resulting solution was refluxed for one hour; then, cooled to room temperature. To the mixture was added 100 μL benzyl alcohol and the resulting solution stirred at room temperature overnight. The mixture was concentrated in vacuo and the residue purified by preparative TLC plate eluting with ethyl acetate/hexane (4/1) to afford two single diastereomers of the title compound. The less polar diastereomer (42 mg, 42%) was labeled Isomer 1, while the more polar diastereomer (19 mg, 19%) was labeled Isomer 2. Isomer 1 and 2 MS: 595 (MH)⁺.

### Step C: Benzyl[(2(R or S)-6R,7S,7aS)-6-{(1R)-1-[3,5-bis(trifluoromethyl)phenyl]ethoxy}-7-(4-fluorophenyl)-2-methyl-3-oxohexahydro-1H-pyrrolizin-2-yl]methylcarbamate

To a solution of 8 mg (0.01 mmol) Isomer 1, step B in 1 mL anhydrous DMF cooled to 0° C., under nitrogen atmosphere was added 1.3 mg (0.02 mmol) NaH and the resulting solution stirred for 15 minutes. Iodomethane (1 μL, 0.02 mmol) was the added and the resulting solution stirred for two hours allowing to warm to room temperature. The reaction was quenched with saturated solution of ammonium chloride and extracted with ether (3×3 mL). The organics were combined and treated as stated in Example 3, step E. The residue was purified by preparative TLC plate eluting with ethyl acetate/hexane (4/1) to afford the title compound 6.2 mg (74%). Isomer 2 was treated with the same procedure to afford 5.8 mg (68%) of the diastereomer. Isomer 1 and 2 MS: 609 (MH)⁺.

### Step D: (2(R or S)-6R,7S,7aS)-6-{(1R)-1-[3,5-bis(trifluoromethyl)phenyl]ethoxy}-7-(4-fluorophenyl)-2-methyl-(2-methylamino)hexahydro-3H-pyrrolizin-3-one

To a solution of 4 mg (0.006 mmol) Isomer 1, step C in 0.5 mL ethanol and 1.5 μL 4N HCl in dioxane was added 2 mg 10% palladium on carbon. The resulting suspension set under hydrogen atmosphere and stirred at room temperature for 2 hours. The catalyst was filtered off by using a Gilmen 0.45 μM PTFE syringe filter disc and the filtrate concentrated in vacuo. The residue was purified by preparative TLC plate eluting with 1% NH₄OH/10% methanol/89% dichloromethane to afford 2.86 mg (91%) of the less polar Isomer 1 titled compound. Isomer 2 was also treated with the same procedure to afford 1.97 mg (63%) of the more polar diastereomer. Conversion to the HCl salts of each were accomplished according to the procedure for Example 7, step C. Isomer 1 and 2: MS: 519 (MH)⁺.

### EXAMPLE 27 and 28

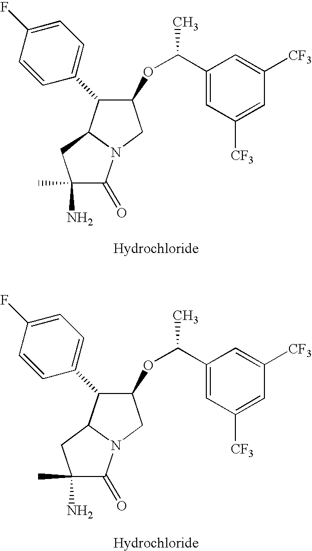

### (2(R or S)-6R,7S,7 as)-2-amino-6-{(1R)-1-[3,5-bis(trifluoromethyl)phenyl]ethoxy}-7-(4-fluorophenyl)-2-methylhexahydro-3H-pyrrolizin-3-one

### Step A: (2(R or S)-6R,7S,7aS)-2-amino-6-{(1R)-1-[3,5-bis(trifluoromethyl)phenyl]ethoxy}-7-(4-fluorophenyl)-2-methylhexahydro-3H-pyrrolizin-3-one

The title compounds were prepared from benzyl[(2(R or S)-6R,7S,7aS)-6-{(1R)-1-[3,5-bis(trifluoromethyl)phenyl]ethoxy}-7-(4-fluorophenyl)-2-methyl-3-oxohexahydro-1H-pyrrolizin-2-yl]carbamate (intermediates from Example 25 and 26, step B) according to the procedure for Example 25 and 26, Step D. Isomer 1 and 2: MS: 505 (MH)⁺.

### EXAMPLE 29

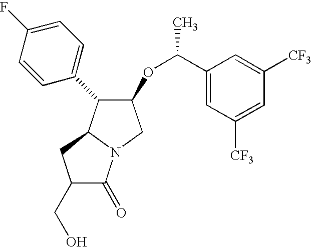

### (6R,7S,7aS)-6-{(1R)-1-[3,5-bis(trifluoromethyl)phenyl]ethoxy}-7-(4-fluorophenyl)-[2-hydroxymethyl]hexahydro-3H-pyrrolizin-3-one

### Step A: (6R,7S,7aS)-6-{(1R)-1-[3,5-bis(trifluoromethyl)phenyl]ethoxy}-7-(4-fluorophenyl)-3-oxohexahydro-1H-pyrrolizine-2-carbaldehyde

To a solution of 300 mg (0.61 mmol) (6R,7S,7aS)-6-{(1R)-1-[3,5-bis(trifluoromethyl)phenyl]ethoxy}-7-(4-fluorophenyl)hexahydro-3H-pyrrolizin-3-one in 3 mL anhydrous THF under nitrogen atmosphere cooled to −78° C. was added dropwise via syringe 765 μL 1.0 M LHMDS in THF and the resulting solution stirred for one hour allowing to warm to −20° C. To the solution was then added 90 μL anhydrous DMF and the mixture stirred for two hours allowing to warm to room temperature. The reaction mixture was quenched with saturated solution of ammonium chloride and extracted with ether (3×10 mL). The organics were combined and treated as stated in Example 3, step E. The residue was purified by preparative TLC plate eluting with ethyl acetate/hexane (9/1) to afford 380 mg (76%) of the title compound as a 1:1 mixture of diastereomers.

### Step B: (6R,7S,7aS)-6-{(1R)-1-[3,5-bis(trifluoromethyl)phenyl]ethoxy}-7-(4-fluorophenyl)-[2-hydroxymethyl]hexahydro-3H-pyrrolizin-3-one

To a solution of 100 mg (0.20 mmol) in 2 mL methanol was added 36 mg (1.0 mmol) sodiumborohydride and the resulting solution stirred for 3 hours at room temperature. The solution was concentrated in vacuo and the residue dissolved in 10 mL dichloromethane. The suspension was washed with saturated sodium bicarbonate solution followed by brine. The organics were dried over magnesium sulfate, filtered through a fritted funnel, and the filtrate evaporated in vacuo. The residue was purified by preparative TLC plate eluting with ethyl acetate to afford 77 mg (79%) of the title compound as a 1:1 mixture of diastereomers. MS: 506 (MH)⁺; 528 (M+Na)⁺.

### EXAMPLE 30

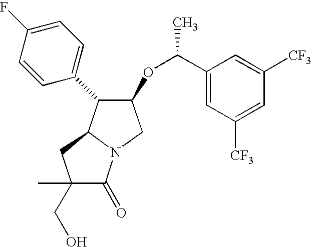

### (6R,7S,7aS)-6-{(1R)-1-[3,5-bis(trifluoromethyl)phenyl]ethoxy}-7-(4-fluorophenyl)-2-(hydroxymethyl)-2-methylhexahydro-3H-pyrrolizin-3-one

### Step A: (6R,7S,7aS)-6-{(1R)-1-[3,5-bis(trifluoromethyl)phenyl]ethoxy}-7-(4-fluorophenyl)-2-(hydroxymethyl)-2-methylhexahydro-3H-pyrrolizin-3-one

To a solution of 500 mg (1.0 mmol) methyl (6R,7S,7aS)-6-{(1R)-1-[3,5-bis(trifluoromethyl)phenyl]ethoxy}-7-(4-fluorophenyl)-2-methyl-3-oxohexahydro-1H-pyrrolizine-2-carboxylate in 8 mL anhydrous dichloromethane under nitrogen atmosphere cooled to −78° C. was added dropwise via syringe 2 mL (2.0 mmol) of 1.0 M DIBAL-H in cyclohexane and the resulting solution stirred for 3 hours at −78° C. The mixture was quenched by pouring the contains over ice/water and then extracting with ethyl acetate (3×20 mL). The organics were combined and treated as stated in Example 3, step E. The residue was purified by preparative TLC eluting with ethyl acetate/hexane (9/1) to afford 280 mg (50%) of the title compound as one single isomer. MS: 520 (MH)⁺; 544 (M+Na)⁺.

### EXAMPLE 31

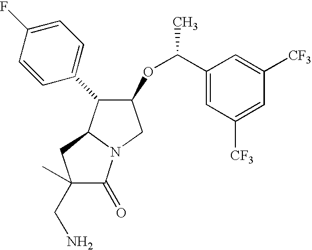

### (6R,7S,7aS)-2-(aminomethyl)-6-{(1R)-1-[3,5-bis(trifluoromethyl)phenyl]ethoxy}-7-(4-fluorophenyl)-2-methylhexahydro-3H-pyrrolizin-3-one

### Step A: (6R,7S,7aS)-6-{(1R)-1-[3,5-bis(trifluoromethyl)phenyl]ethoxy}-7-(4-fluorophenyl)-2-methyl-3-oxohexahydro-1H-pyrrolizine-2-carbaldehyde

To a solution of 500 mg (1.0 mmol) methyl (6R,7S,7aS)-6-{(1R)-1-[3,5-bis(trifluoromethyl)phenyl]ethoxy}-7-(4-fluorophenyl)-2-methyl-3-oxohexahydro-1H-pyrrolizine-2-carboxylate in 8 mL anhydrous dichloromethane under nitrogen atmosphere cooled to −78° C. was added dropwise via syringe 0.95 mL (0.95 mmol) of 1.0 M DIBAL-H in cyclohexane and the resulting solution stirred for 1 hour at −78° C. The mixture was quenched by pouring the contains over ice/water and then extracting with ethyl acetate (3×20 mL). The organics were combined and treated as stated in Example 3, step E. The residue was purified by preparative TLC eluting with ethyl acetate/hexane (9/1) to afford 185 mg (36%) of the title compound as one single isomer.

### Step B: (6R,7S,7aS)-2-[(benzylamino)methyl]-6-{(1R)-1-[3,5-bis(trifluoromethyl)phenyl]ethoxy}-7-(4-fluorophenyl)-2-methylhexahydro-3H-pyrrolizin-3-one

To a solution of 50 mg (0.10 mmol)(6R,7S,7aS)-6-{(1R)-1-[3,5-bis(trifluoromethyl)phenyl]ethoxy}-7-(4-fluorophenyl)-2-methyl-3-oxohexahydro-1H-pyrrolizine-2-carbaldehyde and 11 μL benzylamine in 1 mL dichloromethane was added 25 mg 4A molecular sieves followed by 105 mg (0.50 mmol) sodium triacetoxyborohydride and the resulting suspension stirred vigorously overnight at room temperature. The mixture was quenched with saturated solution of sodium bicarbonate (3 mL) and extracted with dichloromethane (3×5 mL). The organics were combined and treated as stated in Example 3, step E. The residue was purified by preparative TLC plate eluting with dichloromethane/methanol (9/1) to afford 47 mg (80%) of the title compound as one single isomer. MS: 609 (MH)⁺;

### Step C: (6R,7S,7aS)-2-(aminomethyl)-6-{(1R)-1-[3,5-bis(trifluoromethyl)phenyl]ethoxy}-7-(4-fluorophenyl)-2-methylhexahydro-3H-pyrrolizin-3-one

The title compounds were prepared from (6R,7S,7aS)-2-[(benzylamino)methyl]-6-{(1R)-1-[3,5-bis(trifluoromethyl)phenyl]ethoxy}-7-(4-fluorophenyl)-2-methylhexahydro-3H-pyrrolizin-3-one according to the procedure for Example 25 and 26, Step D. MS: 519 (MH)⁺.

### EXAMPLE 32

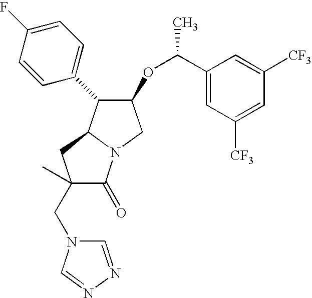

### (6R,7S,7aS)-6-{(1R)-1-[3,5-bis(trifluoromethyl)phenyl]ethoxy}-7-(4-fluorophenyl)-2-methyl-2-4H-1,2,4-triazol-4-ylmethyl)hexahydro-3H-pyrrolizine-3-one

### Step A: (6R,7S,7aS)-6-{(1R)-1-[3,5-bis(trifluoromethyl)phenyl]ethoxy}-7-(4-fluorophenyl)-2-methyl-2-(4H-1,2,4-triazol-4-ylmethyl)hexahydro-3H-pyrrolizin-3-one

The title compound was prepared from (6R,7S,7aS)-2-(aminomethyl)-6-{(1R)-1-[3,5-bis(trifluoromethyl)phenyl]ethoxy}-7-(4-fluorophenyl)-2-methylhexahydro-3H-pyrrolizin-3-one (intermediate Example 27, step B) according to the procedure for Example 20, Step A. MS: 571 (MH)⁺.

### EXAMPLE 33

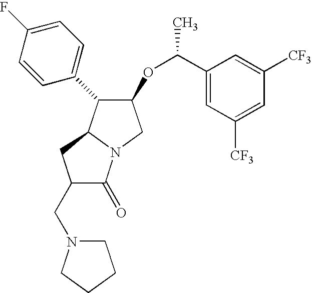

### (6R,7S,7aS)-6-{(1R)-1-[3,5-bis(trifluoromethyl)phenyl]ethoxy}-7-(4-fluorophenyl)-2-(pyrrolidine-1-ylmethyl)hexahydro-3H-pyrrolizin-3-one

### Step A: (6R,7S,7aS)-6-{(1R)-1-[3,5-bis(trifluoromethyl)phenyl]ethoxy}-7-(4-fluorophenyl)-2-(pyrrolidine-1-ylmethyl)hexahydro-3H-pyrrolizin-3-one

The title compound was prepared from (6R,7S,7aS)-6-{(1R)-1-[3,5-bis(trifluoromethyl)phenyl]ethoxy}-7-(4-fluorophenyl)-3-oxohexahydro-1H-pyrrolizine-2-carbaldehyde (intermediate Example 27, step A) according to the procedure for Example 31, Step A. MS: 559 (MH)⁺.

Using the procedures similar to those described above the following Examples were prepared.

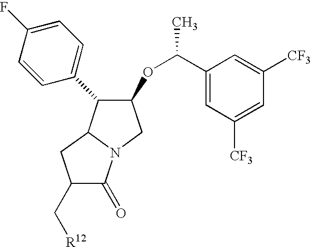

| | R¹² | parent ion |
|---|---|---|
| Example 34 | 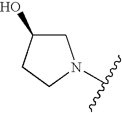 | 575 |
| Example 35 | 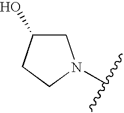 | 575 |

### EXAMPLE 36

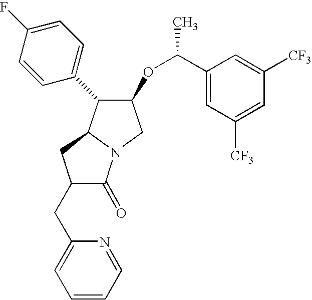

### (6R,7S,7aS)-6-{(1R)-1-[3,5-bis(trifluoromethyl)phenyl]ethoxy}-7-(4-fluorophenyl)-2-(pyridine-2-ylmethyl)hexahydro-3H-pyrrolizin-3-one

### Step A: (6R,7S,7aS)-6-{(1R)-1-[3,5-bis(trifluoromethyl)phenyl]ethoxy}-7-(4-fluorophenyl)-2-(pyridine-2-ylmethyl)hexahydro-3H-pyrrolizin-3-one

To a solution of 40 mg (0.08 mmol) (6R,7S,7aS)-6-{(1R)-1-[3,5-bis(trifluoromethyl)phenyl]ethoxy}-7-(4-fluorophenyl)hexahydro-3H-pyrrolizin-3-one in 2 mL anhydrous THF set under nitrogen atmosphere cooled to −78° C. was added via syringe dropwise 84 μL (0.16 mmol) of 2.0 M LDA in THF/cyclohexane/ethylbenzene and the resulting solution stirred for one hour allowing to warm to −20° C. Once the solution attained −20° C., 28 mg (0.16 mmol) 2-Picolyl chloride in 0.5 mL THF was added via syringe and the solution stirred for two hours allowing to warm to room temperature. The reaction was quenched with saturated solution of ammonium chloride and extracted with dichloromethane (2×10 mL). The organics were combined and treated as stated in Example 3, step E. The residue was purified by preparative TLC plate eluting with dichloromethane/methanol (9/1) to afford 6 mg (15%) of the title compound as a (1:1) mixture of diastereomers. MS: 567 (MH)⁺.

Using the procedures similar to those described above the following Examples were prepared.

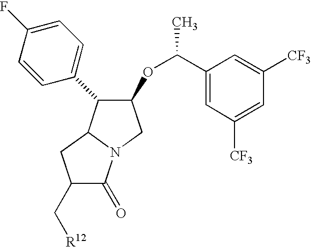

| | R¹² | parent ion |
|---|---|---|
| Example 37 | 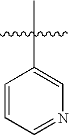 | 567 |
| Example 38 | 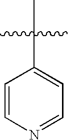 | 567 |
| Example 39 | 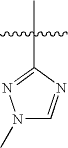 | 571 |

### EXAMPLE 40

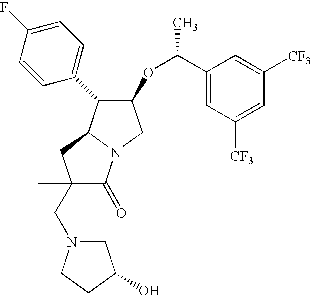

### (6R,7S,7aS)-6-{(1R)-1-[3,5-bis(trifluoromethyl)phenyl]ethoxy}-7-(4-fluorophenyl)-2-{[(3R)-3-hydroxypyrrolidin-1-yl]methyl}-2-methylhexahydro-3H-pyrrolizin-3-one

### Step A: (6R,7S,7aS)-6-{(1R)-1-[3,5-bis(trifluoromethyl)phenyl]ethoxy}-7-(4-fluorophenyl)-2-{[(3R)-3-hydroxypyrrolidin-1-yl]methyl}-2-methylhexahydro-3H-pyrrolizin-3-one

The title compound was prepared from (6R,7S,7aS)-6-{(1R)-1-[3,5-bis(trifluoromethyl)phenyl]ethoxy}-7-(4-fluorophenyl)-2-methyl-3-oxohexahydro-1H-pyrrolizine-2-carbaldehyde (intermediate Example 29, step A) according to the procedure for Example 31, Step B. MS: 589 (MH)⁺.

Using the procedures similar to those described above the following Examples were prepared.

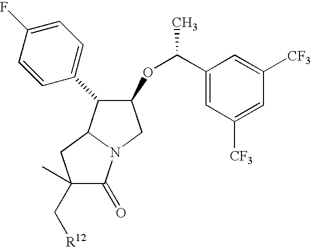

| | R¹² | parent ion |
|---|---|---|
| Example 41 | 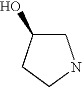 | 589 |
| Example 42 | 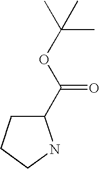 | 673617 [M − (t-Bu)]⁺ |
| Example 43 | 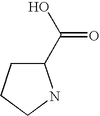 | 617 |
| Example 44 | 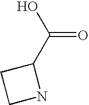 | 603 |

### EXAMPLE 45 and 46

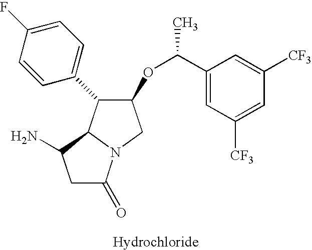

### (6R,7S,7aR)-(1R or S)-amino-6-{(1R)-1-[3,5-bis(trifluoromethyl)phenyl]ethoxy}-7-(4-fluorophenyl)hexahydro-3H-pyrrolizin-3-one hydrochloride

### Step A: tert-butyl (2R,3S,4R)-4-{(1R)-1-[3,5-bis(trifluoromethyl)phenyl]ethoxy}-3-(4-fluorophenyl)-2-formylpyrrolidine-1-carboxylate

To a stirred solution of 0.68 mL (1.36 mmol) oxalyl chloride in 15 mL dry methylene chloride under nitrogen atmosphere at −78° C. was added 0.19 mL (2.72 mmol) DMSO dropwise over 5 min by syringe. After ten min., to this mixture was added a solution of 375 mg (0.68 mmol) tert-butyl 4-{1-[3,5-bis(trifluoromethyl)phenyl]ethoxy}-2-(hydroxymethyl)-3-phenylpyrrolidine-1-carboxylate (Example 1) in 1 mL dry methylene chloride. The reaction mixture was stirred at −78° C. for 1 hr, then 0.76 mL (5.44 mmol) TEA was added by syringe. The reaction mixture was stirred at −78° C. for 15 min then warmed to room temperature and stirred an additional hr. The reaction mixture was quenched with aq. 1N HCL (˜15 mL) and transferred to a separatory funnel. The reaction mixture was extracted with EtOAc (2×15 mL). The combined organic extracts were washed with water (15 mL) then brine (15 mL), dried over anhydrous sodium sulfate, filtered and evaporated under vacuum afford the title compound. The resulting crude product was purified by preparative TLC plate eluding with ethyl acetate/hexane (1/4) to afford 350 mg (95%) of the title compound.

### Step B: tert-butyl (2R,3S,4R)-4-{(1R)-1-[3,5-bis(trifluoromethyl)phenyl]ethoxy}-2-{(E)-[(tert-butylsulfinyl)imino]methyl}-3-(4-fluorophenyl)pyrrolidine-1-carboxylate

To a solution of 200 mg (0.36 mmol) tert-butyl (2R,3S,4R)-4-{(1R)-1-[3,5-bis(trifluoromethyl)phenyl]ethoxy}-3-(4-fluorophenyl)-2-formylpyrrolidine-1-carboxylate in 2 mL anhydrous dichloromethane under nitrogen atmosphere was added 40 mg (0.34 mmol) (S)-(−)-2-methyl-2-propanesulfimine followed directly by 110 mg (0.72 mmol) solid anhydrous copper sulfate and the resulting suspension was stirred for 18 hours at room temperature. The solid copper sulfate was filtered off through a celite plug and washed with dichloromethane (2×10 mL). The organics were combined and treated as stated in Example 3, step E. The residue was purified by preparative TLC plate eluting with ethyl acetate/hexane (1/4) to afford 108 mg (53%) of the title compound. MS: 652 (MH)⁺; 552 (M−100; M-Boc)⁺.

### Step C: tert-butyl (2R,3S,4R)-4-{(1R)-1-[3,5-bis(trifluoromethyl)phenyl]ethoxy}-2-{3-tert-butoxy-1-[(tert-butylsulfinly)amino]-3-oxopropyl}-3-(4-fluorophenyl)pyrrolidine-1-carboxylate

To a solution of 31 μL (0.23 mmol) tert-butyl acetate in 2 mL anhydrous ether cooled to −78° C. by dry ice/acetone bath under nitrogen atmosphere was added via syringe 153 μL (0-31 mmol) 2.0M sodium bis(trimethylsylyl)amide in THF and the resulting solution stirred for one hour. tert-Butyl (2R,3S,4R)-4-{(1R)-1-[3,5-bis(trifluoromethyl)phenyl]ethoxy}-2-{(E)-[(tert-butylsulfinyl)imino]methyl}-3-(4-fluorophenyl)pyrrolidine-1-carboxylate (100 mg, 0.15 mmol) in 1 mL anhydrous ether was then introduced via syringe to the cooled solution and the resulting mixture was stirred for two hours allowing to warm slowly to room temperature. The mixture was quenched with saturated solution of ammonium chloride and extracted with ether (2×10 mL). The organics were combined and treated as stated in Example 3, step E. The residue was purified by preparative TLC plate eluting with ethyl acetate/hexane (1/4) to afford two separate single diastereomers which were labeled D1 (less polar, 52 mg) and D2 (more polar, 40 mg). MS: 769 (MH)⁺; 669 (M−100; M-Boc)⁺.

### Step D: (3R or S)-amino-3-[(2R,3S,4R)-4-{(1%)-1-[3,5-bis(trifluoromethyl)phenyl]ethoxy}-3-(4-fluorophenyl)pyrrolidin-2-yl]propanoic acid di-hydrochloride (D1, less polar)

The title compound was prepared by treatment of the less polar diastereomer of tert-butyl (2R,3S,4R)-4-{(1R)-1-[3,5-bis(trifluoromethyl)phenyl]ethoxy}-2-{3-tert-butoxy-1-[(tert-butylsulfinly)amino]-3-oxopropyl}-3-(4-fluorophenyl)pyrrolidine-1-carboxylate (diastereomer D1 of step C) with 3 mL of 4N HCl in dioxane for one hour at room temperature. Concentration in vacuo afforded 45 mg of the title compound with no further purification necessary. ¹H NMR (500 MHz, CD₃OD): δ 8.00 (s, 1H), 7.80 (s, 1H), 7.69 (s, 2H), 7.30 (app q, J=5.2 Hz, 2H), 7.06 (app t, J=8.7 Hz, 2H), 4.74 (q, J=6.4 Hz, 1H), 4.17-4.10 (m, 2H), 3.90-3.80 (m, 2H), 3.63 (dd, J=4.3, 12.4 Hz, 1H), 3.46 (dd, J=6.2, 11.0 Hz, 1H) 3.02 (s, 1H), 2.90 (dd, J=4.6, 18.0 Hz, 1H) 2.88 (s, 1H), 2.80 (dd, J=7.7, 18.0 Hz, 1H) 2.18 (s, 2H), 1.45 (d, J=6.4 Hz, 3H). MS: 609 (MH)⁺.

### Step E: (3R or S)-amino-3-[(2R,3S,4R)-4-{(1R)-1-[3,5-bis(trifluoromethyl)phenyl]ethoxy}-3-(4-fluorophenyl)pyrrolidin-2-yl]propanoic acid dihydrochloride (D2, more polar)

The title compound was prepared from the more polar diastereomer of tert-butyl (2R,3S,4R)-4-{(1R)-1-[3,5-bis(trifluoromethyl)phenyl]ethoxy}-2-{3-tert-butoxy-1-[(tert-butylsulfinly)amino]-3-oxopropyl}-3-(4-fluorophenyl)pyrrolidine-1-carboxylate (diastereomer D2 of step C) according to the procedure for Example 45 and 46, Step D. ¹H NMR (500 MHz, CD₃OD): δ 8.00 (s, 1H), 7.79 (s, 1H), 7.65 (s, 2H), 7.28 (app q, J=5.2 Hz, 2H), 7.04 (app t, J=8.7 Hz, 2H), 4.71 (q, J=6.4 Hz, 1H), 4.18 (dd, J=7.7, 10.5 Hz, 1H), 4.04 (app q, 6.7 Hz, 1H), 3.94 (app q, 6.1 Hz, 1H), 3.82 (dd, J=6.3, 12.0 Hz, 1H), 3.53 (dd, J=5.3, 12.3 Hz, 1H), 3.45 (dd, J=6.6, 11.0 Hz, 1H) 3.02 (s, 1H), 2.88 (s, 1H), 2.72 (dd, J=4.8, 18.3 Hz, 1H) 2.55 (dd, J=6.2, 18.3 Hz, 1H) 2.18 (s, 2H), 1.43 (d, J=6.4 Hz, 3H). MS: 609 (MH)⁺.

### Step F: (6R,7S,7aR)-(1R or S)-amino-6-{(1R)-1-[3,5-bis(trifluoromethyl)phenyl]ethoxy}-7-(4-fluorophenyl)hexahydro-3H-pyrrolizin-3-one hydrochloride (isomer D1)

To a solution of 20 mg (D1, 0.35 mmol) (3R or S)-amino-3-[(2R,3S,4R)-4-{(1R)-1-[3,5-bis(trifluoromethyl)phenyl]ethoxy}-3-(4-fluorophenyl)pyrrolidin-2-yl]propanoic acid di-hydrochloride (intermediate from step D) and 13 μL N,N-diisopropyl-N-ethyl amine (DIEA, 0.70 mmol) in 1 mL dichloromethane was added 1 mg DMAP followed by 14 mg (0.70 mmol) EDC and the resulting solution was stirred overnight at room temperature. The solution was concentrated in vacuo and the residue purified by preparative TLC plate eluting with 1% ammonium hydroxide/10% methanol/89% dichloromethane to afford 10 mg (60%) of the title compound. The compound was then treated with 50 μL 4N HCl dioxane in 1 mL dichloromethane and concentrated in vacuo to convert it to the HCl salt. ¹H NMR (500 MHz, CD₃OD): δ 7.78 (s, 1H), 7.62 (s, 2H), 7.28-7.24 (m, 2H), 7.02 (app t, J=8.7 Hz, 2H), 4.68 (q, J=6.7 Hz, 1H), 4.26-4.21 (m, 1H), 4.00-3.92 (m, 2H), 3.78 (dd, J=4.6, 12.1 Hz, 1H), 3.64 (dd, J=7.0, 12.1 Hz, 1H), 3.12-3.05 (m, 2H), 2.75 (dd, J=5.8, 17.2 Hz, 1H), 1.38 (d, J=6.4 Hz, 3H). MS: 491 (MH)⁺.

### Step G: (6R,7S,7aR)-(1R or S)-amino-6-{(1R)-1-[3,5-bis(trifluoromethyl)phenyl]ethoxy}-7-(4-fluorophenyl)hexahydro-3H-pyrrolizin-3-one hydrochloride (isomer D2)

The title compound was prepared from the more polar diastereomer of (3R or S)-amino-3-[(2R,3S,4R)-4-{(1R)-1-[3,5-bis(trifluoromethyl)phenyl]ethoxy}-3(4-fluorophenyl)pyrrolidin-2-yl]propanoic acid di-hydrochloride (intermediate from step E) according to the procedure for Example 43 and 44, Step F. ¹H NMR (500 MHz, CD₃OD): δ 7.78 (s, 1H), 7.60 (s, 2H), 7.34-7.28 (m, 2H), 7.03 (app t, J=8.7 Hz, 2H), 4.67 (q, J=6.4 Hz, 1H), 4.56 (dd, J=5.5, 11.3 Hz, 1H), 4.25 (app q, J=7.1, 11H), 4.08 (app t, J=5.8 Hz, 1H), 3.80 (dd, J=7.6, 11.2 Hz, 1H), 3.52 (dd, J=6.5, 11.6 Hz, 1H), 3.25 (dd, J=6.9, 17.6 Hz, 1H), 3.12 (dd, J=8.2, 11.2 Hz, 1H), 2.45 (d, J=17.6 Hz, 1H) 1.42 (d, J=6.4 Hz, 3H). MS: 491 (MH)⁺.

### EXAMPLE 47 and 48

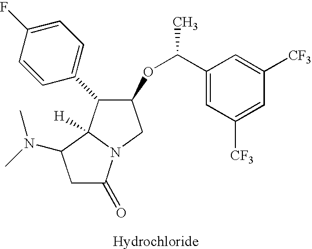

### 6R,7S,7aR)-6-{(1R)-1-[3,5-bis(trifluoromethyl)phenyl]ethoxy}-1(R or S)-(dimethylamino)-7-(4-fluorophenyl)hexahydro-3H-pyrrolizine-3-one hydrochloride

### Step A: (6R,7S,7aR)-6-{(1R)-1-[3,5-bis(trifluoromethyl)phenyl]ethoxy}-1(R or S)-(dimethylamino)-7-(4-fluorophenyl)hexahydro-3H-pyrrolizine-3-one hydrochloride (isomer D1)

The title compound was prepared from (6R,7S,7aR)-(1R or S)-amino-6-{(1R)-1-[3,5-bis(trifluoromethyl)phenyl]ethoxy}-7-(4-fluorophenyl)hexahydro-3H-pyrrolizine-3-one hydrochloride (isomer D1 from Example 45 and 46, Step F) according to the procedure for Example 8. MS: 519 (MH)⁺.

### Step B: (6R,7S,7aR)-6-{(1R)-1-[3,5-bis(trifluoromethyl)phenyl]ethoxy}-1(R or S)-(dimethylamino)-7-(4-fluorophenyl)hexahydro-3H-pyrrolizine-3-one hydrochloride (isomer D2)

The title compound was prepared from (6R,7S,7aR)-(1R or S)-amino-6-{(1R)-1-[3,5-bis(trifluoromethyl)phenyl]ethoxy}-7-(4-fluorophenyl)hexahydro-3H-pyrrolizine-3-one hydrochloride (isomer D2 from Example 45 and 46, Step G) according to the procedure for Example 8. MS: 519 (MH)⁺.

### EXAMPLE 49

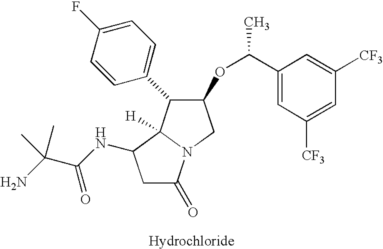

### N¹-[(6R,7S,7aR)-6-{(1R)-1-[3,5-bis(trifluoromethyl)phenyl]ethoxy}-7-(4-fluorophenyl)-3-oxohexahydro-1H-pyrrolizin-1-yl]-2-methylalaninamide hydrochloride

### Step A: (t-butoxycarbonyl)-N¹-[(6R,7S,7aR)-6-{(1R)-1-[3,5-bis(trifluoromethyl)phenyl]ethoxy}-7-(4-fluorophenyl)-3-oxohexahydro-1H-pyrrolizin-1-yl]-2-methylalaninamide (isomer D1)

To a solution of 5 mg (D1, 0.01 mmol) (6R,7S,7aR)-(1R or S)-amino-6-{(1R)-1-[3,5-bis(trifluoromethyl)phenyl]ethoxy}-7-(4-fluorophenyl)hexahydro-3H-pyrrolizin-3-one hydrochloride (isomer D1 from Example 45 and 46, step A), 2 μL N,N-diisopropyl-N-ethyl amine (DIEA, 0.01 mmol), 0.5 mg DMAP, and 4 mg (0.02 mmol) in 1 mL dichloromethane was added 4 mg (0.02 mmol) EDC and the resulting solution was stirred overnight at room temperature. The solution was concentrated in vacuo and the residue purified by preparative TLC plate eluting with methanol/dichloromethane (1/9) to afford 5.2 mg (85%) of the title compound. MS: 676 (MH)⁺; 576 [M-100 (Boc)]⁺.

### Step B: N¹-[(6R,7S,7aR)-6-{(1R)-1-[3,5-bis(trifluoromethyl)phenyl]ethoxy}-7-(4-fluorophenyl)-3-oxohexahydro-1H-pyrrolizin-1-yl]-2-methylalaninamide hydrochloride (isomer D1)

The title compound was prepared by treatment of the less polar diastereomer of (t-butoxycarbonyl)-N′-[(6R,7S,7aR)-6-{(1R)-1-[3,5-bis(trifluoromethyl)phenyl]ethoxy}-7-(4-fluorophenyl)-3-oxohexahydro-1H-pyrrolizin-1-yl]-2-methylalaninamide(isomer D1, intermediate from step A) with 1 mL of 4N HCl in dioxane for one hour at room temperature. Concentration in vacuo afforded 3.8 mg of the title compound with no further purification necessary. MS: 576 (MH)⁺.

Using the procedures similar to those described above the following Examples were prepared.

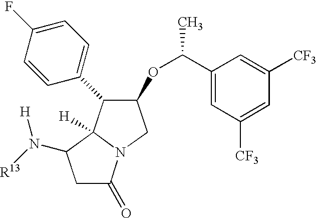

| | R¹³ | parent ion |
|---|---|---|
| Example 50Isomer D2 | 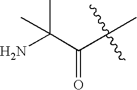 | 576 |
| Example 51Isomer D1(used only Step Aprocedure) | 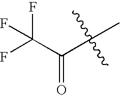 | 587 |
| Example 52Isomer D2(used only Step Aprocedure) | 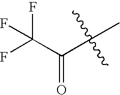 | 587 |

### EXAMPLE 53 and 54

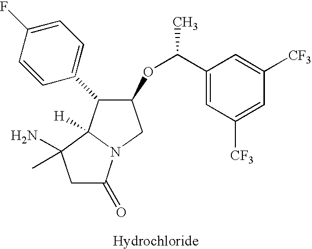

### (6R,7S,7aR)-(1R or S)-amino-6-{(1R)-1-[3,5-bis(trifluoromethyl)phenyl]ethoxy}-7-(4-fluorophenyl) 1-methylhexahydro-3H-pyrrolizin-3-one hydrochloride

### Step A: tert-butyl (2R,3S,4R)-4-{(1R)-1-[3,5 bis(trifluoromethyl)phenyl]ethoxy}-3-(4-fluorophenyl)-2-(1-hydroxyethyl)pyrrolidine-1-carboxylate

To a solution of 350 mg (0.64 mmol) tert-butyl (2R,3S,4R)-4-{(1R)-1-[3,5-bis(trifluoromethyl)phenyl]ethoxy}-3-(4-fluorophenyl)-2-formylpyrrolidine-1-carboxylate in 10 mL anhydrous toluene under nitrogen atmosphere cooled to 0° C. by ice/water bath was added via syringe 2.28 mL 1.4 M methylmagnesium bromide in THF and the resulting solution stirred for two hours. The reaction was quenched with saturated solution of ammonium chloride and extracted with ethyl acetate (2×20 mL). The organics were combined and treated as stated in Example 3, step E. The residue was purified by preparative TLC plate eluting with ethyl acetate/methanol (9/1) to afford 325 mg (90%) of the title compound as a mixture of diastereomers. MS: 566 (MH)⁺; 588 (M+Na)⁺.

### Step B: tert-butyl (2R,3S,4R)-2-acetyl-4-{(1R)-1-[3,5bis(trifluoromethyl)phenyl]ethoxy}-3-(4-fluorophenyl)pyrrolidine-1-carboxylate

The title compound was prepared from tert-butyl (2R,3S,4R)-4-{(1R)-1-[3,5bis(trifluoromethyl)phenyl]ethoxy}-3-(4-fluorophenyl)-2-(1-hydroxyethyl)pyrrolidine-1-carboxylate (intermediate from step A) according to the procedure for Example 3, Step A. MS: 564 (MH)⁺; 586 (M+Na)⁺.

### Step C: tert-butyl (2R,3S,4R)-4-{(1R)-1-[3,5bis(trifluoromethyl)phenyl]ethoxy}-2-[(1E)-N-(tert-butylsulfinyl)ethanimidoyl]-3-(4-fluorophenyl)pyrrolidine-1-carboxylate

To a solution of 140 mg (0.25 mmol) tert-butyl (2R,3S,4R)-2-acetyl-4-{(1R)-1-[3,5bis(trifluoromethyl)phenyl]ethoxy}-3-(4-fluorophenyl)pyrrolidine-1-carboxylate (intermediate from step B) in 1 mL anhydrous THE was added 30 mg (0.26 mmol) (S)-(−)-2-methyl-2-propanesulfimine followed by 114 μL titanium ethoxide and the resulting solution was set under nitrogen atmosphere and refluxed for 15 hours. The mixture was cooled to room temperature and concentrated in vacuo. The residue was purified by preparative TLC plate eluting with ethyl acetate/hexane (1/1) to afford 123 mg (77%) of the title compound. MS: 666 (MH)⁺; 566 (M−100; M-Boc)⁺.

### Step D: tert-butyl (2R,3S,4R)-4-{(1R)-1-[3,5bis(trifluoromethyl)phenyl]ethoxy}-2-{1-[(tert-butylsulfinyl)amino]-1-methylbut-3-en-1-yl}-3-(4-fluorophenyl)pyrrolidine-1-carboxylate

To a solution of 100 mg (0.15 mmol) tert-butyl (2R,3S,4R)-4-{(1R)-1-[3,5bis(trifluoromethyl)phenyl]ethoxy}-2-[(1E)-N-(tert-butylsulfinyl)ethanimidoyl]-3-(4-fluorophenyl)pyrrolidine-1-carboxylate (intermediate from step C) in 1 mL anhydrous THF cooled to 0° C. under nitrogen atmosphere was added dropwise via syringe 450 μL 1.0M allylmagnesium bromide in ether and the resulting solution was stirred for one hour at 0° C. The reaction was quenched with brine and extracted with ether (2×5 mL). The organics were combined and treated as stated in Example 3, step E. The residue was purified by preparative TLC plate eluting with ethyl acetate/hexane (1/1) to afford 40 mg (38%) of the title compound as one single diastereomer. MS: 709 (MH)⁺; 609 (M−100 μM-Boc)⁺.

### Step E: tert-butyl (2R,3S,4R)-4-{(1R)-1-[3,5bis(trifluoromethyl)phenyl]ethoxy}-2-{1-[(tert-butylsulfinyl)amino]-1-methyl-3-oxopropyl}-3-(4-fluorophenyl)pyrrolidine-1-carboxylate

To a solution of 38 mg (0.06 mmol) tert-butyl (2R,3S,4R)-4-{(1R)-1-[3,5 bis(trifluoromethyl)phenyl]ethoxy}-2-{1-[(tert-butylsulfinyl)amino]-1-methylbut-3-en-1-yl}-3-(4-fluorophenyl)pyrrolidine-1-carboxylate (intermediate from step D) in 2 mL dichloromethane cooled to −78° C. by dry ice/acetone bath was bubbled ozone for 1 minute until a blue color persisted. To get rid of the excess ozone, nitrogen gas was then bubbled into the solution until it became clear. The solution warmed to room temperature and 28 mg (0.11 mmol) triphenylphosphine was then added to the solution to reduce the ozonide. After one hour stirring at room temperature, the reaction was proven to be complete by checking LC-MS and TLC. The mixture was concentrated in vacuo and the residue purified by preparative TLC plate eluting with ethyl acetate/hexane (3/2) to afford 26 mg (68%) of the title compound.

### Step F: 3-[(2R,3S,4R)-4-{(1R)-1-[3,5bis(trifluoromethyl)phenyl]ethoxy}-1-(tert-butoxycarbonyl)-3-(4-fluorophenyl)pyrrolidin-2-yl]-3-[(tert-butylsulfinyl)amino]butanoic acid

To a solution of 25 mg (0.04 mmol) tert-butyl (2R,3S,4R)-4-{(1R)-1-[3,5bis(trifluoromethyl)phenyl]ethoxy}-2-{1-[(tert-butylsulfinyl)amino]-1-methyl-3-oxopropyl}-3-(4-fluorophenyl)pyrrolidine-1-carboxylate (intermediate from step E) in 1.25 mL of tert-butanol/2-methylbut-2-ene (4/1) was added NaH₂PO₄ (32 mg, 0.23 mmol) and NaClO₂ (28 mg, 0.30 mmol) in 0.5 mL water and the resulting solution was stirred for one hour at room temperature. The reaction was diluted with dichloromethane (2 mL), separated, and the aqueous then extracted with dichloromethane (2×2 mL). The organics were combined and treated as stated in Example 3, step E. The residue was purified by preparative TLC plate eluting with methanol/dichloromethane (1/9) to afford 20 mg (79%) of the title compound. MS: 727 (MH)⁺; 627 (M-100; M-Boc)⁺.

### Step G: 3-amino-3-[(2R,3S,4R)-4-{(1R)-1-[3,5-bis(trifluoromethyl)phenyl]ethoxy}-3-(4-fluorophenyl)pyrrolidin-2-yl]butanoic acid dihydrochloride

The title compound was prepared by treatment of the less polar diastereomer 3-[(2R,3S,4R)-4-{(R)-1-[3,5bis(trifluoromethyl)phenyl]ethoxy}-1-(tert-butoxycarbonyl)-3-(4-fluorophenyl)pyrrolidin-2-yl]-3-[(tert-butylsulfinyl)amino]butanoic acid (intermediate from step F) with 2 mL of 4N HCl in dioxane for one hour at room temperature. Concentration in vacuo afforded 15 mg of the title compound with no further purification necessary. MS: 523 (MH)⁺.

### Step H: (6R,7S,7aR)-(1R or S)-amino-6-{(1R)-1-[3,5-bis(trifluoromethyl)phenyl]ethoxy}-7-(4-fluorophenyl)-1-methylhexahydro-3H-pyrrolizin-3-one hydrochloride

The title compound was prepared from 3-amino-3-[(2R,3S,4R)-4{(1R)-1-[3,5-bis(trifluoromethyl)phenyl]ethoxy}-3-(4-fluorophenyl)pyrrolidin-2yl]butanoic acid dihydrochloride (intermediate from step G) according to the procedure for Example 45 and 46, Step F. ¹H NMR (500 MHz, CD₃OD): δ 7.78 (s, 1H), 7.56 (s, 2H), 7.34-7.28 (m, 2H), 7.04 (app t, J=8.7 Hz, 2H), 4.65 (q, J=6.4 Hz, 1H), 4.36 (d, J=11.2 Hz, 1H), 4.26 (app q, J=7.1, 1H), 3.83 (dd, J=7.6, 11.5 Hz, 1H), 3.48 (dd, J=6.9, 11.2 Hz, 1H), 3.07 (d, J=17.4 Hz, 1H) 3.02 (overlapping dd, J=8.3, 11.0 Hz, 1H), 2.53 (d, J=17.4 Hz, 1H) 1.41 (d, J=6.4 Hz, 3H), 1.30 (s, 3H). MS: 505 (MH)⁺;

### EXAMPLE 55

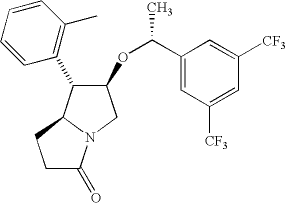

### (6R,7S,7aS)-6-{(1R)-1-[3,5-bis(trifluoromethyl)phenyl]ethoxy}-7-(2-methylphenyl)hexahydro-3H-pyrrolizin-3-one

The title compounds were prepared as in example 2. MS: 472 (M+1).

### EXAMPLE 56 and 57

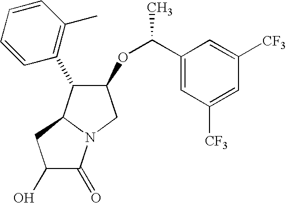

### (6R,7S,7aS)-6-{(1R)-1-[3,5-bis(trifluoromethyl)phenyl]ethoxy}-2(R or S)-hydroxy-7-(2-methylphenyl)hexahydro-3H-pyrrolizin-3-one

The title compounds were prepared from (6R,7S,7aS)-6-{(R)-1-[3,5-bis(trifluoromethyl)phenyl]ethoxy}-7-(2-methylphenyl)hexahydro-3H-pyrrolizin-3-one as in example 4.

Isomer 1 was named for the less polar isomer, MS: 488 (M+H).

Isomer 2 was named for the more polar isomer, MS: 488 (M+H).

### EXAMPLE 58

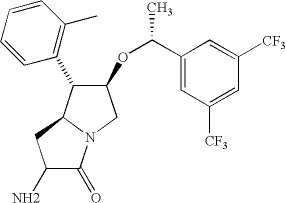

### (6R,7S,7aS)-2(R or S)-amino-6-{(1R)-1-[3,5-bis(trifluoromethyl)phenyl]ethoxy}-7-(2-methylphenyl)hexahydro-3H-pyrrolizin-3-one

The title compound was prepared from the less polar isomer of (6R,7S,7aS)-6-{(1R)-1-[3,5-bis(trifluoromethyl)phenyl]ethoxy}-2-hydroxy-7-(2-methylphenyl)hexahydro-3H-pyrrolizin-3-one. MS: 487 (M+1).

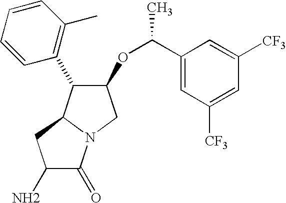

### EXAMPLE 59

### (6R,7S,7aS)-2(R or S)-amino-6-{(1R)-1-[3,5-bis(trifluoromethyl)phenyl]ethoxy}-7-(2-methylphenyl)hexahydro-3H-pyrrolizin-3-one

The title compound was prepared from the more polar isomer of (6R,7S,7aS)-6-{(1R)-1-[3,5-bis(trifluoromethyl)phenyl]ethoxy}-2-hydroxy-7-(2-methylphenyl)hexahydro-3H-pyrrolizin-3-one. MS: 487 (M+1).

### EXAMPLE 60 and EXAMPLE 61

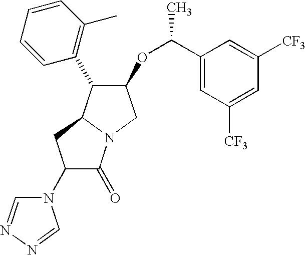

### (6R,7S,7aS)-6-{(1R)-1-[3,5-bis(trifluoromethyl)phenyl]ethoxy}-7-(2-methylphenyl)-2 (R or S)-(4H-1,2,4-triazol-4-yl)hexahydro-3H-pyrrolizin-3-one

The title compounds were prepared from amines in examples 58 and 59 by the method in example 22, respectively. MS: 539 (M+1).

### EXAMPLE 62

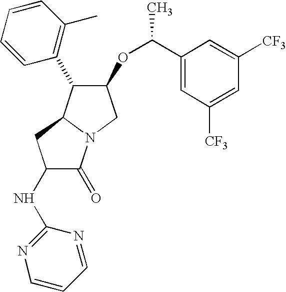

### (6R,7S,7aS)-6-{(1R)-1-[3,5-bis(trifluoromethyl)phenyl]ethoxy}-7-(2-methylphenyl)-2-(pyrimidin-2-ylamino)hexahydro-3H-pyrrolizin-3-one

A solution of the amine (16 mg, example 58) in 3 mL of CH₃OH was added Et₃N (0.046 mL) and 2-Chloropyrimidine (18.8 mg). The mixture in a sealed tube was heated in a 146° C. oil bath for 24 h. Upon removal of volatiles, the crude was purified by reverse phase HPLC to afford the title compound. MS: 565 (M+1).

### EXAMPLE 63

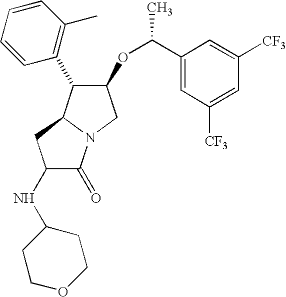

### (6R,7S,7aS)-6-{(1R)-1-[3,5-bis(trifluoromethyl)phenyl]ethoxy}-7-(2-methylphenyl)-2-(tetrahydro-2H-pyran-4-ylamino)hexahydro-3H-pyrrolizin-3-one

The title compound was prepared from the amine (example 58) by reductive amination. MS: 571 (M+1).

### EXAMPLE 64

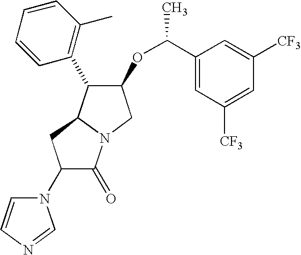

### (6R,7S,7aS)-6-{(1R)-1-[3,5-bis(trifluoromethyl)phenyl]ethoxy}-2-(1H-imidazol-1-yl)-7-(2-methylphenyl)hexahydro-3H-pyrrolizin-3-one

A solution of the amine (11.4 mg, example 58) in NH₄OH (0.5 mL) was added glyoxal (1.07 mL, 40% in water), CH₃OH (0.5 mL) and formaldehyde (0.57 mL, 37% in water). The mixture was heated in a 70° C. oil bath for 3.5 h. Upon removal of volatiles, the crude was purified by reverse phase HPLC to afford the title compound. MS: 538 (M+1).

### EXAMPLE 65

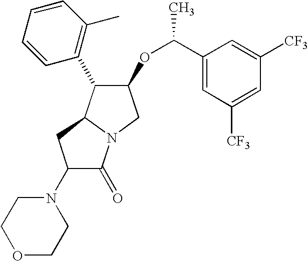

### (6R,7S,7 as)-6-{(1R)-1-[3,5-bis(trifluoromethyl)phenyl]ethoxy}-7-(2-methylphenyl)-2-morpholin-4-ylhexahydro-3H-pyrrolizin-3-one

A solution of the amine (10.9 mg, example 58) in 3 mL of CH₃CN was added 1-bromo-3-(2-bromoethoxy)propane (31 mg), Na₂CO₃. The mixture was heated in a 96° C. oil bath for 40 h. The mixture was diluted with CH₂Cl₂ and was filtered through a plug of celite. The crude was purified by reverse phase HPLC to afford the title compound. MS: 557 (M+1).

### EXAMPLE 66

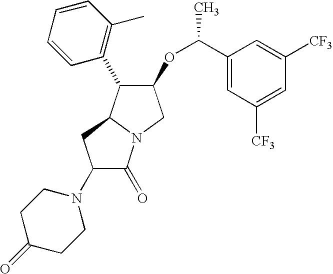

### (6R,7S,7aS)-6-{(1R)-1-[3,5-bis(trifluoromethyl)phenyl]ethoxy}-7-(2-methylphenyl)-2-(4-oxopiperidin-1-yl)hexahydro-3H-pyrrolizin-3-one

A solution of 20 mg of the amine (example 58) in 2 mL of Methanol was added 1 mL of H₂O, K₂CO₃ (11.4 mg) and 1-ethyl-1-methyl-4-oxopiperidinium iodide (17 mg). The resulting solution was heated at reflux for 1 h. The solution was poured into water and the aqueous phase was extracted with CH₂Cl₂. The crude was purified on prep TLC plate with MeOH/CH₂Cl₂ (5/95) to give the title compound. MS: 569 (M+1).

### EXAMPLE 67

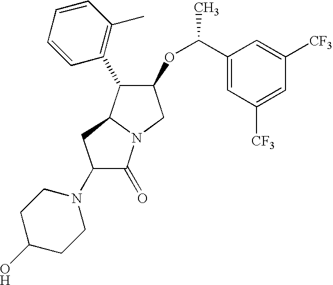

### (6R,7S,7aS)-6-{(1R)-1-[3,5-bis(trifluoromethyl)phenyl]ethoxy}-2-(4-hydroxypiperidin-1-yl)-7-(2-methylphenyl)hexahydro-3H-pyrrolizin-3-one

A solution of 12.8 mg of (6R,7S,7aS)-6-{(1R)-1-[3,5-bis(trifluoromethyl)phenyl]ethoxy}-7-(2-methylphenyl)-2-(4-oxopiperidin-1-yl)hexahydro-3H-pyrrolizin-3-one in 2 mL of methanol was added NaBH₄ and the solution was stirred at rt for 0.5 b. Upon removal of volatiles, the residue was purified by reverse phase HPLC to afford the title compound MS: 571.

### EXAMPLE 68

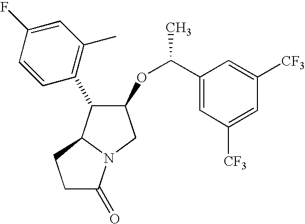

### (6R,7S,7aS)-6-{(1R)-1-[3,5-bis(trifluoromethyl)phenyl]ethoxy}-7-(4-fluoro-2-methylphenyl)hexahydro-3H-pyrrolizin-3-one

The title compounds were prepared as in example 2. MS: 490 (M+1).

### EXAMPLE 69 and EXAMPLE 70

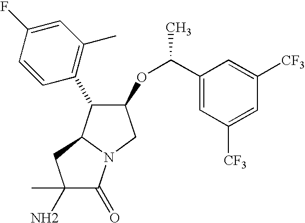

### (6R,7S,7aS)-2 (R or S)-amino-6-{(1R)-1-[3,5-bis(trifluoromethyl)phenyl]ethoxy}-7-(4-fluoro-2-methylphenyl)-2-methylhexahydro-3H-pyrrolizin-3-one

The title compounds were prepared as in examples 27 and 28. MS: 519 (M+1).

### EXAMPLE 71 and EXAMPLE 72

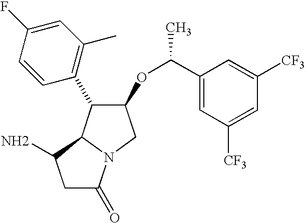

### (6R,7S,7aR)-(1R or S)-amino-6-{(1R)-1-[3,5-bis(trifluoromethyl)phenyl]ethoxy}-7-(4-fluoro-2-methylphenyl)hexahydro-3H-pyrrolizin-3-one

The title compounds were prepared as in examples 45 and 46 The two isomers were labeled D1 (less polar on TLC with MeOH/CH₂Cl₂=5/95) and D2 (more polar). MS: 505 (M+1).

### EXAMPLE 73

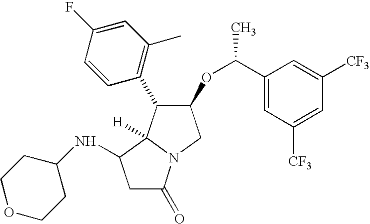

### (6R,7S,7aR)-6-{(1R)-1-[3,5-bis(trifluoromethyl)phenyl]ethoxy}-7-(4-fluoro-2-methylphenyl)-1-(tetrahydro-2H-pyran-4-ylamino)hexahydro-3H-pyrrolizin-3-one

The title compound was prepared from D2 (example 73) by reductive amination reaction. MS: 589 (M+1).

### EXAMPLE 74

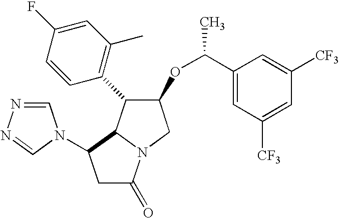

### (6R,7S,7aR)-6-{1 (1R)-1-[3,5-bis(trifluoromethyl)phenyl]ethoxy}-7-(4-fluoro-2-methylphenyl)-1-(4H-1,2,4-triazol-4-yl)hexahydro-3H-pyrrolizin-3-one

The title compound was prepared from D2 (example 73) by the method described in example 22. MS: 557 (M+1).

### EXAMPLE 75

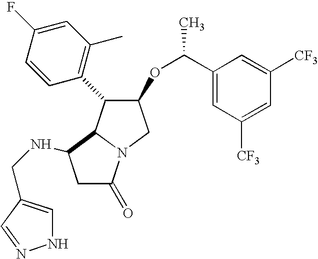

### (6R,7S,7aR)-6-{(1R)-1-[3,5-bis(trifluoromethyl)phenyl]ethoxy}-7-(4-fluoro-2-methylphenyl)-1-[(1H-pyrazol-4-ylmethyl)amino]hexahydro-3H-pyrrolizin-3-one

The title compound was prepared from D2 (example 73) by reductive amination. MS: 585 (M+1).

### EXAMPLE 76

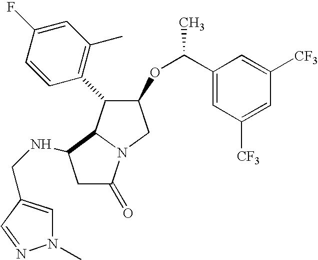

### (6R,7S,7aR)-6-{(1R)-1-[3,5-bis(trifluoromethyl)phenyl]ethoxy}-7-(4-fluoro-2-methylphenyl)-1-{[(1-methyl-1H-pyrazol-4-yl)methyl]amino}hexahydro-3H-pyrrolizin-3-one

The title compound was prepared from D2 (example 73) by reductive amination. MS: 599 (M+1).

### EXAMPLE 77

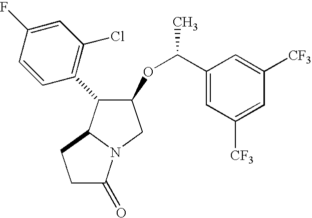

### (6R,7S,7aS)-6-{(1R)-1-[3,5-bis(trifluoromethyl)phenyl]ethoxy}-7-(2-chloro-4-fluorophenyl)hexahydro-3H-pyrrolizin-3-one

The title compounds were prepared as in example 2. MS: 510 (M+1).

### EXAMPLE 78 and EXAMPLE 79

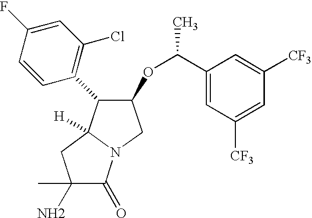

### (6R,7S,7aS)-2 (R or S)-amino-6-{(1R)-1-[3,5-bis(trifluoromethyl)phenyl]ethoxy}-7-(2-chloro-4-fluorophenyl)-2-methylhexahydro-3H-pyrrolizin-3-one

The title compounds were prepared as in examples 27 and 28. MS: 539 (M+1).

### EXAMPLE 80 and EXAMPLE 81

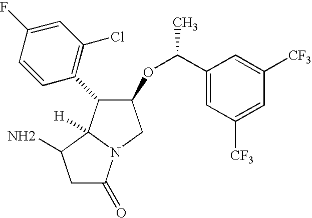

### (6R,7S,7aR)-1(R or S)-amino-6-{(1R)-1-[3,5-bis(trifluoromethyl)phenyl]ethoxy}-7-(2-chloro-4-fluorophenyl)hexahydro-3H-pyrrolizin-3-one

The title compounds were prepared as in examples 45 and 46. The two isomers were labeled D1 (less polar on TLC with MeOH(CH₂Cl₂=5/95) and D2 (more polar). MS: 525 (M+1).

### EXAMPLE 82

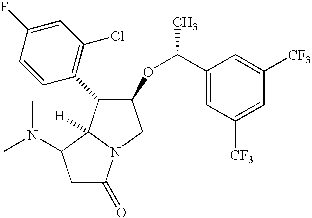

### (6R,7S,7aR)-6-{(1R)-1-[3,5-bis(trifluoromethyl)phenyl]ethoxy}-7-(2-chloro-4-fluorophenyl)-1-(dimethylamino)hexahydro-3H-pyrrolizin-3-one

The title compounds were prepared as in example 8 from D1 isomer in example 80. MS: 553 (M+1).

### EXAMPLE 83

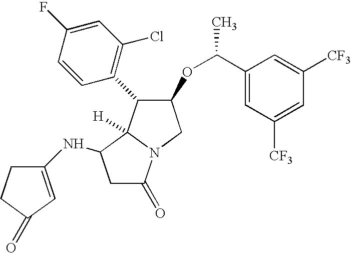

### (6R,7S,7aR)-6-{(1R)-1-[3,5-bis(trifluoromethyl)phenyl]ethoxy}-7-(2-chloro-4-fluorophenyl)-1-[(3-oxocyclopent-1-en-1-yl)amino]hexahydro-3H-pyrrolizin-3-one

A solution of amine (5 mg, D1 isomer in example 80), TsOH (4 mg) and cyclopentane-1,3-dione (5 mg) in 2 mL of toluene was heated at reflux for 3 h. Upon removal of volatiles, the residue was purified by reverse phase HPLC. MS: 605 (M+1).

### EXAMPLE 84

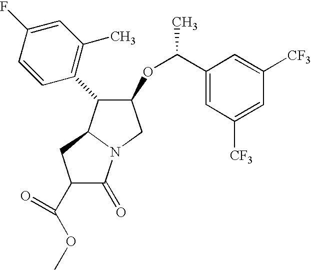

### methyl (6R,7S,7aS)-6-{(1R)-1-[3,5-bis(trifluoromethyl)phenyl]ethoxy}-7-(4-fluoro-2-methylphenyl)-3-oxohexahydro-1H-pyrrolizine-2-carboxylate

### Step A: methyl (6R,7S,7aS)-6-{(1R)-1-[3,5-bis(trifluoromethyl)phenyl]ethoxy}-7-(4-fluoro-2-methylphenyl)-3-oxohexahydro-1H-pyrrolizine-2-carboxylate

The title compound was prepared as a 1:1 mixture of diastereomers according to procedure for Example 23. ¹H NMR: 7.71 (s, 1H), 7.44 (s, 1H), 7.42 (s, 1H), 6.82-6.97 (m, 3H), 4.43 (q, 1H, J=6.4 Hz), 4.06-4.16 (m, 1.5H), 3.90 (q, 0.5H, J=8.5 Hz), 3.83 and 3.77 (s, 3H), 3.74-3.81 (m, 1H), 3.53-3.61 (m, 2H), 3.38 (t, 0.5H, J=8.1 Hz), 3.21 (t, 0.5H, J=8.0 Hz), 2.55-2.59 (m, 0.5H), 2.38-2.43 (m, 1H), 2.37 and 2.33 (s, 3H), 2.06-2.12 (m, 0.5H), 1.38-1.40 (m, 3H).

### EXAMPLE 85 and 86

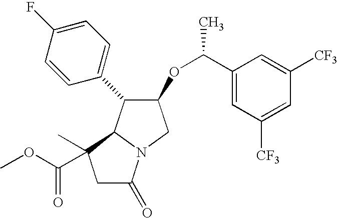

### methyl (6R,7S,7aR)-6-{(1R)-1-[3,5-bis(trifluoromethyl)phenyl]ethoxy}-7-(4-fluorophenyl)-1-methyl-3-oxohexahydro-1H-pyrrolizine-1-carboxylate

### Step A: tert-butyl (2R,3S,4R)-4-{(1R)-1-[3,5-bis(trifluoromethyl)phenyl]ethoxy}-3-(4-fluorophenyl)-2-formylpyrrolidine-1-carboxylate

To a stirred solution of 7.26 mL (14.51 mmol) oxalyl chloride in 140 mL dry methylene chloride under nitrogen atmosphere at −78° C. was added 1.93 mL (29.02 mmol) DMSO dropwise over 15 min by syringe. After ten min., to this mixture was added a solution of 4 g (7.26 mmol) tert-butyl 4-{1-[3,5-bis(trifluoromethyl)phenyl]ethoxy}-2-(hydroxymethyl)-3-phenylpyrrolidine-1-carboxylate (Example 1) in 5 mL dry methylene chloride. The reaction mixture was stirred at −78° C. for 1 hr, then 8.13 mL (58.04 mmol) TEA was added by syringe. The reaction mixture was stirred at −78° C. for 15 min then warmed to room temperature and stirred an additional hr. The reaction mixture was quenched with aq. 1N HCL (˜100 mL) and transferred to a separatory funnel. The reaction mixture was extracted with EtOAc (2×100 mL). The combined organic extracts were washed with water (100 mL) then brine (100 mL), dried over anhydrous sodium sulfate, filtered and evaporated under vacuum afford the title compound. The resulting crude product was purified by Horizon MPLC purification using a stepwise gradient eluant of ethyl acetate in hexane (0-50%) to afford 3.74 g (94%) of the title compound.

### Step B (2R,3S,4R)-4-{(1R)-1-[3,5-bis(trifluoromethyl)phenyl]ethoxy}-1-(tert-butoxycarbonyl)-3-(4-fluorophenyl)pyrrolidine-2-carboxylic acid

To a solution of 2.00 mg (3.64 mmol) tert-butyl (2R,3S,4R)-4-{(1R)-1-[3,5-bis(trifluoromethyl)phenyl]ethoxy}-3-(4-fluorophenyl)-2-formylpyrrolidine-1-carboxylate (intermediate from step A) in 125 mL of tert-butanol/2-methylbut-2-ene (4/1) was added NaH₂PO₄ (3.26 g, 23.67 mmol) and NaClO₂ (2.83 g, 31.30 mmol) in 40 mL water and the resulting solution was stirred for one hour at room temperature. The reaction was diluted with dichloromethane (200 mL), separated, and the aqueous then extracted with dichloromethane (2×200 mL). The organics were combined and treated as stated in Example 3, step E. The residue was purified by silica gel flash column eluting with methanol/dichloromethane (1/9) to afford 2.02 g (97%) of the title compound. MS: 566 (M+H) and 466 (M-Boc)

### Step C tert-butyl (2R,3S,4R)-4-{(1R)-1-[3,5-bis(trifluoromethyl)phenyl]ethoxy}-2-(diazenylacetyl)-3-(4-fluorophenyl)-2-formylpyrrolidine-1-carboxylate

To a solution of (2R,3S,4R)-4-{(1R)-1-[3,5-bis(trifluoromethyl)phenyl]ethoxy}-1-(tert-butoxycarbonyl)-3-(4-fluorophenyl)pyrrolidine-2-carboxylic acid (2.00 g, 3.53 mmol) in THF (20 mL) at 0° C. under nitrogen atmosphere was added via syringe iso-butylchloroformate (0.70 mL, 5.30 mmol) and TEA (0.840 mL, 6.00 mmol) and the solution turned milky. After stirring for 1 h at 0° C., pre-made diazomethane (˜68 mmol in 50 mL ether) was distilled into the reaction and the mixture stirred for 1 h at 0° C. The mixture was then allowed to warm to room temperature and stirred for an additional hour. The solution was placed in an ice/water bath and the solution quenched with acetic acid. The mixture was poured into ether (100 mL), washed with saturated sodium bicarbonate, brine; dried over sodium sulfate, filtered and concentrated. Toluene was used to azeotrop the remaining water. The residue was purified by Horizon MPLC using a stepwise gradient eluant of ethyl acetate in hexane (0-50%) to afford 1.38 g (70%) of the title compound.

### Step D tert-butyl (2R,3S,4R)-4-{(1R)-1-[3,5-bis(trifluoromethyl)phenyl]ethoxy}-3-(4-fluorophenyl)-2-(2-methoxy-2-oxoethyl)pyrrolidine-1-carboxylate

To a solution of tert-butyl (2R,3S,4R)-4-{(R)-1-[3,5-bis(trifluoromethyl)phenyl]ethoxy}-2-(diazenylacetyl)-3-(4-fluorophenyl)-2-formylpyrrolidine-1-carboxylate (100 g, 1.70 mmol) in methanol (10 mL) was added via syringe TEA (0.711 mL, 5.08 mmol) followed by solid silver benzoate (25 mg, 0.08 mmol) and the resulting solution set under nitrogen atmosphere and stirred at room temperature overnight. The mixture was evaporated and the residue purified by Horizon MPLC using a stepwise gradient eluant of ethyl acetate in hexane (0-70%) to afford 835 mg (83%) of the title compound. MS: 594 (M+H)

### Step E tert-butyl (2R,3S,4R)-4-{(1R)-1-[3,5-bis(trifluoromethyl)phenyl]ethoxy}-3-(4-fluorophenyl)-2-(2-methoxy-1-methyl-2-oxoethyl)pyrrolidine-1-carboxylate

To a solution of tert-butyl (2R,3S,4R)-4-{(1R)-1-[3,5-bis(trifluoromethyl)phenyl]ethoxy}-3-(4-fluorophenyl)-2-(2-methoxy-2-oxoethyl)pyrrolidine-1-carboxylate (600 mg, 1.00 mmol) in dry THF (10 mL) cooled to −78° C. via dry ice/acetone bath under nitrogen atmosphere was added dropwise via syringe a 1.0M solution of lithium bis(trimethylsilyl)-amide in THF (1.50 mL, 1.50 mmol) and the resulting solution stirred for 1 h at −78° C. Methyl iodide (0.190 mL, 3.00 mL) was introduced via syringe and the resulting mixture stirred for 30 minutes allowing to warm to −40° C. and stirred for an additional 2 hours. Reaction was quenched with saturated ammonium chloride solution and extracted with ethyl acetate (2×20 mL). Combine the organics, dry over magnesium sulfate, filter and concentrate. The residue was purified by preparative TLC plate eluting with acetone/hexane (1/8) to afford 400 mg (66%) of the title compound. MS: 608 (M+H).

### Step F tert-butyl (2R,3S,4R)-4-{(1R)-1-[3,5-bis(trifluoromethyl)phenyl]ethoxy}-3-(4-fluorophenyl)-2-[1-(methoxycarbonyl)-1-methylbut-3-en-1-yl]pyrrolidine-1-carboxylate

The title compound was prepared as in Example 85 and 86, Step E; however, replacing the methyl iodide with allyl bromide as the alkylating reagent. MS: 648 (M+H).

### Step G tert-butyl (2R,3S,4R)-4-{(1R)-1-[3,5-bis(trifluoromethyl)phenyl]ethoxy}-3-(4-fluorophenyl)-2-[1-(methoxycarbonyl)-1-methyl-3-oxopropyl]pyrrolidine-1-carboxylate

The title compound was similarly prepared as in Example 85 and 86, Step A to afford 117 mg (65%) as a yellow oil. MS: 650 (M+H).

### Step H 3-[(2R,3S,4R)-4-{(1R)-1-[3,5-bis(trifluoromethyl)phenyl]ethoxy}-1-(tert-butoxycarbonyl)-3-(4-fluorophenyl)pyrrolidin-2-yl)-4-methoxy-3-methyl-4-oxobutanoic acid

The title compound was similarly prepared as in Example 85 and 86, Step B to afford carboxylic acid 100 mg (87%) as a clear film. MS: 666 (M+H) and 648 (M-OH).

### Step I 3-[(2R,3S,4R)-4-{(1R)-1-[3,5-bis(trifluoromethyl)phenyl]ethoxy}-3-[(4-fluorophenyl)pyrrolidin-2-yl]-4-methoxy-3-methyl-4-oxobutanoic acid hydrochloride

3-[(2R,3S,4R)-4-{(1R)-1-[3,5-bis(trifluoromethyl)phenyl]ethoxy}-1-(tert-butoxycarbonyl)-3-(4-fluorophenyl)pyrrolidin-2-yl)-4-methoxy-3-methyl-4-oxobutanoic acid (100 mg, 0.150 mmol) is dissolved in a solution of 4N hydrochloric acid in dioxane (5 mL) with 0.5 mL water added and the resulting solution is stirred at room temperature for 2 hours. Concentrate to dryness and azeotrop with toluene (3×5 mL) to remove all water. No further purification was done on the compound. The compound was dried overnight to give a white powder (90 mg, quantitative). MS: 566 (M+H).

### Step J methyl (6R,7S,7aR)-6-{(1R)-1-[3,5-bis(trifluoromethyl)phenyl]ethoxy}-7-(4-fluorophenyl)-1-methyl-3-oxohexahydro-1H-pyrrolizine-1-carboxylate

To a solution of 3-[(2R,3S,4R)-4-{(1R)-1-[3,5-bis(trifluoromethyl)phenyl]ethoxy}-3-[(4-fluorophenyl)pyrrolidin-2-yl]-4-methoxy-3-methyl-4-oxobutanoic acid hydrochloride (90 mg, 0.150 mmol) in dichloromethane (2 mL) was added DIEA (0.026 mL, 0.15 mmol), DMAP (4 mg, 0.03 mmol) and EDC (58 mg, 0.30 mmol) and the resulting solution stirred overnight at room temperature under nitrogen atmosphere. The organics were combined and treated as stated in Example 3, step E. The residue was purified by preparative TLC plate eluting with ethyl acetate/hexane (1/4) to afford two separate diasteromers D1 (example 85, 51 mg, 60%) and D2 (example 86, 11 mg, 13%). MS: of both compounds 548 (M+H).

### EXAMPLE 87 and 88

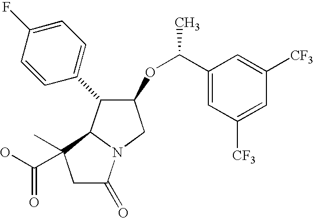

### (6R,7S,7aR)-6-{(1R)-1-[3,5-bis(trifluoromethyl)phenyl]ethoxy}-7-(4-fluorophenyl)-1-methyl-3-oxohexahydro-1H-pyrrolizine-1-carboxylic acid

Step A: To a solution of D1 (methyl (6R,7S,7aR)-6-{(1R)-1-[3,5-bis(trifluoromethyl)phenyl]ethoxy}-7-(4-fluorophenyl)-1-methyl-3-oxohexahydro-1H-pyrrolizine-1-carboxylate) from example 85 and 86 (50 mg, 0.09 mmol) in methanol, tetrahydrofuran, and water (1:1:1 solution, 4.5 mL) was added 11 mg (0.45 mmol) and the resulting solution heated to 60° C. overnight. Cool to room temperature and concentrate in vacuo. Take the aqueous layer and add 1N HCl dropwise until pH of 4 is reached. Extract with ethyl acetate (4×5 mL), combine organics, dry over sodium sulfate, filter and concentrate. Purification by Gilson prep-reverse phase HPLC afforded 40 mg (80%) of diastereomer 1 (D1). MS: 534 (M+H).
Step B: Diastereomer 2 (D2) was similarly prepared as in Example 87 and 88, Step A to afford the carboxylic acid 8.2 mg (82%) as a clear film. MS: 534 (M+H).

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

What is claimed is:

1. A compound of the formula I, II or III: 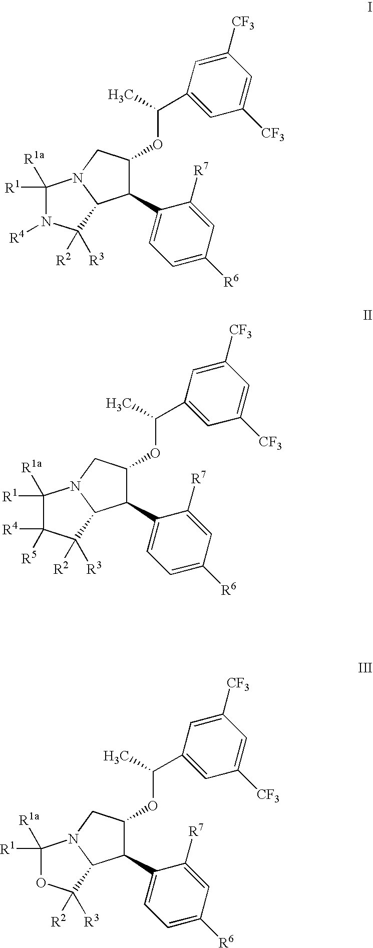
wherein:
R¹ and R^{1a} are each hydrogen or together with the carbon atom to which they are attached form a carbonyl;
Each R² is selected from the group consisting of:
(1) hydrogen,
(2) NH₂, and
(3) CH₃;
R³ are each independently selected from the group consisting of:
(1) hydrogen,
(2) hydroxyl,
(3) NH₂,
(4) N(CH₃)₂;
(5) NH—C(O)—C(CH₃)₂—NH₂,
(6) NH—C(O)—CF₃,
(7) C₁₋₆alkyl,
(8) C₁₋₆alkyl-O—C₁₋₆alkyl, and
(9) -A1, wherein A1 is a heteroaryl or heterocycle of 5 or 6 atoms wherein the heteroaryl or heterocyle contains 1, 2 or 3 heteroatoms selected from the group consisting of O, N and S, and wherein the heteroaryl or heterocylce is optionally substituted with a group selected from methyl, oxo and hydroxyl,
(10) —NH-A1
(11) —NH—CH₂-A1,
(12) CO₂Me; and
(13) CO₂H;
R⁴ and R⁵ are each independently selected from a group consisting of:
(1) hydrogen,
(2) hydroxy,
(3) hydroxyC₁₋₃alkyl,
(4) C₁₋₃alkyl,
(5) —C(O)—O—CH₃,
(6) NH₂,
(7) aminoC₁₋₃alkyl,
(8) N(R⁹)(R¹⁰),
(9) A2, wherein A2 is selected from the group consisting of 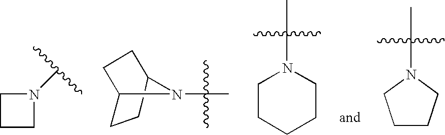
wherein A2 is optionally substituted with a group selected from hydroxyl, methyl, COOH, —COO—C₁₋₄alkyl and 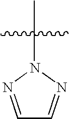
(10) A3, wherein A3 is a heteraromatic or heterocyclic ring of 5 or 6 atoms, wherein 1, 2, or 3 of the atoms is a heteroatom selected from N, S or O, and wherein at least one of the heteroatoms is a N, and wherein the heteroaryl or heterocycle is optionally substituted with a group selected from methyl, oxo, hydroxyl, —CH₃—NH₂ and —CH₃—N(CH₃)₂,
(11) C₁₋₃-A2,
(12) C₁₋₃-A3, and
or R⁴ and R⁵ together with the carbon to which they are attached form a carbonyl;
R⁶ and R⁷ are each independently selected from a group consisting of:
(1) hydrogen,
(2) halo, and
(3) methyl;
R⁹ and R¹⁰ are each selected from the group consisting of
(1) hydrogen,
(2) methyl,
(3) A4, wherein A4 is a heteroaryl or heterocycle of 5 or 6 atoms wherein the heteroaryl or heterocyle contains 1, 2 or 3 heteroatoms selected from the group consisting of O, N and S, and wherein the heteroaryl or heterocycle is optionally substituted with a group selected from methyl, oxo, hydroxyl, —CH₃—NH₂ and —CH₃—N(CH₃)₂, and
(4) —C₁₋₃alkyl-A4,
or a pharmaceutically acceptable salts thereof and individual enantiomers and diastereomers thereof.

2. A compound according to claim 1 of formula II

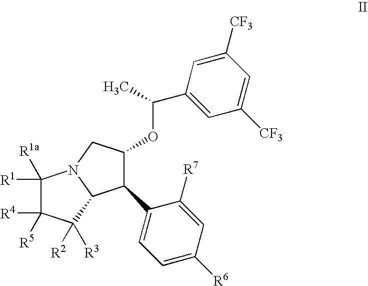

3. A compound according to claim 1 wherein
A1 is selected from the group consisting of

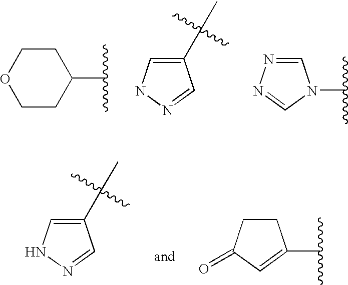

4. A compound according to claim 1 wherein
R³ is selected from the group consisting of
(1) NH₂, and
(2) —NH-A1, wherein A1 is selected from the group consisting of 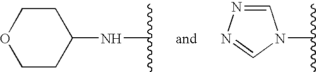
and A1 is optionally substituted with a group selected from methyl, oxo and hydroxyl.

5. A compound according to claim 1 wherein A3 and A4 are each selected from the group consisting of 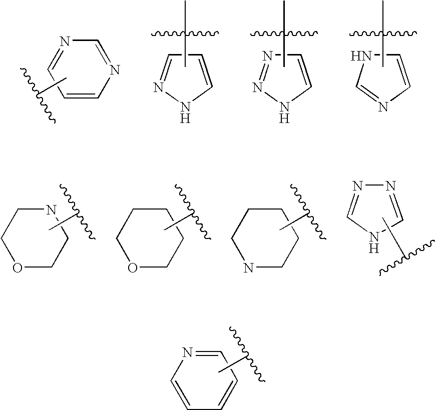
wherein A4 is optionally substituted with a substituent selected from the group consisting of hydroxyl, oxo, methyl, —CH₃—NH₂ and CH₃—N(CH₃)₂.

6. A compound according to claim 1 wherein
R⁴ and R⁵ are each independently selected from a group consisting of:
(1) hydroxy,
(2) NH₂, and
(3) N(R⁹)(R¹⁰).

7. A compound according to claim 1 wherein
R⁶ and R⁷ are each independently selected from a group consisting of:
(1) hydrogen,
(2) fluoro, and
(3) methyl.

8. A compound according to claim 1 wherein
R⁹ and R¹⁰ are each selected from the group consisting of
(1) hydrogen, and
(2) -A4, wherein A4 is selected from the group consisting of 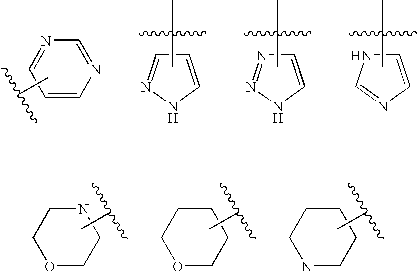
wherein A4 is optionally substituted with a substituent selected from the group consisting of hydroxyl, oxo, methyl, —CH₃—NH₂ and —CH₃—N(CH₃)₂.

9. A compound according to claim 1 wherein
Each R² is selected from the group consisting of:
(1) hydrogen, and
(2) CH₃.

10. A compound according to claim 1 wherein
R⁶ is fluoro and R⁷ is methyl.

11. A compound according to claim 2 wherein
R¹ and R^{1a} are each hydrogen or together with the carbon atom to which they are attached form a carbonyl;
each R² is selected from the group consisting of:
(1) hydrogen, and
(2) CH₃;
R³ is selected from the group consisting of
(1) NH₂, and
(2) —NH-A1, wherein A1 is selected from the group consisting of 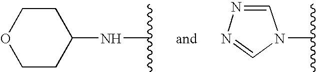
and A1 is optionally substituted with a group selected from methyl, oxo and hydroxyl;
R3 is selected from the group consisting of
(1) NH₂, and
(2) —NH-A1, wherein A1 is selected from the group consisting of 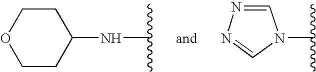
and A1 is optionally substituted with a group selected from methyl, oxo and hydroxyl;
R⁴ and R⁵ are each independently selected from a group consisting of:
(1) hydroxy,
(2) NH₂, and
(3) N(R⁹)(R¹⁰).
R⁶ and R⁷ are each independently selected from a group consisting of:
(1) hydrogen,
(2) fluoro, and
(3) methyl;
R⁹ and R¹⁰ are each selected from the group consisting of
(1) hydrogen,
(2) methyl, and
(3) A4, wherein A1 is a heteroaryl or heterocycle of 5 or 6 atoms wherein the heteroaryl or heterocyle contains 1, 2 or 3 heteroatoms selected from the group consisting of O, N and S, and wherein the heteroaryl or heterocylce is optionally substituted with a group selected from methyl, oxo, hydroxyl, —CH₃—NH₂ and —CH₃—N(CH₃)₂,
(4) —C₁₋₃alkyl-A4,
or a pharmaceutically acceptable salts thereof and individual enantiomers and diastereomers thereof.

12. A compound according to claim 1 of formula I or III

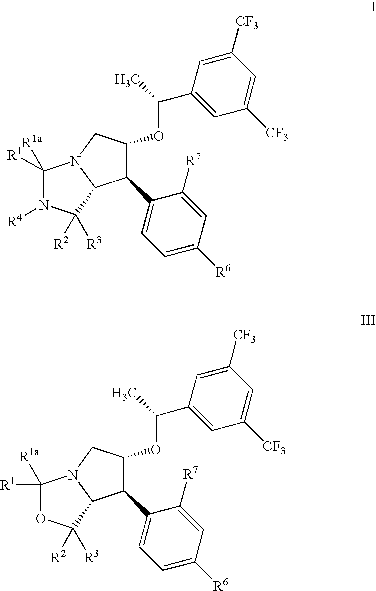

13. A compound according to claim 12 wherein
R¹ and R^{1a} are each hydrogen or together with the carbon atom to which they are attached form a carbonyl;
Each R² is selected from the group consisting of:
(1) hydrogen, and
(2) CH₃;
R³ are each independently selected from the group consisting of:
(1) hydrogen,
(2) hydroxyl,
(3) C₁₋₆alkyl, and
(4) C₁₋₆alkyl-O—C₁₋₆alkyl;
R⁴ and R⁵ are each independently selected from a group consisting of:
(1) hydroxy,
(2) NH₂, and
(3) N(R⁹)(R¹⁰).
R⁶ and R⁷ are each independently selected from a group consisting of:
(1) hydrogen,
(2) fluoro, and
(3) methyl;
R⁹ and R¹⁰ are each selected from the group consisting of
(1) hydrogen,
(2) methyl, and
(3) A4, wherein A1 is a heteroaryl or heterocycle of 5 or 6 atoms wherein the heteroaryl or heterocyle contains 1, 2 or 3 heteroatoms selected from the group consisting of O, N and S, and wherein the heteroaryl or heterocylce is optionally substituted with a group selected from methyl, oxo, hydroxyl, —CH₃—NH₂ and CH₃—N(CH₃)₂,
(4) —C₁₋₃alkyl-A4,
or a pharmaceutically acceptable salts thereof and individual enantiomers and diastereomers thereof.

14. A compound which is selected from the group consisting of: 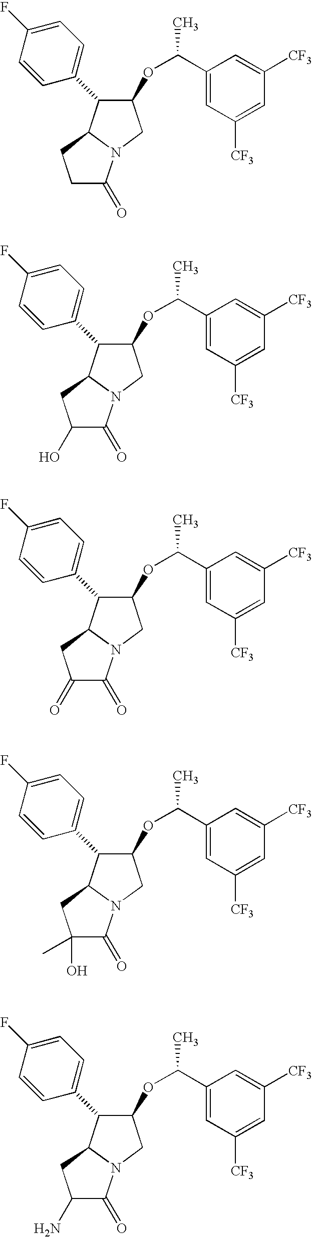 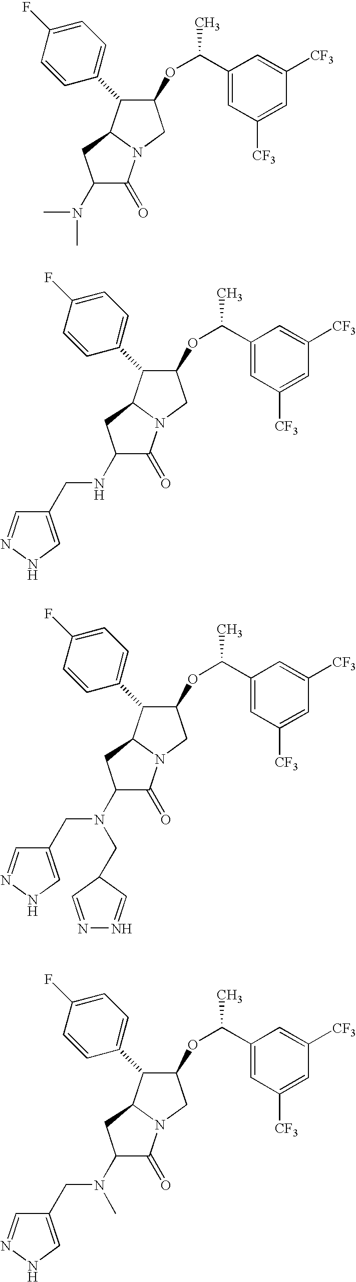 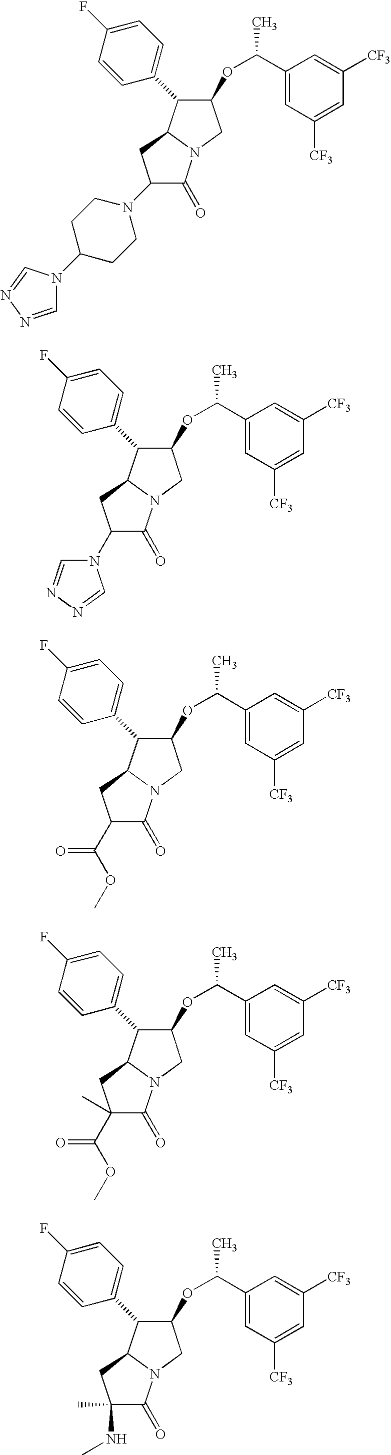 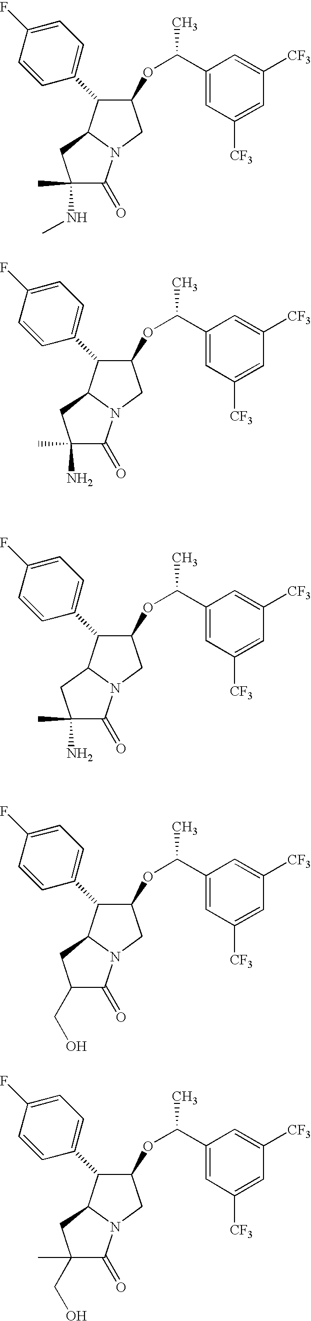 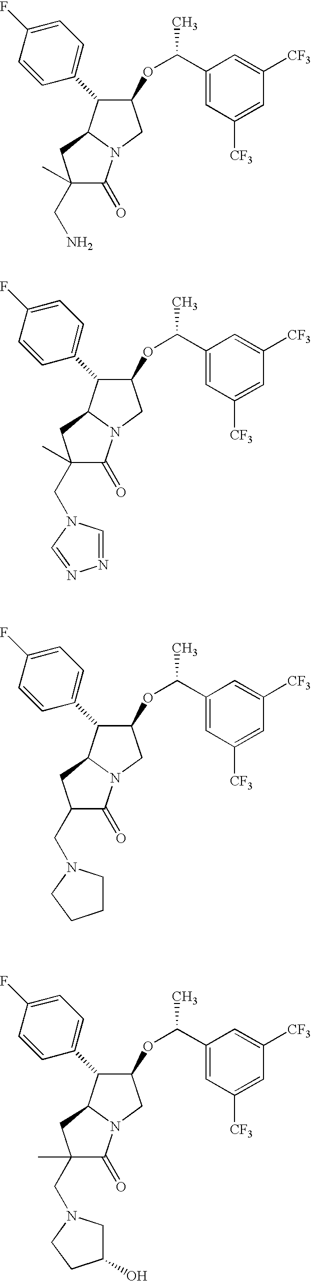 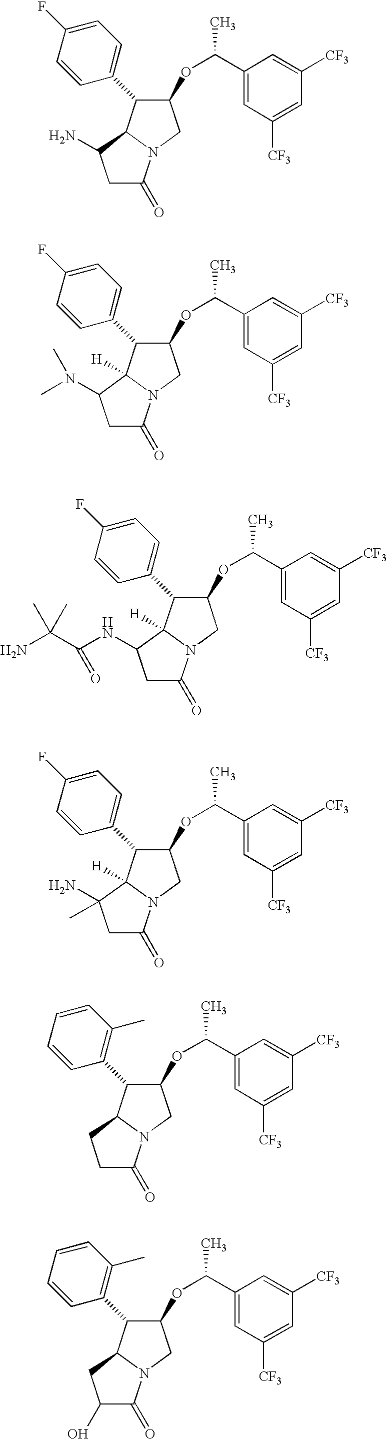 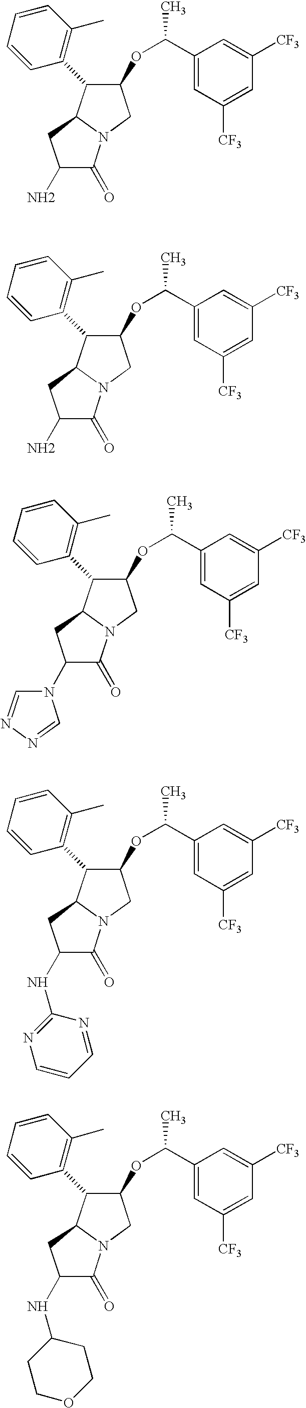 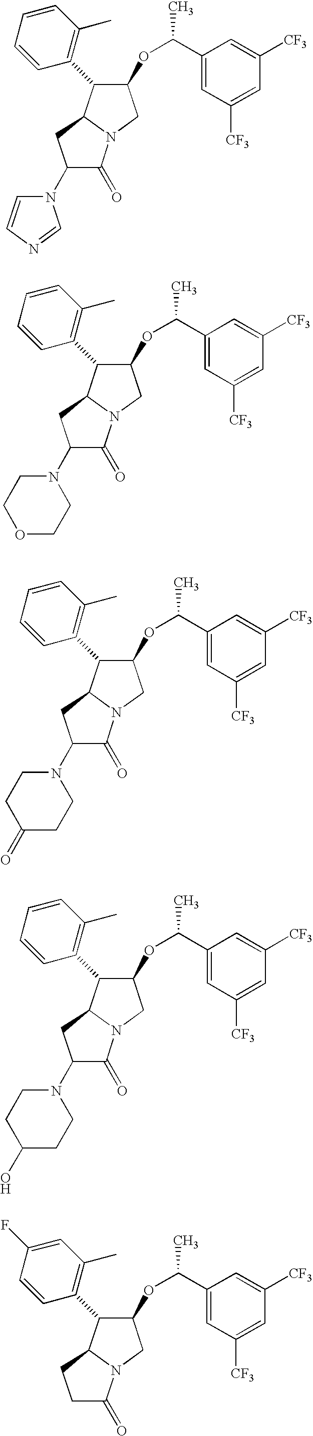 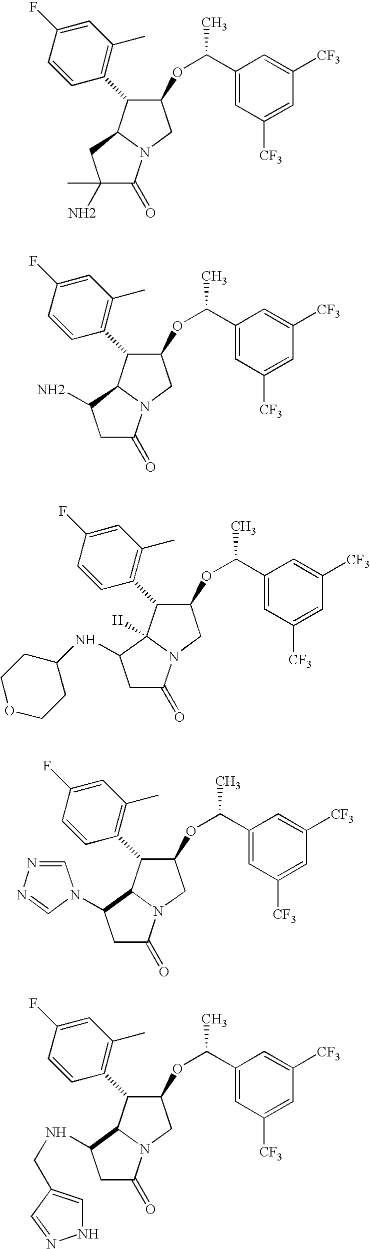 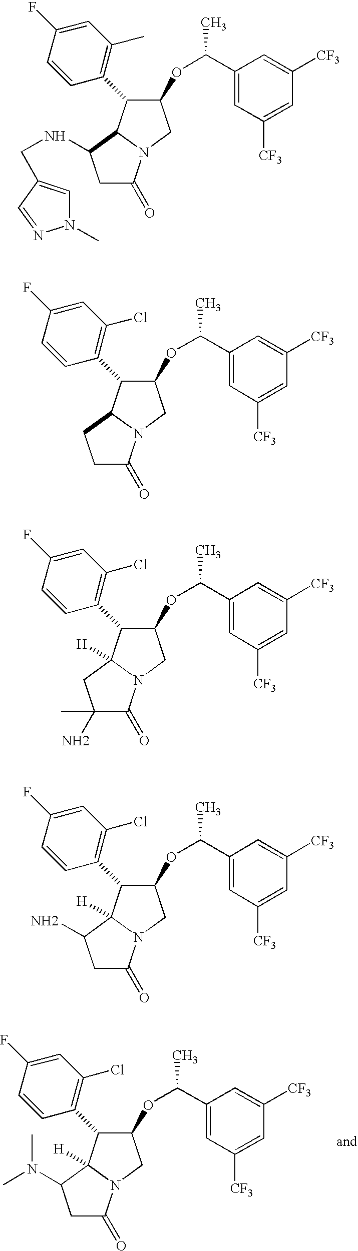 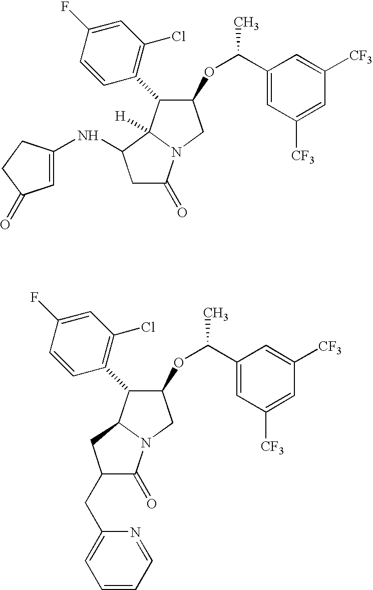
or a pharmaceutically acceptable salt thereof.

15. A compound according to claim 1 of the formula 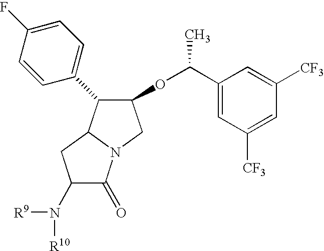
wherein
R⁹ and R¹⁰ are 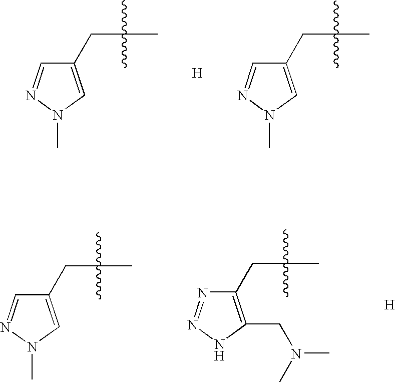
or a pharmaceutically acceptable salt thereof.

16. A compound according to claim 1 of the formula 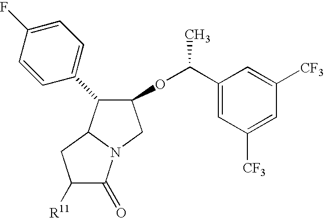
wherein
R¹¹ is 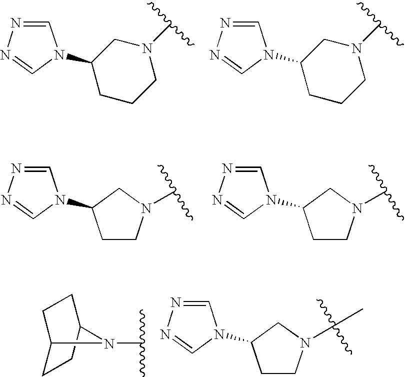
or a pharmaceutically acceptable salt thereof.

17. A compound according to claim 1 of the formula 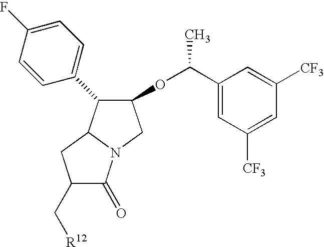
wherein
R¹² is 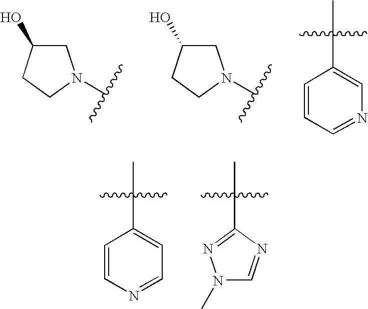
or a pharmaceutically acceptable salt thereof.

18. A compound according to claim 1 of formula 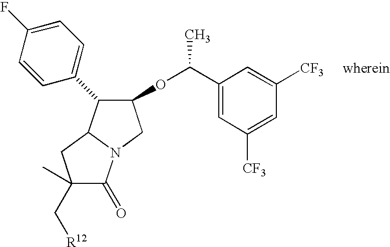
R¹² is 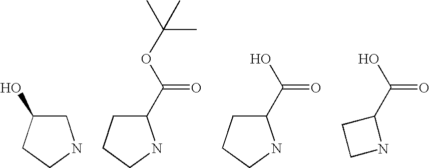
or a pharmaceutically acceptable salt thereof.

19. A compound according to claim 1 of formula 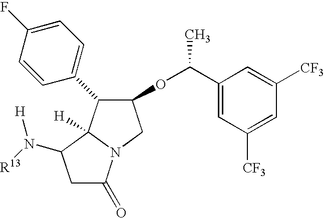
wherein
R¹³ is 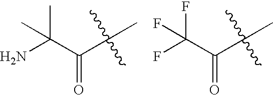
or a pharmaceutically acceptable salt theory

20. A pharmaceutical composition which comprises an inert carrier and a compound of claim 1 or a pharmaceutically acceptable salt thereof.

21. A method for the manufacture of a medicament for antagonizing the effect of substance P at its receptor site or for the blockade of neurokinin-1 receptors in a mammal comprising combining a compound of claim 1 or a pharmaceutically acceptable salt thereof with a pharmaceutical carrier or diluent.

22. A method for the manufacture of a medicament for the treatment of a physiological disorder associated with an excess of tachykinins in a mammal comprising combining a compound of claim 1 or a pharmaceutically acceptable salt thereof with a pharmaceutical carrier or diluent.
